(19) United States
(12) Patent Application Publication
(10) Pub. No.: US 2023/0383291 A1
(43) Pub. Date: Nov. 30, 2023

(54) PEPTIDE-BASED TRANSDUCTION OF NON-ANIONIC POLYNUCLEOTIDE ANALOGS FOR GENE EXPRESSION MODULATION

(71) Applicant: FELDAN BIO INC., Québec (CA)

(72) Inventors: Jean-Pascal LEPETIT-STOFFAES, Québec (CA); Nancy MESSIER, Québec (CA); David GUAY, Québec (CA); Thomas DEL'GUIDICE, Québec (CA); Xavier BARBEAU, Québec (CA); Stéphanie HALLÉE, Ste-Catherine-de-la-Jacques-Cartier (CA)

(21) Appl. No.: 18/032,329

(22) PCT Filed: Oct. 18, 2021
(86) PCT No.: PCT/CA2021/051458

§ 371 Date: Apr. 17, 2023

Related U.S. Application Data
(60) Provisional application No. 63/104,263, filed on Oct. 22, 2020.
(60) Provisional application No. 63/093,295, filed on Oct. 18, 2020.

(51) Int. Cl. C12N 15/113; C12N 15/87

(52) U.S. Cl. CPC C12N 15/113; C12N 15/87; C12N 2310/11; C12N 2310/314; C12N 2310/3233; C12N 2310/3513; C12N 2320/32; C12N 2810/40

(57) ABSTRACT

Compositions and methods for delivering non-anionic polynucleotide analog cargoes to the cytosolic/nuclear compartment of eukaryotic cells via a synthetic peptide shuttle agent are described herein. The non-anionic polynucleotide analog cargoes may be charge-neutral or cationic, and the synthetic peptide shuttle agent is a peptide comprising an amphipathic alpha-helical motif having both a positively-charged hydrophilic outer face and a hydrophobic outer face, wherein synthetic peptide shuttle agent is not covalently linked, or linked in a cleavable manner under physiological conditions, to the non-anionic polynucleotide analog cargoes. The non-anionic polynucleotide analog cargo may be a charge-neutral or cationic antisense synthetic oligonucleotide (ASO) that hybridizes to an intracellular target RNA for gene expression modification.

### CROSS-REFERENCE TO RELATED APPLICATIONS

This application is a U.S. National Stage Application of PCT Application No. PCT/CA2021/051458, filed Oct. 18, 2021, which claims the benefit of U.S. Application No. 63/104,263, filed Oct. 22, 2020, and U.S. Application No. 63/093,295, filed Oct. 18, 2020, all of which are incorporated herein by reference in their entirety.

The present description relates to the intracellular delivery of non-anionic polynucleotide analog cargoes. More specifically, the present description relates to the use of synthetic peptide shuttle agents for the intracellular delivery of non-anionic polynucleotide analog cargoes.

The present description refers to a number of documents, the contents of which are herein incorporated by reference in their entirety.

### SEQUENCE LISTING

The instant application contains a Sequence Listing which has been submitted electronically in .txt format and is hereby incorporated by reference in its entirety. Said .txt copy, created on Apr. 11, 2023, is named 49446_706_831_SL_txt and is 130,751 bytes in size.

### BACKGROUND

Most non-anionic polynucleotide analog cargoes suffer from issues relating to their intracellular delivery, often requiring their covalent conjugation to delivery moieties thereby making their synthesis and commercialization more complex. Improved methods of increasing the cytosolic/nuclear delivery of non-anionic polynucleotide analog cargoes are highly desirable.

### SUMMARY

Synthetic peptide shuttle agents represent a recently defined family of peptides previously reported to transduce proteinaceous cargoes quickly and efficiently to the cytosol and/or nucleus of a wide variety of target eukaryotic cells. In contrast to traditional cell penetrating peptide-based intracellular delivery strategies, synthetic peptide shuttle agents are not covalently linked to their polypeptide cargoes. In fact, covalently linking shuttle agents to their cargoes generally has a negative effect on their transduction activity. The first generation of such peptide shuttle agents was described in WO/2016/161516, wherein the peptide shuttle agents comprise an endosome leakage domain (ELD) operably linked to a cell penetrating domain (CPD). WO/2018/068135 subsequently described further synthetic peptide shuttle agents rationally-designed based on a set of fifteen design parameters for the sole purpose of improving the transduction of proteinaceous cargoes, while reducing toxicity of the first generation peptide shuttle agents.

While cell penetrating peptides (CPPs) have been used for decades in transfection strategies to deliver DNA/RNA intracellularly, first generation synthetic peptide shuttle agents, which contain a CPD derived from CPPs, were not able to efficiently transduce plasmid DNA cargo to the nucleus for gene expression, with the DNA cargoes largely remaining trapped in endosomes. Subsequent experiments revealed that second generation synthetic peptide shuttle agents were also not suitable for efficiently transducing plasmid DNA to the nucleus for gene expression. Furthermore, strategies involving neutralizing the negatively-charged phosphate backbone of DNA/RNA by coating with small positively charged molecules failed to significantly improve shuttle agent-mediated cargo transduction, with endosomal entrapment continuing to be problematic. This suggested that more than mere charge neutralization was required for shuttle agent-medicated transduction of polynucleotides.

The present disclosure relates to the surprising discovery that synthetic peptide shuttle agents have the ability to transduce non-anionic polynucleotide analog cargoes quickly and efficiently to the cytosolic/nuclear compartment in sufficient quantities for effecting gene expression modification in eukaryotic cells.

In one aspect, described herein is a composition comprising a non-anionic polynucleotide analog cargo for intracellular delivery and a synthetic peptide shuttle agent that is independent from, or is not covalently linked to, said non-anionic polynucleotide analog cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said non-anionic polynucleotide analog cargo in eukaryotic cells as compared to in the absence of the synthetic peptide shuttle agent.

In a further aspect, described herein is a method for modifying gene expression in eukaryotic cells, the method comprising: (a) providing a non-anionic polynucleotide analog cargo for intracellular delivery, the non-anionic polynucleotide analog cargo being designed to hybridize to an RNA of interest in the eukaryotic cells; (b) providing a synthetic peptide shuttle agent that is independent from, or is not covalently linked to, said non-anionic polynucleotide analog cargo; (c) contacting the eukaryotic cells with the non-anionic polynucleotide analog cargo in the presence of the synthetic peptide shuttle agent at a concentration sufficient to increase the transduction efficiency and/or cytosolic/nuclear delivery of the charge-neutral polynucleotide analog cargo, as compared to in the absence of said synthetic peptide shuttle agent, wherein the non-anionic polynucleotide analog cargo hybridizes to the RNA of interest upon cytosolic/nuclear delivery, thereby effecting gene expression modification.

### GENERAL DEFINITIONS

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, phosphorylation, glycosylation, sulfatation, sumoylation, prenylation, ubiquitination, etc.). For further clarity, protein/polypeptide/peptide modifications are envisaged so long as the modification does not destroy the cargo transduction activity of the shuttle agents described herein. For example, shuttle agents described herein may be linear or circular, may be synthesized with one or more D- or L-amino acids, and/or may be conjugated to a fatty acid (e.g., at their N terminus). Shuttle agents described herein may also have at least one amino acid being replaced with a corresponding synthetic amino acid having a side chain of similar physiochemical properties (e.g., structure, hydrophobicity, or charge) as the amino acid being replaced.

As used herein, a “domain” or “protein domain” generally refers to a part of a protein having a particular functionality or function. Some domains conserve their function when separated from the rest of the protein, and thus can be used in a modular fashion. The modular characteristic of many protein domains can provide flexibility in terms of their placement within the shuttle agents of the present description. However, some domains may perform better when engineered at certain positions of the shuttle agent (e.g., at the N- or C-terminal region, or therebetween). The position of the domain within its endogenous protein is sometimes an indicator of where the domain should be engineered within the shuttle agent and of what type/length of linker should be used. Standard recombinant DNA techniques can be used by the skilled person to manipulate the placement and/or number of the domains within the shuttle agents of the present description in view of the present disclosure. Furthermore, assays disclosed herein, as well as others known in the art, can be used to assess the functionality of each of the domains within the context of the shuttle agents (e.g., their ability to facilitate cell penetration across the plasma membrane, endosome escape, and/or access to the cytosol). Standard methods can also be used to assess whether the domains of the shuttle agent affect the activity of the cargo to be delivered intracellularly. In this regard, the expression “operably linked” as used herein refers to the ability of the domains to carry out their intended function(s) (e.g., cell penetration, endosome escape, and/or subcellular targeting) within the context of the shuttle agents of the present description. For greater clarity, the expression “operably linked” is meant to define a functional connection between two or more domains without being limited to a particular order or distance between same.

As used herein, the term “synthetic” used in expressions such as “synthetic peptide”, synthetic peptide shuttle agent”, or “synthetic polypeptide” is intended to refer to non-naturally occurring molecules that can be produced in vitro (e.g., synthesized chemically and/or produced using recombinant DNA technology). The purities of various synthetic preparations may be assessed by, for example, high-performance liquid chromatography analysis and mass spectroscopy. Chemical synthesis approaches may be advantageous over cellular expression systems (e.g., yeast or bacteria protein expression systems), as they may preclude the need for extensive recombinant protein purification steps (e.g., required for clinical use). In contrast, longer synthetic polypeptides may be more complicated and/or costly to produce via chemical synthesis approaches and such polypeptides may be more advantageously produced using cellular expression systems. In some embodiments, the peptides or shuttle agents of the present description may be chemically synthesized (e.g., solid- or liquid phase peptide synthesis), as opposed to expressed from a recombinant host cell. In some embodiments, the peptides or shuttle agent of the present description may lack an N-terminal methionine residue. A person of skill in the art may adapt a synthetic peptide or shuttle agent of the present description by using one or more modified amino acids (e.g., non-naturally-occurring amino acids), or by chemically modifying the synthetic peptide or shuttle agent of the present description, to suit particular needs of stability or other needs.

As used herein, the term “independent” is generally intended refer to molecules or agents which are not covalently bound to one another, or that may be transiently covalently linked via a cleavable bond such that the molecules or agents (e.g., shuttle agent and cargo) detach from one another through cleavage of the bond following administration (e.g., when exposed to the reducing cellular environment, and/or but prior to, simultaneously with, or shortly after being delivered intracellularly). For example, the expression “independent cargo” is intended to refer to a cargo to be delivered intracellularly (transduced) that is not covalently bound (e.g., not fused) to a shuttle agent of the present description at the time of transduction across the plasma membrane. In some aspects, having shuttle agents that are independent of (not fused to) a cargo may be advantageous by providing increased shuttle agent versatility—e.g., being able to readily vary the ratio of shuttle agent to cargo (as opposed to being limited to a fixed ratio in the case of a covalent linkage between the shuttle agent and cargo). In some aspects, covalently linking a shuttle agent to its cargo via a cleavable bond such that they detach from one another upon contact with target cells may be advantageous from a manufacturing and/or regulatory perspective.

As used herein, the expression “is or is from” or “is from” comprises functional variants of a given protein or peptide (e.g., a shuttle agent described herein) or domain thereof (e.g., CPD or ELD), such as conservative amino acid substitutions, deletions, modifications, as well as variants or function derivatives, which do not abrogate the activity of the protein domain.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the intracellular delivery in HeLa cells of PMO-FITC cargo via first generation “domain-based” and rationally-designed synthetic peptide shuttles agents, as assessed by flow cytometry. Results are means calculated from experiments performed at least in duplicate.

FIG. 2 shows the ability of the synthetic peptide shuttle agent FSD10 to transduce antisense PMO to the cytosol of HeLa cells, thereby enabling knock-down of GFP gene expression, as assessed by flow cytometry. Results are means calculated from experiments performed at least in duplicate.

FIG. 3 shows the ability of the synthetic peptide shuttle agent FSD250 to transduce antisense PMOs targeting Wnt1 and Gli1 for knock-down of Gli1 protein expression in DU145 cells, as assessed by Western blot. Results are means calculated from experiments performed at least in duplicate.

FIG. 4 shows the ability of the synthetic peptide shuttle agent FSD250 to transduce antisense PMOs targeting Gli1 for knock-down of Gli1 protein expression in DU145 cells, as assessed by Western blot. Results are means calculated from experiments performed at least in duplicate.

FIG. 5 shows the results of a large-scale screening of candidate peptide shuttle agents for propidium iodide (PI) and GFP-NLS transduction activity. Results are means calculated from experiments performed at least in duplicate.

FIG. 6 shows the results of a further screening of candidate peptide shuttle agents for propidium iodide (PI) and/or GFP-NLS transduction activity. Results are means calculated from experiments performed at least in duplicate.

FIG. 7 shows the ability of the synthetic peptide shuttle agent FSD250 to transduce antisense PMO and PNA in HeLa cells. FIG. 7A shows the results of intracellular delivery by flow cytometry. FIG. 7B shows the results of the viability of the cells by flow cytometry. Results are means calculated from experiments performed at least in duplicate.

FIG. 8 shows the results of the inhibitory effect of naked DNA (FIG. 8A) and RNA (sgRNA; FIG. 8B) on intracellular delivery of PMO by the synthetic peptide shuttle agent FSD250 by flow cytometry.

FIG. 9 shows the ability of second-generation synthetic peptide shuttle agents FSD10 and FSD250 to transduce antisense PMO in RH-30 cells, in comparison to His-CM18-PTD4 (first-generation shuttle agent). FIG. 9A shows the results of intracellular delivery by flow cytometry. FIG. 9B shows the results of the viability of the cells by flow cytometry. Results are means calculated from experiments performed at least in duplicate.

FIG. 10 shows the results of Gli1 knockdown in RH-30 cells after transduction of PMO-Gli1 with the synthetic peptide shuttle agent FSD250, in comparison with VivoPMO-Gli1. FIG. 10A shows the results of a western blot for Gli1 and GAPDH (control). FIG. 10B shows the results of the densitometry scanning analysis of the corresponding western blot of FIG. 10A.

FIG. 11 shows the ability of the synthetic peptide shuttle agent FSD396 to transduce antisense PMO in HeLa cells, in comparison to Endoporter™. Representative immunofluorescence microscopy images of untreated (FIG. 11A), PMO-FITC treated (FIG. 11B), PMO-FITC+FSD396 (FIG. 11C), and PMO-FITC+Endoporter (FIG. 11D) are shown.

FIG. 12 shows the ability of four different PMOs targeting Gli1 to knockdown Gli1 in Human DU145 cells, only after transduction with the synthetic peptide shuttle agent FSD250. A representative Western Blot for Gli1 and actinin (control) and corresponding densitometry scanning analysis is shown.

FIG. 13 shows the results of PMO-Gli1 transduction into basal cell carcinoma-type tumor explants with the synthetic peptide shuttle agent FSD250. Representative fluorescence microscopy images of Cy5 (left), Cy5+DAPI (nuclear staining; middle), and Cy5+DAPI+Nomarski (i.e., differential interference microscopy [DIC]) (right) are shown after treatment with PMO-Gli1-Cy5 and FSD250, or PMO-Gli1-Cy5 alone.

### SEQUENCE LISTING

This application contains a Sequence Listing in computer readable form created Oct. 15, 2021. The computer readable form is incorporated herein by reference.

| SEQ ID NO: | Description |
|---|---|
| 1 | CM18-Penetratin-cys |
| 2 | TAT-KALA |
| 3 | His-CM18-PTD4 |
| 4 | His-LAH4-PTD4 |
| 5 | PTD4-KALA |
| 6 | EB1-PTD4 |
| 7 | His-CM18-PTD4-6Cys |
| 8 | CM18-PTD4 |
| 9 | CM18-PTD4-6His |
| 10 | His-CM18-PTD4-His |
| 11 | TAT-CM18 |
| 12 | FSD5 |
| 13 | FSD10 |
| 14 | FSD12 |
| 15 | FSD18 |
| 16 | FSD19 |
| 17 | FSD21 |
| 18 | FSD23 |
| 19 | FSD120 |
| 20 | FSD127 |
| 21 | FSD129 |
| 22 | FSD131 |
| 23 | FSD134 |
| 24 | FSD146 |
| 25 | FSD155 |
| 26 | FSD156 |
| 27 | FSD157 |
| 28 | FSD159 |
| 29 | FSD162 |
| 30 | FSD168 |
| 31 | FSD173 |
| 32 | FSD174 |
| 33 | FSD194 |
| 34 | FSD220 |
| 35 | FSD250 |
| 36 | FSD250D |
| 37 | FSD253 |
| 38 | FSD258 |
| 39 | FSD262 |
| 40 | FSD263 |
| 41 | FSD264 |
| 42 | FSD265 |
| 43 | FSD268 |
| 44 | FSD286 |
| 45 | FSD271 |
| 46 | FSD272 |
| 47 | FSD273 |
| 48 | FSD276 |
| 49 | FSD268 Cyclic Amide |
| 50 | FSD268 Cyclic Disulfide |
| 51 | FSD10 Scramble |
| 52 | FSD268 Scramble |
| 53 | FSD174 Scramble |
| 54 | FSN3 |
| 55 | FSN4 |
| 56 | FSN7 |
| 57 | FSN8 |
| 58 | FSD117 |
| 59 | FSD118 |
| 60 | FSD119 |
| 61 | FSD121 |
| 62 | FSD122 |
| 63 | FSD123 |
| 64 | FSD124 |
| 65 | FSD125 |
| 66 | FSD126 |
| 67 | FSD127 |
| 68 | FSD128 |
| 69 | FSD130 |
| 70 | FSD132 |
| 71 | FSD133 |
| 72 | FSD135 |
| 73 | FSD137 |
| 74 | FSD138 |
| 75 | FSD139 |
| 76 | FSD140 |
| 77 | FSD141 |
| 78 | FSD142 |
| 79 | FSD143 |
| 80 | FSD144 |
| 81 | FSD145 |
| 82 | FSD147 |
| 83 | FSD148 |
| 84 | FSD149 |
| 85 | FSD150 |
| 86 | FSD151 |
| 87 | FSD152 |
| 88 | FSD153 |
| 89 | FSD154 |
| 90 | FSD158 |
| 91 | FSD160 |
| 92 | FSD161 |
| 93 | FSD163 |
| 94 | FSD164 |
| 95 | FSD165 |
| 96 | FSD166 |
| 97 | FSD167 |
| 98 | FSD169 |
| 99 | FSD170 |
| 100 | FSD171 |
| 101 | FSD172 |
| 102 | FSD175 |
| 103 | FSD176 |
| 104 | FSD177 |
| 105 | FSD178 |
| 106 | FSD179 |
| 107 | FSD180 |
| 108 | FSD181 |
| 109 | FSD182 |
| 110 | FSD183 |
| 111 | FSD184 |
| 112 | FSD185 |
| 113 | FSD186 |
| 114 | FSD187 |
| 115 | FSD188 |
| 116 | FSD189 |
| 117 | FSD190 |
| 118 | FSD191 |
| 119 | FSD192 |
| 120 | FSD193 |
| 121 | FSD195 |
| 122 | FSD196 |
| 123 | FSD197 |
| 124 | FSD198 |
| 125 | FSD199 |
| 126 | FSD200 |
| 127 | FSD201 |
| 128 | FSD202 |
| 129 | FSD203 |
| 130 | FSD204 |
| 131 | FSD205 |
| 132 | FSD206 |
| 133 | FSD207 |
| 134 | FSD208 |
| 135 | FSD209 |
| 136 | FSD210 |
| 137 | FSD211 |
| 138 | FSD212 |
| 139 | FSD213 |
| 140 | FSD214 |
| 141 | FSD215 |
| 142 | FSD216 |
| 143 | FSD217 |
| 144 | FSD218 |
| 145 | FSD219 |
| 146 | FSD221 |
| 147 | FSD222 |
| 148 | FSD223 |
| 149 | FSD224 |
| 150 | FSD225 |
| 151 | FSD226 |
| 152 | FSD227 |
| 153 | FSD228 |
| 154 | FSD229 |
| 155 | FSD230 |
| 156 | FSD231 |
| 157 | FSD232 |
| 158 | FSD233 |
| 159 | FSD234 |
| 160 | FSD235 |
| 161 | FSD236 |
| 162 | FSD237 |
| 163 | FSD238 |
| 164 | FSD239 |
| 165 | FSD240 |
| 166 | FSD241 |
| 167 | FSD243 |
| 168 | FSD244 |
| 169 | FSD246 |
| 170 | FSD247 |
| 171 | FSD248 |
| 172 | FSD250 Scramble |
| 173 | FSD250E |
| 174 | FSD251 |
| 175 | FSD254 |
| 176 | FSD255 |
| 177 | FSD256 |
| 178 | FSD257 |
| 179 | FSD259 |
| 180 | FSD260 |
| 181 | FSD261 |
| 182 | FSD266 |
| 183 | FSD267 |
| 184 | FSD269 |
| 185 | FSD270 |
| 186 | FSD274 |
| 187 | FSD275 |
| 188 | FSD276 |
| 189 | FSD277 |
| 190 | FSD278 |
| 191 | FSD279 |
| 192 | FSD280 |
| 193 | FSD281 |
| 194 | FSD282 |
| 195 | FSD283 |
| 196 | FSD284 |
| 197 | FSD285 |
| 198 | FSD287 |
| 199 | FSD288 |
| 200 | FSD289 |
| 201 | FSD290 |
| 202 | FSD291 |
| 203 | FSD292 |
| 204 | FSD293 |
| 205 | FSD294 |
| 206 | FSD295 |
| 207 | FSD296 |
| 208 | FSD297 |
| 209 | FSD298 |
| 210 | FSD299 |
| 211 | FSD300 |
| 212 | FSD301 |
| 213 | FSD302 |
| 214 | FSD303 |
| 215 | FSD304 |
| 216 | FSD305 |
| 217 | FSD306 |
| 218 | FSD307 |
| 219 | FSD308 |
| 220 | FSD309 |
| 221 | FSD310 |
| 222 | FSD311 |
| 223 | FSD312 |
| 224 | FSD313 |
| 225 | FSD314 |
| 226 | FSD315 |
| 227 | FSD316 |
| 228 | FSD317 |
| 229 | FSD318 |
| 230 | FSD319 |
| 231 | FSD320 |
| 232 | FSD321 |
| 233 | FSD322 |
| 234 | FSD323 |
| 235 | FSD324 |
| 236 | FSD325 |
| 237 | FSD326 |
| 238 | FSD327 |
| 239 | FSD328 |
| 240 | FSD330 |
| 241 | FSD331 |
| 242 | FSD332 |
| 243 | FSD333 |
| 244 | FSD334 |
| 245 | FSD335 |
| 246 | FSD336 |
| 247 | FSD337 |
| 248 | FSD338 |
| 249 | FSD339 |
| 250 | FSD340 |
| 251 | FSD341 |
| 252 | FSD342 |
| 253 | FSD343 |
| 254 | FSD344 |
| 255 | FSD345 |
| 256 | FSD346 |
| 257 | FSD347 |
| 258 | FSD348 |
| 259 | FSD349 |
| 260 | FSD350 |
| 261 | FSD351 |
| 262 | FSD352 |
| 263 | FSD353 |
| 264 | FSD354 |
| 265 | FSD355 |
| 266 | FSD356 |
| 267 | FSD357 |
| 268 | FSD358 |
| 269 | FSD359 |
| 270 | FSD360 |
| 271 | FSD361 |
| 272 | FSD362 |
| 273 | FSD363 |
| 274 | FSD364 |
| 275 | FSD365 |
| 276 | FSD366 |
| 277 | FSD367 |
| 278 | FSD368 |
| 279 | FSD369 |
| 280 | FSD370 |
| 281 | FSD371 |
| 282 | FSD372 |
| 283 | FSD373 |
| 284 | FSD374 |
| 285 | FSD375 |
| 286 | FSD376 |
| 287 | FSD377 |
| 288 | FSD378 |
| 289 | FSD379 |
| 290 | FSD381 |
| 291 | FSD382 |
| 292 | FSD383 |
| 293 | FSD384 |
| 294 | FSD385 |
| 295 | FSD386 |
| 296 | FSD387 |
| 297 | FSD388 |
| 298 | FSD389 |
| 299 | FSD390 |
| 300 | FSD391 |
| 301 | FSD392 |
| 302 | FSD393 |
| 303 | FSD394 |
| 304 | FSD395 |
| 305 | FSD396 |
| 306 | FSD397 |
| 307 | FSD398 |
| 308 | FSD399 |
| 309 | FSD400 |
| 310 | FSD401 |
| 311 | FSD402 |
| 312 | FSD403 |
| 313 | FSD404 |
| 314 | FSD406 |
| 315 | FSD407 |
| 316 | FSD408 |
| 317 | FSD409 |
| 318 | FSD410 |
| 319 | FSD411 |
| 320 | FSD412 |
| 321 | FSD413 |
| 322 | FSD414 |
| 323 | FSD415 |
| 324 | FSD416 |
| 325 | FSD417 |
| 326 | FSD418 |
| 327 | FSD419 |
| 328 | FSD421 |
| 329 | FSD422 |
| 330 | FSD423 |
| 331 | FSD424 |
| 332 | FSD425 |
| 333 | FSD426 |
| 334 | FSD427 |
| 335 | FSD428 |
| 336 | FSD429 |
| 337 | FSD430 |
| 338 | FSD431 |
| 339 | FSD432 |
| 340 | FSD433 |
| 341 | FSD434 |
| 342 | FSD435 |
| 343 | FSD436 |
| 344 | FSD438 |
| 345 | PMO-GFP |
| 346 | PMO-Gli1 |
| 347 | PMO-Wnt1 |
| 348 | PMO-FITC |
| 349 | siRNA-GFP |
| 350 | siRNA-Gli1 |
| 351 | PTD4 |
| 352 | TAT |
| 353 | CM18 |
| 354 | KALA |
| 355 | Penetratin |
| 356 | FSD92 |
| 357 | FSD10-15 |
| 358 | FSD418-12-2 |
| 359 | FSD418-15 |
| 360 | FSD440 |
| 361 | FSD441 |
| 362 | FSD443 |
| 363 | FSD445 |
| 364 | FSD446 |
| 365 | Gli target of PMO-Gli1_Opt |
| 366 | Gli target of PMO-Gli1_Opt1 |
| 367 | Gli target of PMO-Gli1_Opt2 |
| 368 | Gli target of PMO-Gli1_Opt3 |

### DETAILED DESCRIPTION

In some aspects, described herein are compositions and methods for non-anionic polynucleotide analog cargo transduction. The methods generally comprise contacting target eukaryotic cells with a composition comprising the non-anionic polynucleotide analog cargo and a synthetic peptide shuttle agent that is independent from, or is not covalently linked to, the non-anionic polynucleotide analog cargo, wherein synthetic peptide shuttle agent increases cytosolic/nuclear delivery of said non-anionic polynucleotide analog cargo in eukaryotic cells.

### Non-Anionic Polynucleotide Analog Cargoes

In some embodiments, the non-anionic polynucleotide analog cargoes may be charge-neutral or cationic antisense synthetic oligonucleotides (ASOs). In some embodiments, the ASO may be a charge-neutral or cationic splice-switching oligonucleotide (SSO). In some embodiments, the non-anionic polynucleotide analog cargo may be a charge-neutral polynucleotide analog cargo having a phosphorodiamidate backbone, an amide (e.g., peptide) backbone, a methylphosphonate backbone, a neutral phosphotriester backbone, a sulfone backbone, or a triazole backbone. In some embodiments, the non-anionic polynucleotide analog cargo may be a cationic polynucleotide analog cargo having an aminoalkylated phosphoramidate backbone, a guanidinium backbone, an S-methylthiourea backbone, or a nucleosyl amino acid (NAA) backbone. In some embodiments, the non-anionic polynucleotide analog cargo may be a phosphorodiamidate morpholino oligomer (PMO), a peptide nucleic acid (PNA), a methylphosphonate oligomer, or a short interfering ribonucleic neutral oligonucleotide (siRNN). In some embodiments, the non-anionic polynucleotide analog cargo may be a 5- to 50-mer, a 5-mer to 75-mer, or a 5-mer to 100-mer. In some embodiments, the non-anionic polynucleotide analog cargo is not covalently linked to a cell-penetrating peptide, octa-guanidine dendrimer, or other intracellular delivery moiety. In some embodiments, the non-anionic polynucleotide analog cargo is cell membrane-impermeable or has low membrane permeability (e.g., due to the physicochemical properties of the cargo precluding it from freely diffusing across the cell membrane), wherein the peptide shuttle agents described herein facilitate or increase its intracellular delivery and/or access to the cytosol/nucleus. In some embodiments, the non-anionic polynucleotide analog cargo may be a cargo that is cell membrane-permeable, wherein peptide shuttle agents described herein nevertheless increase its intracellular delivery and/or access to the cytosol. In some embodiments, peptide shuttle agents described herein may reduce the amount or concentration of the cargo that is required to be administered to achieve its intended biological effect, as compared to administration of the cargo alone.

In some embodiments, the non-anionic polynucleotide analog cargo may be a drug for treating any disease or condition that modifies gene expression of a therapeutically relevant target RNA. In some embodiments, the non-anionic polynucleotide analog cargo may be a drug for treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis).

In some embodiments, the non-anionic polynucleotide analog cargoes described herein may be a splice switching oligonucleotide (SSO), for example for correcting or modifying the splicing of a therapeutically relevant target mRNA. In some embodiments, the target mRNA may be the cystic fibrosis transmembrane conductance regulator (CFTR) and the composition or method described herein may be for the treatment of cystic fibrosis (e.g., via administration to the lungs of a cystic fibrosis subject). In this regard, synthetic peptide shuttle agents have been shown to enable efficient delivery of recombinant protein cargoes to refractory airway epithelial cells (Krishnamurthy et al., 2018).

In some embodiments, the non-anionic polynucleotide analog cargoes described herein are not covalently linked to a cell-penetrating or cationic peptide, an octa-guanidine dendrimer, or other intracellular delivery moiety. Such conventional delivery strategies, which have been employed for example in peptide-conjugated phosphorodiamidate morpholino oligomers (PPMOs) and Vivo-Morpholinos, add a further layer of complexity to the synthesis process of PMOs. In contrast, the synthetic peptide shuttle agents described herein can advantageously transduce unmodified or “naked” non-anionic polynucleotide analog cargoes, greatly facilitating manufacture and formulation.

### Rational Design Parameters and Peptide Shuttle Agents

In some aspects, the shuttle agents described herein may be a peptide having transduction activity for non-anionic polynucleotide analog cargoes, proteinaceous cargoes, or both in target eukaryotic cells. In some embodiments, the shuttle agents described herein preferably satisfy one or more or any combination of the following fifteen rational design parameters.

(1) In some embodiments, the shuttle agent is a peptide at least 12, 13, 14, 15, 16, 17, 18, 19, or 20 amino acids in length. For example, the peptide may comprise a minimum length of 17, 18, 19, 20, 21, 22, 23, 24, 25, 26, 27, 28, 29, or 30 amino acid residues, and a maximum length of 35, 36, 37, 38, 39, 40, 41, 42, 43, 44, 45, 46, 47, 48, 49, 50, 51, 52, 53, 54, 55, 60, 65, 70, 75, 80, 85, 90, 95, 100, 105, 110, 115, 120, 125, 130, 135, 140, 145, or 150 amino acid residues. In some embodiments, shorter peptides (e.g., in the 17-50 or 20-50 amino acid range) may be particularly advantageous because they may be more easily synthesized and purified by chemical synthesis approaches, which may be more suitable for clinical use (as opposed to recombinant proteins that must be purified from cellular expression systems). While numbers and ranges in the present description are often listed as multiples of 5, the present description should not be so limited. For example, the maximum length described herein should be understood as also encompassing a length of 56, 57, 58 . . . 61, 62, etc., in the present description, and that their non-listing herein is only for the sake of brevity. The same reasoning applies to the % of identities listed herein.
(2) In some embodiments, the peptide shuttle agent comprises an amphipathic alpha-helical motif at neutral pH. As used herein, the expression “alpha-helical motif” or “alpha-helix”, unless otherwise specified, refers to a right-handed coiled or spiral conformation (helix) having angle of rotation between consecutive amino acids of 100 degrees and/or an alpha-helix having 3.6 residues per tum. As used herein, the expression “comprises an alpha-helical motif” or “an amphipathic alpha-helical motif” and the like, refers to the three-dimensional conformation that a peptide (or segment of a peptide) of the present description is predicted to adopt when in a biological setting based on the peptide's primary amino acid sequence, regardless of whether the peptide actually adopts that conformation when used in cells as a shuttle agent. Furthermore, the peptides of the present description may comprise one or more alpha-helical motifs in different locations of the peptide. For example, the shuttle agent FSDS in WO/2018/068135 is predicted to adopt an alpha-helix over the entirety of its length (see FIG. 49C of WO/2018/068135), while the shuttle agent FSD18 of WO/2018/068135 is predicted to comprise two separate alpha-helices towards the N and C terminal regions of the peptide (see FIG. 49D of WO/2018/068135). In some embodiments, the shuttle agents of the present description are not predicted to comprise a beta-sheet motif, for example as shown in FIGS. 49E and 49F of WO/2018/068135. Methods of predicting the presence of alpha-helices and beta-sheets in proteins and peptides are well known in the art. For example, one such method is based on 3D modeling using PEP-FOLD™, an online resource for de novo peptide structure prediction (http://bioserv.rpbs.univ-paris-diderot.fr/services/PEP-FOLD/) (Lamiable et al., 2016; Shen et al., 2014; Thévenet et al., 2012). Other methods of predicting the presence of alpha-helices in peptides and protein are known and readily available to the skilled person.

As used herein, the expression “amphipathic” refers to a peptide that possesses both hydrophobic and hydrophilic elements (e.g., based on the side chains of the amino acids that comprise the peptide). For example, the expression “amphipathic alpha helix” or “amphipathic alpha-helical motif” refers to a peptide predicted to adopt an alpha-helical motif having a non-polar hydrophobic face and a polar hydrophilic face, based on the properties of the side chains of the amino acids that form the helix.

(3) In some embodiments, peptide shuttle agents of the present description comprise an amphipathic alpha-helical motif having a positively-charged hydrophilic outer face, such as one that is rich in R and/or K residues. As used herein, the expression “positively-charged hydrophilic outer face” refers to the presence of at least three lysine (K) and/or arginine (R) residues clustered to one side of the amphipathic alpha-helical motif, based on alpha-helical wheel projection (e.g., see FIG. 49A, left panel of WO/2018/068135). Such helical wheel projections may be prepared using a variety of programs, such as the online helical wheel projection tool created by Don Armstrong and Raphael Zidovetzki. (e.g., available at: https://www.donarmstrong.com/cgi-bin/wheel.pl) or the online tool developed by Mól et al., 2018 (e.g., available at http://lbqp.unb.br/NetWheels/). In some embodiments, the amphipathic alpha-helical motif may comprise a positively-charged hydrophilic outer face that comprises: (a) at least two, three, or four adjacent positively-charged K and/or R residues upon helical wheel projection; and/or (b) a segment of six adjacent residues comprising three to five K and/or R residues upon helical wheel projection, based on an alpha helix having angle of rotation between consecutive amino acids of 100 degrees and/or an alpha-helix having 3.6 residues per turn.

In some embodiments, peptide shuttle agents of the present description comprise an amphipathic alpha-helical motif comprising a hydrophobic outer face, the hydrophobic outer face comprising: (a) at least two adjacent L residues upon helical wheel projection; and/or (b) a segment often adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per tum.

(4) In some embodiments, peptide shuttle agents of the present description comprise an amphipathic alpha-helical motif having a highly hydrophobic core composed of spatially adjacent highly hydrophobic residues (e.g., L, I, F, V, W, and/or M). In some embodiments, the highly hydrophobic core may consist of spatially adjacent L, I, F, V, W, and/or M amino acids representing 12 to 50% of the amino acids of the peptide, calculated while excluding any histidine-rich domains (see below), based on an open cylindrical representation of the alpha-helix having 3.6 residues per turn, as shown for example in FIG. 49A, right panel of WO/2018/068135. In some embodiments, the highly hydrophobic core may consist of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the peptide. More particularly, highly hydrophobic core parameter may be calculated by first arranging the amino acids of the peptide in an opened cylindrical representation, and then delineating an area of contiguous highly hydrophobic residues (L, I, F, V, W, M), as shown in FIG. 49A, right panel of WO/2018/068135. The number of highly hydrophobic residues comprised in this delineated highly hydrophobic core is then divided by the total amino acid length of the peptide, excluding any histidine-rich domains (e.g., N- and/or C-terminal histidine-rich domains). For example, for the peptide shown in FIG. 49A of WO/2018/068135, there are 8 residues in the delineated highly hydrophobic core, and 25 total residues in the peptide (excluding the terminal 12 histidines). Thus, the highly hydrophobic core is 32% ( 8/25).
(5) Hydrophobic moment relates to a measure of the amphiphilicity of a helix, peptide, or part thereof, calculated from the vector sum of the hydrophobicities of the side chains of the amino acids (Eisenberg et al., 1982). An online tool for calculating the hydrophobic moment of a polypeptide is available from: http://rzlab.ucr.edu/scripts/wheel/wheel.cgi. A high hydrophobic moment indicates strong amphiphilicity, while a low hydrophobic moment indicates poor amphiphilicity. In some embodiments, peptide shuttle agents of the present description may consist of or comprise a peptide or alpha-helical domain having have a hydrophobic moment (μ) of 3.5 to 11. In some embodiments, the shuttle agent may be a peptide comprising an amphipathic alpha-helical motif having a hydrophobic moment between a lower limit of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.8, 10.9, or 11.0. In some embodiments, the shuttle agent may be a peptide having a hydrophobic moment between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.2, 10.3, 10.4, or 10.5. In some embodiments, the hydrophobic moment is calculated excluding any histidine-rich domains that may be present in the peptide.
(6) In some embodiments, peptide shuttle agents of the present description may have a predicted net charge of at least +3 or +4 at physiological pH, calculated from the side chains of K, R, D, and E residues. For example, the net charge of the peptide may be at least +5, +6, +7, at least +8, at least +9, at least +10, at least +11, at least +12, at least +13, at least +14, or at least +15 at physiological pH. These positive charges are generally conferred by the greater presence of positively-charged lysine and/or arginine residues, as opposed to negatively charged aspartate and/or glutamate residues.
(7) In some embodiments, peptide shuttle agents of the present description may have a predicted isoelectric point (pI) of 8 to 13, preferably from 10 to 13. Programs and methods for calculating and/or measuring the isoelectric point of a peptide or protein are known in the art. For example, pI may be calculated using the Prot Param software available at: http://web.expasy.org/protparam/
(8) In some embodiments, peptide shuttle agents of the present description may be composed of 35 to 65% of hydrophobic residues (A, C, G, I, L, M, F, P, W, Y, V). In particular embodiments, the peptide shuttle agents may be composed of 36% to 64%, 37% to 63%, 38% to 62%, 39% to 61%, or 40% to 60% of any combination of the amino acids: A, C, G, I, L, M, F, P, W, Y, and V.
(9) In some embodiments, peptide shuttle agents of the present description may be composed of 0 to 30% of neutral hydrophilic residues (N, Q, S, T). In particular embodiments, the peptide shuttle agents may be composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T.
(10) In some embodiments, peptide shuttle agents of the present description may be composed of 35 to 85% of the amino acids A, L, K and/or R. In particular embodiments, the peptide shuttle agents may be composed of 36% to 80%, 37% to 75%, 38% to 70%, 39% to 65%, or 40% to 60% of any combination of the amino acids: A, L, K, or R.
(11) In some embodiments, peptide shuttle agents of the present description may be composed of 15 to 45% of the amino acids A and/or L, provided there being at least 5% of L in the peptide. In particular embodiments, the peptide shuttle agents may be composed of 15% to 40%, 20% to 40%, 20 to 35%, or 20 to 30% of any combination of the amino acids: A and L, provided there being at least 5% of L in the peptide.
(12) In some embodiments, peptide shuttle agents of the present description may be composed of 20 to 45% of the amino acids K and/or R. In particular embodiments, the peptide shuttle agents may be composed of 20% to 40%, 20 to 35%, or 20 to 30% of any combination of the amino acids: K and R.
(13) In some embodiments, peptide shuttle agents of the present description may be composed of 0 to 10% of the amino acids D and/or E. In particular embodiments, the peptide shuttle agents may be composed of 5 to 10% of any combination of the amino acids: D and E.
(14) In some embodiments, the absolute difference between the percentage of A and/or L and the percentage of K and/or R in the peptide shuttle agent may be less than or equal to 10%. In particular embodiments, the absolute difference between the percentage of A and/or L and the percentage of K and/or R in the peptide shuttle agent may be less than or equal to 9%, 8%, 7%, 6%, or 5%.
(15) In some embodiments, peptide shuttle agents of the present description may be composed of 10% to 45% of the amino acids Q, Y, W, P, I, S, G, V, F, E, D, C, M, N, T, or H (i.e., not A, L, K, or R). In particular embodiments, the peptide shuttle agents may be composed of 15 to 40%, 20% to 35%, or 20% to 30% of any combination of the amino acids: Q, Y, W, P, I, S, G, V, F, E, D, C, M, N, T, and H.

In some embodiments, peptide shuttle agents of the present description respect at least one, at least two, at least three, at least four, at least five, at least six, at least seven, at least eight, at least nine, at least ten, at least eleven, at least twelve, at leave thirteen, at least fourteen, or all of parameters (1) to (15) described herein. In particular embodiments, peptide shuttle agents of the present description respect all of parameters (1) to (3), and at least six, at least seven, at least eight, at least nine, at least ten, at least eleven, or all of parameters (4) to (15) described herein.

In some embodiments, where a peptide shuttle agent of the present description comprises only one histidine-rich domain, the residues of the one histidine-rich domain may be included in the calculation/assessment of parameters (1) to (15) described herein. In some embodiments, where a peptide shuttle agent of the present description comprises more than one histidine-rich domain, only the residues of one of the histidine-rich domains may be included in the calculation/assessment of parameters (1) to (15) described herein. For example, where a peptide shuttle agent of the present description comprises two histidine-rich domains: a first histidine-rich domain towards the N terminus, and a second histidine-rich domain towards the C terminus, only the first histidine-rich domain may be included in the calculation/assessment of parameters (1) to (15) described herein.

In some embodiments, a machine-learning or computer-assisted design approach may be implemented to generate peptides that respect one or more of parameters (1) to (15) described herein. Some parameters, such as parameters (1) and (5)-(15), may be more amenable to implementation in a computer-assisted design approach, while structural parameters, such as parameters (2), (3) and (4), may be more amenable to a manual design approach. Thus, in some embodiments, peptides that respect one or more of parameters (1) to (15) may be generated by combining computer-assisted and manual design approaches. For example, multiple sequence alignment analyses of a plurality of peptides shown herein (and others) to function as effective shuttle agents revealed the presence of some consensus sequences — i.e., commonly found patterns of alternance of hydrophobic, cationic, hydrophilic, alanine and glycine amino acids. The presence of these consensus sequences are likely to give rise to structural parameters (2), (3) and (4) being respected (i.e., amphipathic alpha-helix formation, a positively-charged face, and a highly hydrophobic core of 12%-50%). Thus, these and other consensus sequences may be employed in machine-learning and/or computer-assisted design approaches to generate peptides that respect one or of parameters (1)-(15).

Accordingly, in some embodiments, peptide shuttle agents described herein may comprise or consist of the amino acid sequence of:
(a) [X1]-[X2]-[linker]-[X3]-[X4] (Formula 1);
(b) [X1]-[X2]-[linker]-[X4]-[X3] (Formula 2);
(c) [X2]-[X1]-[linker]-[X3]-[X4] (Formula 3);
(d) [X2]-[X1]-[linker]-[X4]-[X3] (Formula 4);
(e) [X3]-[X4]-[linker]-[X1]-[X2] (Formula 5);
(f) [X3]-[X4]-[linker]-[X2]-[X1] (Formula 6);
(g) [X4]-[X3]-[linker]-[X1]-[X2] (Formula 7);
(h) [X4]-[X3]-[linker]-[X2]-[X1] (Formula 8);
(i) [linker]-[X1]-[X2]-[linker] (Formula 9);
(j) [linker]-[X2]-[X1]-[linker] (Formula 10);
(k) [X1]-[X2]-[linker] (Formula 11);
(l) [X2]-[X1]-[linker] (Formula 12);
(m) [linker]-[X1]-[X2] (Formula 13);
(n) [linker]-[X2]-[X1] (Formula 14);
(o) [X1]-[X2] (Formula 15); or
(p) [X2]-[X1] (Formula 16),
   wherein:
[X1] is selected from: 2[Φ]-1[+]-2[Φ]-1[ζ]-1[+]-; 2[Φ]-1[+]-2[Φ]-2[+]-; 1[+]-1[Φ]-1[+]-2[Φ]-1[ζ]-1[+]-; and 1[+]-1[Φ]-1[+]-2[Φ]-2[+]-;
[X2] is selected from: -2[Φ]-1[+]-2[Φ]-2[ζ]-; -2[Φ]-1[+]-2[Φ]-2[+]-; -2[Φ]-1[+]-2[Φ]-1[+]-1[ζ]; -2[Φ]-1[+]-2[Φ]-1[ζ]-1[+]-; -2[Φ]-2[+]-1[Φ]-2[+]-; -2[Φ]-2[+]-1[Φ]-2[ζ]-; -2[Φ]-2[+]-1[Φ]-1[+]-1[ζ]-; and -2[Φ]-2[+]-1[Φ]-1[ζ]-1[+]-;
[X3] is selected from: -4[+]-A-; -3[+]-G-A-; -3[+]-A-A-; -2[+]-1[Φ]-1[+]-A-; -2[+]-1[Φ]-G-A-; -2[+]-1[Φ]-A-A-; or -2[+]-A-1[+]-A; -2[+]-A-G-A; -2[+]-A-A-A-; -1[Φ]-3[+]-A-; -1[Φ]-2[+]-G-A-; -1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[Φ]-1[+]-A-1[+]-A; -1[Φ]-1[+]-A-G-A; -1[Φ]-1[+]-A-A-A; -A-1[+]-A-1[+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1[ζ]-A-1[+]; -1[+]-1[ζ]-A-1[+]-A; -1[+]-1[ζ]-A-2[+]; -1[+]-1[ζ]-A-1[+]-1[ζ]-A-1[+]; -1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and
[linker] is selected from: -Gn-; -Sn-; -(GnSn)n-; -(GnSn)nGn-; -(GnSn)nSn-; -(GnSn)nGn(GnSn)n-; and -(GnSn)nSn(GnSn)n-;
   wherein: [Φ] is an amino acid which is: Leu, Phe, Trp, Ile, Met, Tyr, or Val, preferably Leu, Phe, Trp, or Ile; [+] is an amino acid which is: Lys or Arg; [ζ] is an amino acid which is: Gln, Asn, Thr, or Ser; A is the amino acid Ala; G is the amino acid Gly; S is the amino acid Ser; and n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4, or 1 to 3.

In some embodiments, peptide shuttle agents of the present description may comprise or consist of a peptide which is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364, or to the amino acid sequence of any one of SEQ ID NOs: 104, 105, 107, 108, 110-131, 133-135, 138, 140, 142, 145, 148, 151, 152, 169-242, and 243-10 242 as disclosed in WO/2018/068135, or a functional variant thereof. In some embodiments, peptide shuttle agents of the present description may comprise the amino acid sequence motifs of SEQ ID NOs: 158 and/or 159 of WO/2018/068135, which were found in each of peptides FSDS, FSD16, FSD18, FSD19, FSD20, FSD22, and FSD23. In some embodiments, peptide shuttle agents of the present description may comprise the amino acid sequence motif of SEQ ID NO: 158 of WO/2018/068135 operably linked to the amino acid sequence motif of SEQ ID NO: 159 of WO/2018/068135. As used herein, a “functional variant” refers to a peptide having cargo transduction activity, which differs from the reference peptide by one or more conservative amino acid substitutions. As used herein in the context of functional variants, a “conservative amino acid substitution” is one in which one amino acid residue is replaced with another amino acid residue having a similar side chain Families of amino acid residues having similar side chains have been well defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, phenylalanine, methionine, tryptophan, and optionally proline), beta-branched side chains (e.g., threonine, valine, isoleucine) and aromatic side chains (e.g., tyrosine, phenylalanine, tryptophan, histidine).

In some embodiments, peptide shuttle agents of the present description do not comprise one or more of the amino acid sequences of any one of SEQ ID NOs: 57-59, 66-72, or 82-102 of WO/2018/068135. In some embodiments, peptide shuttle agents of the present description do not comprise one or more of the amino acid sequences of any one of SEQ ID NOs: 104, 105, 107, 108, 110-131, 133-135, 138, 140, 142, 145, 148, 151, 152, 169-242, and 243-10 242 as disclosed in WO/2018/068135. Rather, in some embodiments, peptide shuttle agents of the present description may relate to variants of such previously described shuttle agent peptides, wherein the variants are further engineered for improved transduction activity (i.e., capable of more robustly transducing non-anionic polynucleotide analog cargoes).

In some embodiments, peptide shuttle agents of the present description may have a minimal threshold of transduction efficiency and/or cargo delivery score for a “surrogate” cargo as measured in a eukaryotic cell model system (e.g., an immortalized eukaryotic cell line) or in a model organism. The expression “transduction efficiency” refers to the percentage or proportion of a population of target cells into which a cargo of interest is delivered intracellularly, which can be determined for example by flow cytometry, immunofluorescence microscopy, and other suitable methods may be used to assess cargo transduction efficiency (e.g., as described in WO/2018/068135). In some embodiments, transduction efficiency may be expressed as a percentage of cargo-positive cells. In some embodiments, transduction efficiency may be expressed as a fold-increase (or fold-decrease) over a suitable negative control assessed under identical conditions except for in the absence of cargo and shuttle agent (“no treatment”; NT) or in the absence of shuttle agent (“cargo alone”).

In some embodiments, the shuttle agents described herein comprises or consists of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 (e.g., calculated excluding any linker domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 by only conservative amino acid substitutions (e.g., by no more than no more than 1, 2, 3, 4, 5, 6, 7, 8, 9, or 10 conservative amino acid substitutions, preferably excluding any linker domains), wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q; or
(v) any combination of (i) to (iv).

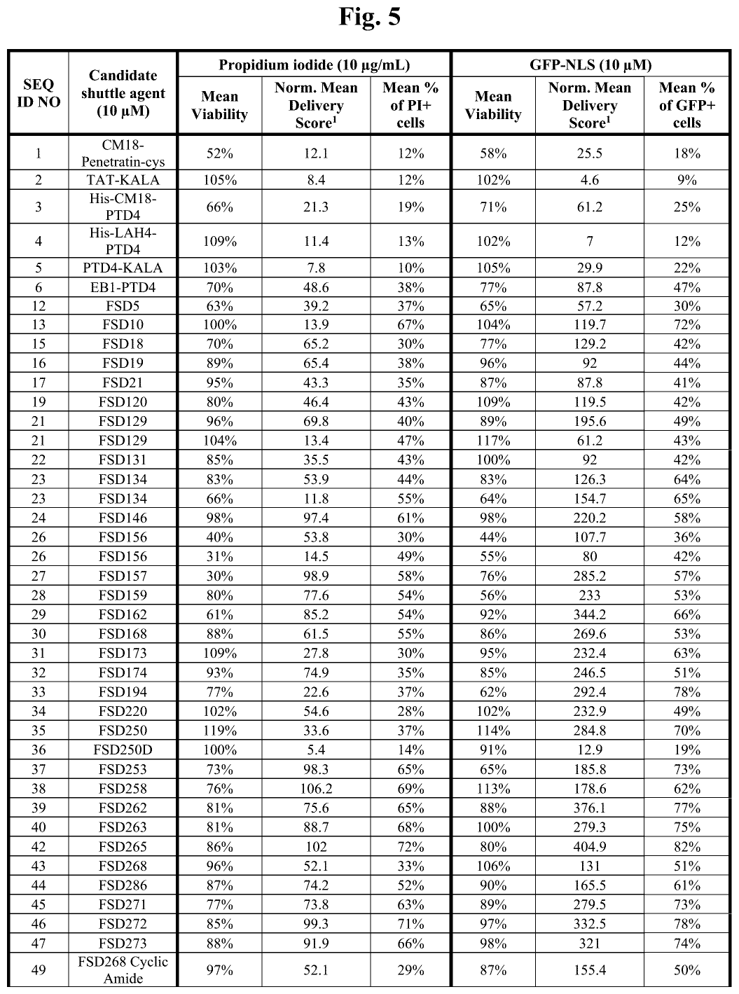

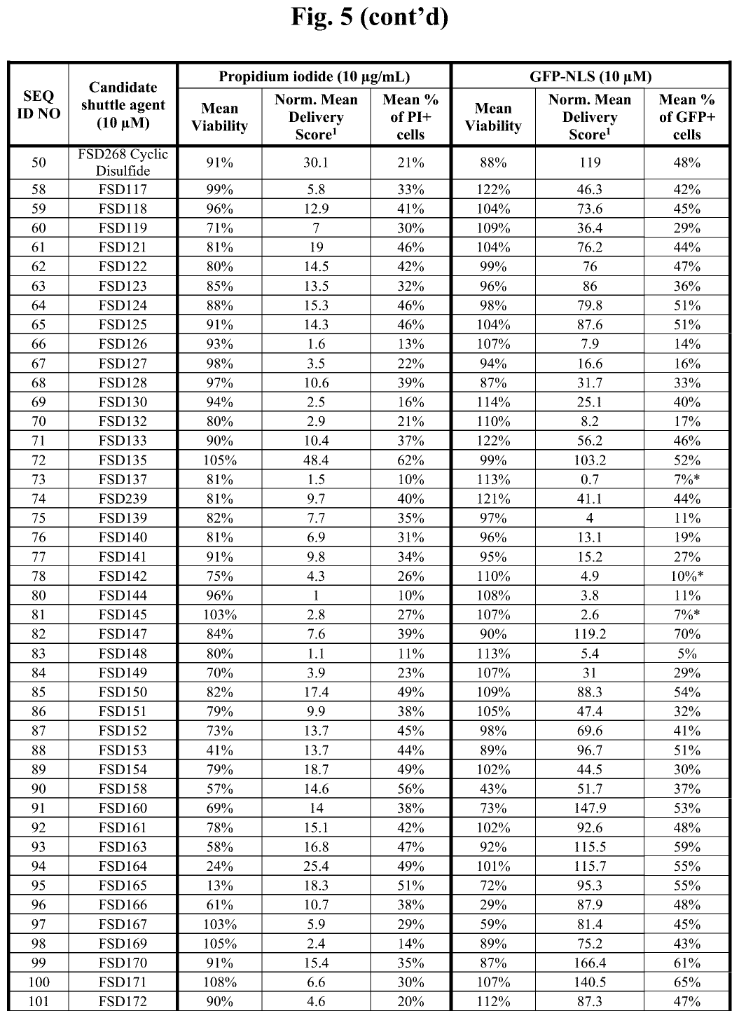

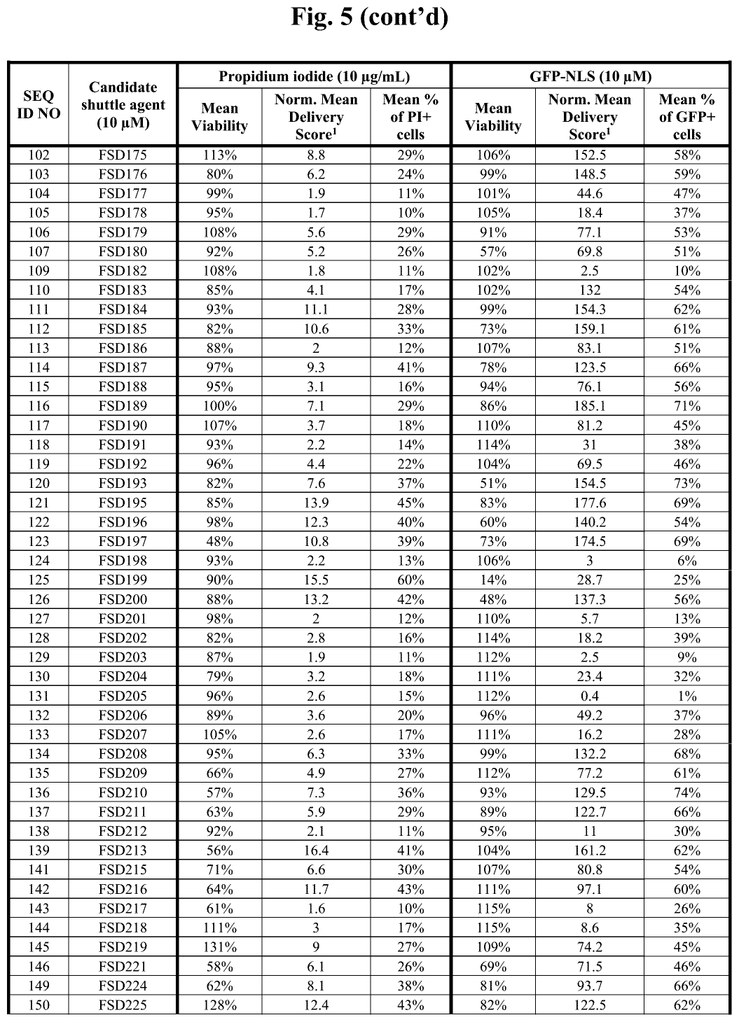

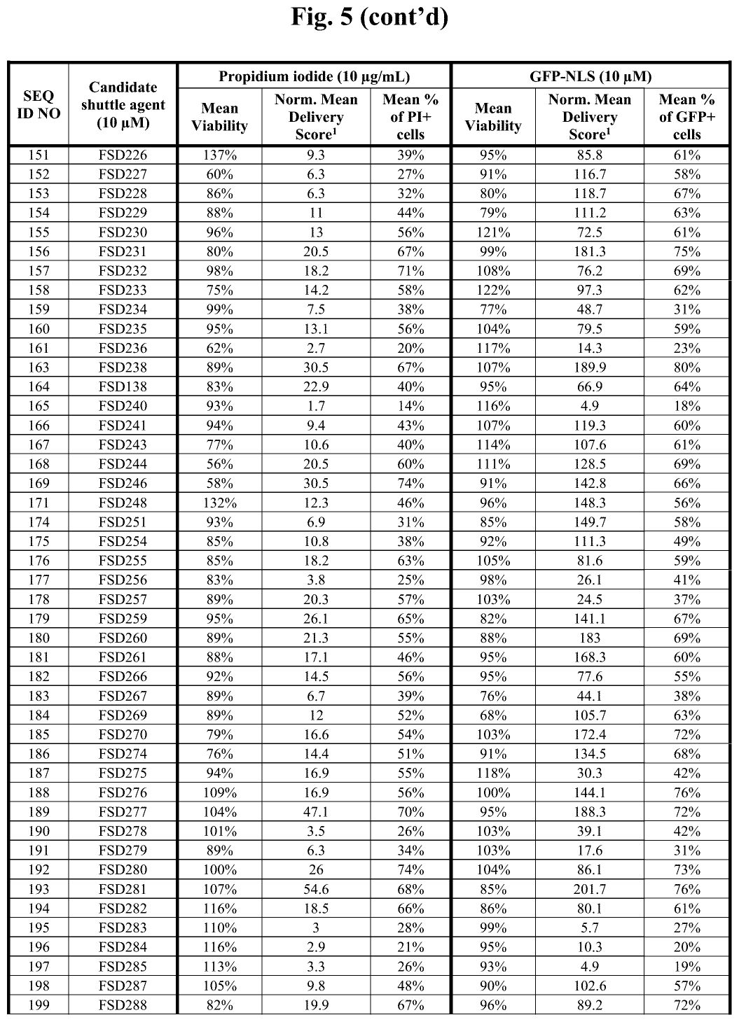

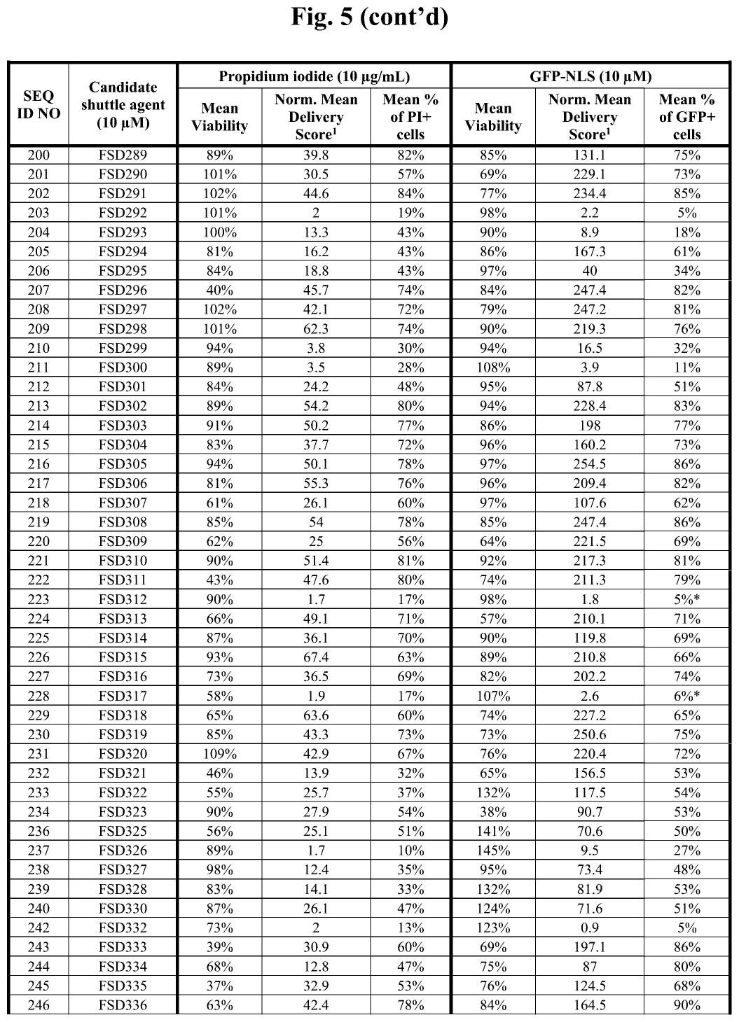

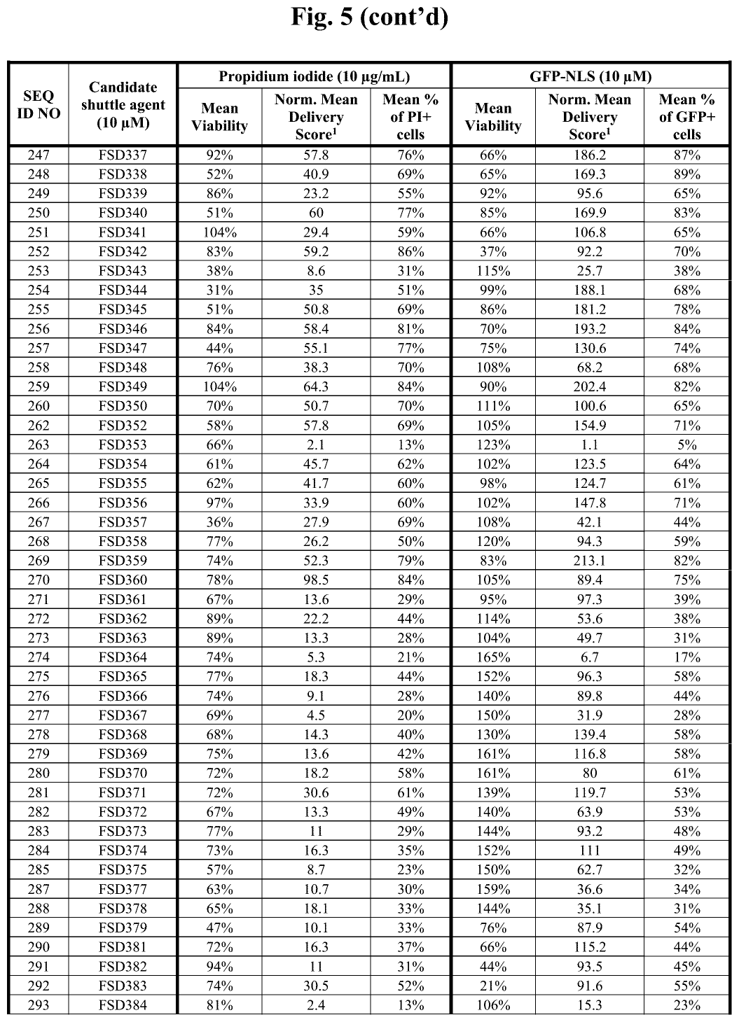

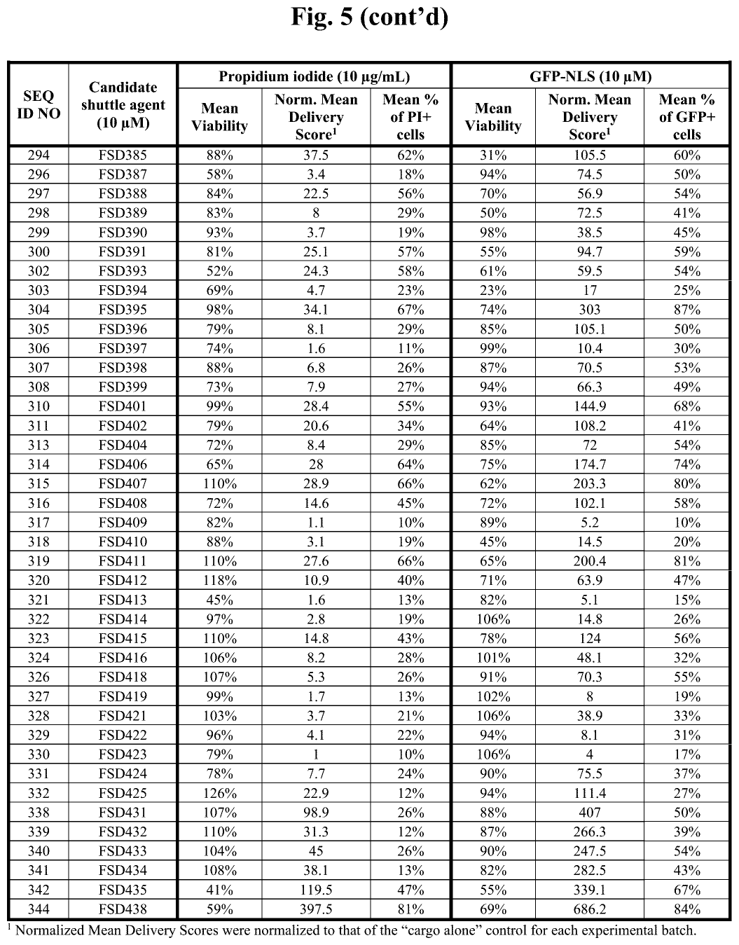

In some embodiments, shuttle agents described herein for delivery of non-anionic polynucleotide analog cargoes are preferably second generation shuttle agents lacking a cell-penetrating domain or lack a cell-penetrating domain fused to an endosome leakage domain. In some embodiments, shuttle agents described herein particularly suitable for delivery of non-anionic polynucleotide analog cargoes are preferably those having relatively high delivery scores, meaning that the shuttle agents deliver a greater total number of cargo molecules per cell. Since synthetic polynucleotide analogs described herein function by steric hindrance upon hybridizing to their target intracellular RNA molecules (i.e., one cargo molecule binds to one intracellular RNA molecule), it is expected that shuttle agents having higher delivery scores are particular advantageous for such applications. In some embodiments, shuttle agents described herein (and/or the SEQ ID NOs recited above in the preceding paragraph) are those listed in FIG. 5 having a Normalized Mean Delivery Score for delivery of PI and/or GFP cargoes of at least 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, or 200.

In some embodiments, the shuttle agents described herein comprise or consist of a variant of the synthetic peptide shuttle agent, the variant being identical to the synthetic peptide shuttle agent as defined herein, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said non-anionic polynucleotide analog cargo in eukaryotic cells as compared to in the absence of the synthetic peptide shuttle agent.

### Chemical Modifications and Synthetic Amino Acids

In some embodiments, shuttle agents of the present description may comprise oligomers (e.g., dimers, trimers, etc.) of peptides described herein. Such oligomers may be constructed by covalently binding the same or different types of shuttle agent monomers (e.g., using disulfide bridges to link cysteine residues introduced into the monomer sequences). In some embodiments, shuttle agents of the present description may comprise an N-terminal and/or a C-terminal cysteine residue.

In some embodiments, shuttle agents of the present description may comprise or consist of a cyclic peptide. In some embodiments, the cyclic peptide may be formed via a covalent link between a first residue positioned towards the N terminus of the shuttle agent and a second residue positioned towards the C terminus of the shuttle agent. In some embodiments, the first and second residues are flanking residues positioned at the N and the C termini of the shuttle agent. In some embodiments, the first and second residues may be linked via an amide linkage to form the cyclic peptide. In some embodiments, the cyclic peptide may be formed by a disulfide bond between two cysteine residues within the shuttle agent, wherein the two cysteine residues are positioned towards the N and C termini of the shuttle agent. In some embodiments, the shuttle agent may comprise, or be engineered to comprise, flanking cysteine residues at the N and C termini, which are linked via a disulfide bond to form the cyclic peptide. In some embodiments, the cyclic shuttle agents described herein may be more resistant to degradation (e.g., by proteases) and/or may have a longer half-life than a corresponding linear peptide.

In some embodiments, the shuttle agents of the present description may comprise one or more D-amino acids. In some embodiments, the shuttle agents of the present description may comprise a D-amino acid at the N and/or C terminus of the shuttle agent. In some embodiments, the shuttle agents maybe comprised entirely of D-amino acids. In some embodiments, the shuttle agents described herein having one or more D-amino acids may be more resistant to degradation (e.g., by proteases) and/or may have a longer half-life than a corresponding peptide comprised of only L-amino acids.

In some embodiments, the shuttle agents of the present description may comprise a chemical modification to one or more amino acids, wherein the chemical modification does not destroy the transduction activity of the synthetic peptide shuttle agent. As used herein in this context, the term “destroy” means that the chemical modification irreversibly abolishes the cargo transduction activity of a peptide shuttle agent described herein. Chemical modifications that may transiently inhibit, attenuate, or delay the cargo transduction activity of a peptide shuttle agent described herein may be included in the chemical modifications to the shuttle agents of the present description. In some embodiments, the chemical modification to any one of the shuttle agents described herein may be at the N and/or C terminus of the shuttle agent. Examples of chemical modifications include the addition of an acetyl group (e.g., an N-terminal acetyl group), a cysteamide group (e.g., a C-terminal cysteamide group), or a fatty acid (e.g., C4-C16, C6-C14, C6-C12, C6-C8, or C8 fatty acid, preferably being N-terminal).

In some embodiments, the shuttle agents of the present description comprise shuttle agent variants having transduction activity for non-anionic polynucleotide analog cargoes in target eukaryotic cells, the variants being identical to any shuttle agent of the present description, except having at least one amino acid being replaced with a corresponding synthetic amino acid or amino acid analog having a side chain of similar physiochemical properties (e.g., structure, hydrophobicity, or charge) as the amino acid being replaced. In some embodiments, the synthetic amino acid replacement:
(a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, 6-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, β-cyclopropylalanine, azetidine-2-carboxylic acid, α-allylglycine, propargylglycine, tert-butylalanine , β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β,β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, β-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, αaminoadipic acid, 2-aminoheptanedioic acid, α-aminosuberic acid, 4-carboxyphenylalanine, cysteic acid, 4-phosphonophenylalanine, or 4-sulfomethylphenylalanine

### Histidine-Rich Domains

In some embodiments, peptide shuttle agents of the present description may further comprise one or more histidine-rich domains. In some embodiments, the histidine-rich domain may be a stretch of at least 2, at least 3, at least 4, at least 5, or at least 6 amino acids comprising at least 30%, at least 35%, at least 40%, at least 45%, at least 50%, at least 55%, at least 60%, at least 65%, at least 70%, at least 75%, at least 80%, at least 85%, or at least 90% histidine residues. In some embodiments, the histidine-rich domain may comprise at least 2, at least 3, at least 4 at least 5, at least 6, at least 7, at least 8, or at least 9 consecutive histidine residues. Without being bound by theory, the histidine-rich domain in the shuttle agent may act as a proton sponge in the endosome through protonation of their imidazole groups under acidic conditions of the endosomes, providing another mechanism of endosomal membrane destabilization and thus further facilitating the ability of endosomally-trapped cargoes to gain access to the cytosol. In some embodiments, the histidine-rich domain may be located at or towards the N and/or C terminus of the peptide shuttle agent.

### Linkers

In some embodiments, peptide shuttle agents of the present description may comprise one or more suitable linkers (e.g., flexible polypeptide linkers). In some embodiments, such linkers may separate two or more amphipathic alpha-helical motifs (e.g., see the shuttle agent FSD18 in FIG. 49D of WO/2018/068135). In some embodiments, linkers can be used to separate two more domains (CPDs, ELDs, or histidine-rich domains) from one another. In some embodiments, linkers may be formed by adding sequences of small hydrophobic amino acids without rotatory potential (such as glycine) and polar serine residues that confer stability and flexibility. Linkers may be soft and allow the domains of the shuttle agents to move. In some embodiments, prolines may be avoided since they can add significant conformational rigidity. In some embodiments, the linkers may be serine/glycine-rich linkers (e.g., GS, GGS, GGSGGGS, GGSGGGSGGGS, or the like). In some embodiments, the use shuttle agents comprising a suitable linker may be advantageous for delivering a cargo to suspension cells, rather than to adherent cells. In some embodiments, the linker may comprise or consist of: -Gn-; -Sn-; -(GnSn)n-; -(GnSn)nGn-; -(GnSn)nSn-; -(GnSn)nGn(GnSn)n-; or -(GnSn)nSn(GnSn)n-, wherein G is the amino acid Gly; S is the amino acid Ser; and n is an integer from 1 to 5. In some embodiments, short stretches or “linkers” of flexible and/or hydrophilic amino acids (e.g., glycine/serine-rich stretches) may be added to the N terminus, C terminus, or both the N and C termini of a shuttle agent described herein, or a C-terminal truncated shuttle agent described herein. In some embodiments, such stretches may facilitate dissolution of shuttle agents, particularly shorter shuttle agents (e.g., having an amphipathic alpha helical structure with a strongly hydrophobic portion) that would otherwise be insoluble or only partially soluble in aqueous solution. In some embodiments, increasing the solubility of shuttle agent peptides may avoid the use of organic solvents (e.g., DMSO) that may obscure cargo transduction results and/or make the shuttle agents incompatible for therapeutic applications.

### Domain-Based Peptide Shuttle Agents

In some aspects, the shuttle agents described herein may be a shuttle agent as described in WO/2016/161516, comprising an endosome leakage domain (ELD) operably linked to a cell penetrating domain (CPD).

### Endosome Leakage Domains (ELDs)

In some aspects, peptide shuttle agents of the present description may comprise an endosome leakage domain (ELD) for facilitating endosome escape and access to the cytoplasmic compartment. As used herein, the expression “endosome leakage domain” refers to a sequence of amino acids which confers the ability of endosomally-trapped cargoes to gain access to the cytoplasmic compartment. Without being bound by theory, endosome leakage domains are short sequences (often derived from viral or bacterial peptides), which are believed to induce destabilization of the endosomal membrane and liberation of the endosome contents into the cytoplasm. As used herein, the expression “endosomolytic peptide” is intended to refer to this general class of peptides having endosomal membrane-destabilizing properties. Accordingly, in some embodiments, synthetic peptide or polypeptide-based shuttle agents of the present description may comprise an ELD which is an endosomolytic peptide. The activity of such peptides may be assessed for example using the calcein endosome escape assays described in Example 2 of WO/2016/161516.

In some embodiments, the ELD may be a peptide that disrupts membranes at acidic pH, such as pH-dependent membrane active peptide (PMAP) or a pH-dependent lytic peptide. For example, the peptides GALA and INF-7 are amphiphilic peptides that form alpha helixes when a drop in pH modifies the charge of the amino acids which they contain. More particularly, without being bound by theory, it is suggested that ELDs such as GALA induce endosomal leakage by forming pores and flip-flop of membrane lipids following conformational change due to a decrease in pH (Kakudo, Chaki et al., 2004, Li, Nicol et al., 2004). In contrast, it is suggested that ELDs such as INF-7 induce endosomal leakage by accumulating in and destabilizing the endosomal membrane (El-Sayed, Futaki et al., 2009). Accordingly, in the course of endosome maturation, the concomitant decline in pH causes a change in the conformation of the peptide and this destabilizes the endosome membrane leading to the liberation of the endosome contents. The same principle is thought to apply to the toxin A of Pseudomonas (Varkouhi, Scholte et al., 2011). Following a decline in pH, the conformation of the domain of translocation of the toxin changes, allowing its insertion into the endosome membrane where it forms pores (London 1992, O'Keefe 1992). This eventually favors endosome destabilization and translocation of the complex outside of the endosome. The above described ELDs are encompassed within the ELDs of the present description, as well as other mechanisms of endosome leakage whose mechanisms of action may be less well defined.

In some embodiments, the ELD may be an antimicrobial peptide (AMP) such as a linear cationic alpha-helical antimicrobial peptide (AMP). These peptides play a key role in the innate immune response due to their ability to strongly interact with bacterial membranes. Without being bound by theory, these peptides are thought to assume a disordered state in aqueous solution, but adopt an alpha-helical secondary structure in hydrophobic environments. The latter conformation thought to contribute to their typical concentration-dependent membrane-disrupting properties. When accumulated in endosomes at certain concentrations, some antimicrobial peptides may induce endosomal leakage.

In some embodiments, the ELD may be an antimicrobial peptide (AMP) such as Cecropin-A/Melittin hybrid (CM) peptide. Such peptides are thought to be among the smallest and most effective AMP-derived peptides with membrane-disrupting ability. Cecropins are a family of antimicrobial peptides with membrane-perturbing abilities against both Gram-positive and Gram-negative bacteria. Cecropin A (CA), the first identified antibacterial peptide, is composed of 37 amino acids with a linear structure. Melittin (M), a peptide of 26 amino acids, is a cell membrane lytic factor found in bee venom. Cecropin-melittin hybrid peptides have been shown to produce short efficient antibiotic peptides without cytotoxicity for eukaryotic cells (i.e., non-hemolytic), a desirable property in any antibacterial agent. These chimeric peptides were constructed from various combinations of the hydrophilic N-terminal domain of Cecropin A with the hydrophobic N-terminal domain of Melittin, and have been tested on bacterial model systems. Two 26-mers, CA(1-13)M(1-13) and CA(1-8) M(1-18) (Boman et al., 1989), have been shown to demonstrate a wider spectrum and improved potency of natural Cecropin A without the cytotoxic effects of melittin.

In an effort to produce shorter CM series peptides, the authors of Andreu et al., 1992 constructed hybrid peptides such as the 26-mer (CA(1-8)M(1-18)), and compared them with a 20-mer (CA(1-8)M(1-12)), a 18-mer (CA(1-8)M(1-10)) and six 15-mers ((CA(1-7)M(1-8), CA(1-7)M(2-9), CA(1-7)M(3-10), CA(1-7)M(4-11), CA(1-7)M(5-12), and CA(1-7)M(6-13)). The 20 and 18-mers maintained similar activity comparatively to CA(1-8)M(1-18). Among the six 15-mers, CA(1-7)M(1-8) showed low antibacterial activity, but the other five showed similar antibiotic potency compared to the 26-mer without hemolytic effect. Accordingly, in some embodiments, synthetic peptide or polypeptide-based shuttle agents of the present description may comprise an ELD which is or is from CM series peptide variants, such as those described above.

In some embodiments, the ELD may be the CM series peptide CM18 composed of residues 1-7 of Cecropin-A (KWKLFKKIGAVLKVLTTG) fused to residues 2-12 of Melittin (YGRKKRRQRRR), [C(1-7)M(2-12)]. When fused to the cell penetrating peptide TAT, CM18 was shown to independently cross the plasma membrane and destabilize the endosomal membrane, allowing some endosomally-trapped cargoes to be released to the cytosol (Salomone et al., 2012). However, the use of a CM18-TAT11 peptide fused to a fluorophore (atto-633) in some of the authors' experiments, raises uncertainty as to the contribution of the peptide versus the fluorophore, as the use of fluorophores themselves have been shown to contribute to endosomolysis—e.g., via photochemical disruption of the endosomal membrane (Erazo-Oliveras et al., 2014).

In some embodiments, the ELD may be CM18 having the amino acid sequence of SEQ ID NO: 1 of WO/2016/161516, ora variant thereof having at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identity to SEQ ID NO: 1 of WO/2016/161516 and having endosomolytic activity.

In some embodiments, the ELD may be a peptide derived from the N terminus of the HA2 subunit of influenza hemagglutinin (HA), which may also cause endosomal membrane destabilization when accumulated in the endosome.

In some embodiments, synthetic peptide or polypeptide-based shuttle agents of the present description may comprise an ELD which is or is from an ELD set forth in Table I, or a variant thereof having endosome escape activity and/or pH-dependent membrane disrupting activity.

TABLE I
Examples of endosome leakage domains

| | SEQ ID NO of | |
|---|---|---|
| Name | WO/2016/161516 | Reference(s) |
| CM18 | 1 | Salomone, Cardarelli et al., 2012 |
| Diphtheria toxin | 2 | Uherek, Fominaya et al., 1998, |
| T domain (DT) | | Glover, Ng et al., 2009 |
| GALA | 3 | Parente, Nir et al., 1990 Li, |
| | | Nicol et al., 2004 |
| PEA | 4 | Fominaya and Wels 1996 |
| INF-7 | 5 | El-Sayed, Futaki et al., 2009 |
| LAH4 | 6 | Kichler, Mason et al., 2006 |
| | | Kichler et al., 2003 |
| HGP | 7 | Kwon et al., 2010 |
| H5WYG | 8 | Midoux, Kichler et al., 1998 |
| HA2 | 9 | Lorieau, Louis et al., 2010 |
| EB1 | 10 | Amand, Norden et al., 2012 |
| VSVG | 11 | Schuster, Wu et al., 1999 |
| Pseudomonas toxin | 12 | Fominaya, Uherek et al., 1998 |
| Melittin | 13 | Tan, Chen et al., 2012 |
| KALA | 14 | Wyman, Nicol et al., 1997 |
| JST-1 | 15 | Gottschalk, Sparrow et al., 1996 |
| C(LLKK)₃C | 63 | Luan et al., 2015 |
| G(LLKK)₃G | 64 | Luan et al., 2015 |

In some embodiments, shuttle agents of the present description may comprise one or more ELD or type of ELD. More particularly, they can comprise at least 2, at least 3, at least 4, at least 5, or more ELDs. In some embodiments, the shuttle agents can comprise between 1 and 10 ELDs, between 1 and 9 ELDs, between 1 and 8 ELDs, between 1 and 7 ELDs, between 1 and 6 ELDs, between 1 and 5 ELDs, between 1 and 4 ELDs, between 1 and 3 ELDs, etc.

In some embodiments, the order or placement of the ELD relative to the other domains (CPD, histidine-rich domains) within the shuttle agents of the present description may be varied provided the shuttling ability of the shuttle agent is retained.

In some embodiments, the ELD may be a variant or fragment of any one those listed in Table I, and having endosomolytic activity. In some embodiments, the ELD may comprise or consist of the amino acid sequence of any one of SEQ ID NOs: 1-15, 63, or 64 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 1-15, 63, or 64 of WO/2016/161516, and having endosomolytic activity.

In some embodiments, shuttle agents of the present description do not comprise one or more of the amino acid sequences of any one of SEQ ID NOs: 1-15, 63, or 64 of WO/2016/161516.

### Cell Penetration Domains (CPDs)

In some aspects, the shuttle agents of the present description may comprise a cell penetration domain (CPD). As used herein, the expression “cell penetration domain” refers to a sequence of amino acids which confers the ability of a macromolecule (e.g., peptide or protein) containing the CPD to be transduced into a cell.

In some embodiments, the CPD may be (or may be from) a cell-penetrating peptide or the protein transduction domain of a cell-penetrating peptide. Cell-penetrating peptides can serve as carriers to successfully deliver a variety of cargoes intracellularly (e.g., polynucleotides, polypeptides, small molecule compounds or other macromolecules/compounds that are otherwise membrane-impermeable). Cell-penetrating peptides often include short peptides rich in basic amino acids that, once fused (or otherwise operably linked) to a macromolecule, mediate its internalization inside cells (Shaw, Catchpole et al., 2008). The first cell-penetrating peptide was identified by analyzing the cell penetration ability of the HIV-1 trans-activator of transcription (Tat) protein (Green and Loewenstein 1988, Vives, Brodin et al., 1997). This protein contains a short hydrophilic amino acid sequence, named “TAT”, which promotes its insertion within the plasma membrane and the formation of pores. Since this discovery, many other cell-penetrating peptides have been described. In this regard, in some embodiments, the CPD can be a cell-penetrating peptide as listed in Table II, or a variant thereof having cell-penetrating activity.

TABLE II
Examples of cell-penetrating peptides

| | SEQ ID NO of | |
|---|---|---|
| Name | WO/2016/161516 | Reference(s) |
| SP | 16 | Mahlum, Mandal et al., 2007 |
| TAT | 17 | Green and Loewenstein 1988, |
| | | Fawell, Seery et al., 1994, |
| | | Vives, Brodin et al., 1997 |
| Penetratin | 18 | Perez, Joliot et al., 1992 |
| (Antennapedia) | | |
| pVEC | 19 | Elmquist, Lindgren et al., 2001 |
| M918 | 20 | El-Andaloussi, Johansson et al., 2007 |
| Pep-1 | 21 | Morris, Depollier et al., 2001 |
| Pep-2 | 22 | Morris, Chaloin et al., 2004 |
| Xentry | 23 | Montrose, Yang et al., 2013 |
| Arginine | 24 | Zhou, Wu et al., 2009 |
| stretch | | |
| Transportan | 25 | Hallbrink, Floren et al., 2001 |
| SynB1 | 26 | Drin, Cottin et al., 2003 |
| SynB3 | 27 | Drin, Cottin et al., 2003 |
| PTD4 | 65 | Ho et al, 2001 |

Without being bound by theory, cell-penetrating peptides are thought to interact with the cell plasma membrane before crossing by pinocytosis or endocytosis. In the case of the TAT peptide, its hydrophilic nature and charge are thought to promote its insertion within the plasma membrane and the formation of a pore (Herce and Garcia 2007). Alpha helix motifs within hydrophobic peptides (such as SP) are also thought to form pores within plasma membranes (Veach, Liu et al., 2004).

In some embodiments, shuttle agents of the present description may comprise one or more CPD or type of CPD. More particularly, they may comprise at least 2, at least 3, at least 4, or at least 5 or more CPDs. In some embodiments, the shuttle agents can comprise between 1 and 10 CPDs, between 1 and 6 CPDs, between 1 and 5 CPDs, between 1 and 4 CPDs, between 1 and 3 CPDs, etc.

In some embodiments, the CPD may be TAT having the amino acid sequence of SEQ ID NO: 17 of WO/2016/161516., ora variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 17 of WO/2016/161516 and having cell penetrating activity; or Penetratin having the amino acid sequence of SEQ ID NO: 18 of WO/2016/1615 16, or a variant thereof having at least 70%, 71%, 72%, 73%, 74%, 75%, 76%, 77%, 78%, 79%, 80%, 81% 82%, 83%, 84%, 85%, 86%, 87%, 88%, 89%, 85%, 86%, 87%, 88%, 89%, 90%, 91%, 92%, 93%, 94%, or 95% identity to SEQ ID NO: 18 of WO/2016/161516 and having cell penetrating activity.

In some embodiments, the CPD may be PTD4 having the amino acid sequence of SEQ ID NO: 65 of WO/2016/161516, ora variant thereof having at least 70%, 71%, 72%, 73%, 74%, 75%, 76%, 77%, 78%, 79%, 80%, 81% 82%, 83%, 84%, 85%, 86%, 87%, 88%, 89%, 90%, 91%, 92%, 93%, 94%, or 95% identity to SEQ ID NO: 65 of WO/2016/161516.

In some embodiments, the order or placement of the CPD relative to the other domains (ELD, histidine-rich domains) within the shuttle agents of the present description may be varied provided the transduction ability of the shuttle agent is retained.

In some embodiments, the CPD may be a variant or fragment of any one those listed in Table II, and having cell penetrating activity. In some embodiments, the CPD may comprise or consist of the amino acid sequence of any one of SEQ ID NOs: 16-27 or 65 of WO/2016/161516, or a sequence which is at least 70%, 71%, 72%, 73%, 74%, 75%, 76%, 77%, 78%, 79%, 80%, 81%, 82%, 83%, 84%, 85%, 86%, 87%, 88%, 89%, 85%, 90%, 91%, 92%, 93%, 94%, or 95% identical to any one of SEQ ID NOs: 16-27 or 65 of WO/2016/161516., and having cell penetrating activity.

In some embodiments, shuttle agents of the present description do not comprise any one of the amino acid sequences of SEQ ID NOs: 16-27 or 65 of WO/2016/161516.

### Methods, Kits, Uses, Compositions, and Cells

In some embodiments, the present description relates to methods for delivering a non-anionic polynucleotide analog cargo from an extracellular space to the cytosol and/or nucleus of a target eukaryotic cell. The methods comprise contacting the target eukaryotic cell with the cargo in the presence of a shuttle agent at a concentration sufficient to increase the transduction efficiency of said cargo, as compared to in the absence of said shuttle agent. In some embodiments, contacting the target eukaryotic cell with the cargo in the presence of the shuttle agent results in an increase in the transduction efficiency of said non-anionic polynucleotide analog cargo by at least 10-fold, 20-fold, 30-fold, 40-fold, 50-fold, or 100-fold, as compared to in the absence of said shuttle agent.

In some embodiments, the present description relates to a method for increasing the transduction efficiency of a non-anionic polynucleotide analog cargo to the cytosol and/or nucleus of target eukaryotic cells. As used herein, the expression “increasing transduction efficiency” refers to the ability of a shuttle agent of the present description to improve the percentage or proportion of a population of target cells into which a cargo of interest (e.g., non-anionic polynucleotide analog cargo) is delivered intracellularly. Immunofluorescence microscopy, flow cytometry, and other suitable methods may be used to assess cargo transduction efficiency. In some embodiments, a shuttle agent of the present description may enable a transduction efficiency of at least 25%, 30%, 35%, 40%, 45%, 50%, 55%, 60%, 65%, 70%, 75%, 80%, or 85%, for example as measured by immunofluorescence microscopy, flow cytometry, FACS, and other suitable methods. In some embodiments, a shuttle agent of the present description may enable one of the aforementioned transduction efficiencies together wish a cell viability of at least 25%, 30%, 35%, 40%, 45%, 50%, 55%, 60%, 65%, 70%, 75%, 80%, 85%, 90%, or 95%, for example as measured by the assay described in Example 3.3a of WO/2018/068135, or by another suitable assay known in the art.

In addition to increasing target cell transduction efficiency, shuttle agents of the present description may facilitate the delivery of a cargo of interest (e.g., non-anionic polynucleotide analog cargo) to the cytosol and/or nucleus of target cells. In this regard, efficiently delivering an extracellular cargo to the cytosol and/or nucleus of a target cell using peptides can be challenging, as the cargo often becomes trapped in intracellular endosomes after crossing the plasma membrane, which may limit its intracellular availability and may result in its eventual metabolic degradation. For example, use of the protein transduction domain from the HIV-1 Tat protein has been reported to result in massive sequestration of the cargo into intracellular vesicles. In some aspects, shuttle agents of the present description may facilitate the ability of endosomally-trapped cargo to escape from the endosome and gain access to the cytoplasmic compartment. In this regard, the expression “to the cytosol” for example in the phrase “increasing the transduction efficiency of a non-anionic polynucleotide analog cargo to the cytosol,” is intended to refer to the ability of shuttle agents of the present description to allow an intracellularly delivered cargo of interest to escape endosomal entrapment and gain access to the cytoplasmic and/or nuclear compartment. After a cargo of interest has gained access to the cytosol, it may be free to bind to its intracellular target (e.g., in the cytosol, nucleus, nucleolus, mitochondria, peroxisome). In some embodiments, the expression “to the cytosol” is thus intended to encompass not only cytosolic delivery, but also delivery to other subcellular compartments that first require the cargo to gain access to the cytoplasmic compartment.

In some embodiments, the methods of the present description are in vitro methods (e.g., such as for therapeutic and/or diagnostic purpose). In other embodiments, the methods of the present description are in vivo methods (e.g., such as for therapeutic and/or diagnostic purpose). In some embodiments, the methods of the present description comprise topical, enteral/gastrointestinal (e.g., oral), or parenteral administration of the non-anionic polynucleotide analog cargo and the synthetic peptide shuttle agent. In some embodiments, described herein are compositions formulated for topical, enteral/gastrointestinal (e.g., oral), or parenteral administration of the non-anionic polynucleotide analog cargo and the synthetic peptide shuttle agent.

In some embodiments, the methods of the present description may comprise contacting the target eukaryotic cell with the shuttle agent, or composition as defined herein, and the non-anionic polynucleotide analog cargo. In some embodiments, the shuttle agent, or composition may be pre-incubated with the cargo to form a mixture, prior to exposing the target eukaryotic cell to that mixture. In some embodiments, the type of shuttle agent may be selected based on the identity and/or physicochemical properties of the cargo to be delivered intracellularly. In other embodiments, the type of shuttle agent may be selected to take into account the identity and/or physicochemical properties of the cargo to be delivered intracellularly, the type of cell, the type of tissue, etc.

In some embodiments, the method may comprise multiple treatments of the target cells with the shuttle agent, or composition (e.g., 1, 2, 3, 4 or more times per day, and/or on a pre-determined schedule). In such cases, lower concentrations of the shuttle agent, or composition may be advisable (e.g., for reduced toxicity). In some embodiments, the cells may be suspension cells or adherent cells. In some embodiments, the person of skill in the art will be able to adapt the teachings of the present description using different combinations of shuttles, domains, uses and methods to suit particular needs of delivering a non-anionic polynucleotide analog cargo to particular cells with a desired viability.

In some embodiments, the methods of the present description may apply to methods of delivering a non-anionic polynucleotide analog cargo intracellularly to a cell in vivo. Such methods may be accomplished by parenteral administration or direct injection into a tissue, organ, or system.

In some aspects, the compositions or synthetic peptide shuttle agents of the present description may be for use in an in vitro or in vivo method for increasing the transduction efficiency of a non-anionic polynucleotide analog cargo (e.g., targeting a therapeutically or biologically relevant RNA molecule) into target eukaryotic cells, wherein the synthetic peptide shuttle agent or synthetic peptide shuttle agent variant is used or is formulated for use at a concentration sufficient to increase the transduction efficiency and cytosolic and/or nuclear delivery of the cargo into the target eukaryotic cells, as compared to in the absence of the synthetic peptide shuttle agent or synthetic peptide shuttle agent variant.

In some embodiments, compositions or synthetic peptide shuttle agents of the present description may be for use in therapy, wherein the synthetic peptide shuttle agent or synthetic peptide shuttle agent variant transduces a therapeutically relevant non-anionic polynucleotide analog cargo to the cytosol and/or nucleus of target eukaryotic cells, wherein the synthetic peptide shuttle agent or synthetic peptide shuttle agent variant is used (or is formulated for use) at a concentration sufficient to increase the transduction efficiency of the cargo into the target eukaryotic cells, as compared to in the absence of the synthetic peptide shuttle agent.

In some aspects, described herein is a composition for use in transducing a non-anionic polynucleotide analog cargo into target eukaryotic cells, the composition comprising a synthetic peptide shuttle agent formulated with a pharmaceutically suitable excipient, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent. In some embodiments, the composition further comprises the cargo. In some embodiments, the composition may be mixed with the cargo prior to administration or therapeutic use.

In some aspects, described herein is a composition for use in therapy, the composition comprising a synthetic peptide shuttle agent formulated with a non-anionic polynucleotide analog cargo to be transduced into target eukaryotic cells by the synthetic peptide shuttle agent, wherein the concentration of the synthetic peptide shuttle agent in the composition is sufficient to increase the transduction efficiency and cytosolic and/or nuclear delivery of the cargo into said target eukaryotic cells upon administration, as compared to in the absence of said synthetic peptide shuttle agent.

In some aspects, described herein is a composition comprising a non-anionic polynucleotide analog cargo for intracellular delivery and a synthetic peptide shuttle agent that is independent from, or is not covalently linked to, said non-anionic polynucleotide analog cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said non-anionic polynucleotide analog cargo in eukaryotic cells as compared to in the absence of the synthetic peptide shuttle agent. In some embodiments, the compositions and/or shuttle agents described herein do not comprise an organic solvent (e.g., DMSO), or do not comprise a concentration of an organic solvent not suitable for therapeutic or human use. In some embodiments, the shuttle agents described herein are advantageously designed with aqueous solubility in mind, thereby precluding the necessity of using organic solvents.

In some embodiments, the shuttle agent, or composition, and the non-anionic polynucleotide analog cargo may be exposed to the target cell in the presence or absence of serum. In some embodiments, the method may be suitable for clinical or therapeutic use.

In some embodiments, the present description relates to a kit for delivering a non-anionic polynucleotide analog cargo from an extracellular space to the cytosol and/or nucleus of a target eukaryotic cell. In some embodiments, the present description relates to a kit for increasing the transduction efficiency of a non-anionic polynucleotide analog cargo to the cytosol of a target eukaryotic cell. The kit may comprise the shuttle agent, or composition as defined herein, and a suitable container.

In some embodiments, the target eukaryotic cells may be an animal cell, a mammalian cell, or a human cell. In some embodiments, the target eukaryotic cells may be stem cells (e.g., embryonic stem cells, pluripotent stem cells, induced pluripotent stem cells, neural stem cells, mesenchymal stem cells, hematopoietic stem cells, peripheral blood stem cells), primary cells (e.g., myoblast, fibroblast), immune cells (e.g., NK cell, T cell, dendritic cell, antigen presenting cell), epithelial cells, skin cells, gastrointestinal cells, mucosal cells, or pulmonary (lung) cells. In some embodiments, target cells comprise those having the cellular machinery for endocytosis (i.e., to produce endosomes).

In some embodiments, the present description relates to an isolated cell comprising a synthetic peptide shuttle agent as defined herein. In some embodiments, the cell may be a pluripotent stem cell. It will be understood that cells that are often resistant or not amenable to DNA transfection may be interesting candidates for the synthetic peptide shuttle agents of the present description.

In some embodiments, the present description relates to a composition or method described herein, wherein the non-anionic polynucleotide analog cargo is a non-anionic antisense oligonucleotide targeting a gene of the Hedgehog pathway. In some embodiments, the non-anionic antisense oligonucleotide targets Gli1 for knockdown. In some embodiments, the non-anionic antisense oligonucleotide hybridizes (e.g., when in the cytosol or under cytosolic conditions) to the polynucleotide sequence of any one of SEQ ID NOs: 365-368. In some embodiments, the non-anionic antisense oligonucleotide described herein comprises a sequence that hybridizes to any one of SEQ ID NOs: 365-368. In some embodiments, the present description relates to a composition or method described herein, wherein the composition or method of for the treatment of Gorlin's syndrome and/or basal cell carcinoma.

### EXAMPLES

### Example 1: Materials and Methods

All materials and methods not described or specified herein were generally as performed in WO/2018/068135, CA 3,040,645 or WO/2020/210916.

### Materials and Reagents

| Material | Company | City, Province-State, Country |
|---|---|---|
| DMEM | Sigma-Aldrich | Oakville, ON, Canada |
| Fetal bovine serum (FBS) | NorthBio | Toronto, ON, Canada |
| L-glutamine-Penicillin-Streptomycin | Sigma-Aldrich | Oakville, ON, Canada |
| RPMI-1640 media | Sigma-Aldrich | Oakville, ON, Canada |
| Opti-MEM ™ | Sigma-Aldrich | Oakville, ON, Canada |
| PMO-Wnt1 (SEQ ID NO: 347) | Gene Tools, LLC | Philomath, OR, United States |
| PMO-Gli1 (SEQ ID NO: 346) | Gene Tools, LLC | Philomath, OR, United States |
| Fluoro Standard Control - PMO-FITC | Gene Tools, LLC | Philomath, OR, United States |
| (SEQ ID NO: 348) | (#PCO-GFPControl-100) | |
| PMO-GFP (SEQ ID NO: 345) | Gene Tools, LLC | Philomath, OR, United States |
| SiRNA-GFP (SEQ ID NO: 349) | Horizon Discovery/ | Lafayette, CO, USA |
| | Dharmacon | |
| siRNA-Gli1 (SEQ ID NO: 350) | Horizon Discovery/ | Lafayette, CO, USA |
| | Dharmacon | |
| Lipofectamine ™ RNAiMax | ThermoFisher # 13778100 | Burlington, ON, Canada |
| Pierce ™ BCA Protein Assay kit | ThermoFisher Scientific | Burlington, ON, Canada |
| | #23225 | |
| Alpha-Actinin (D6F6) XP ® Rabbit mAb | New England Biolab #6487S | Whitby, ON, Canada |
| Anti-Gli1 primary antibody | Abcam #ab273018 | Toronto, ON, Canada |
| HRP conjugated Goat anti-rabbit IgG | Abcam #6721 | Toronto, ON, Canada |
| polyclonal Secondary Antibody | | |
| Bovine Serum Albumin (BSA) | Bio Shop Canada Inc. | Burlington, ON, Canada |
| | #ALB007.100 | |

### Cell Lines and Culture Conditions

Cells were cultured following the manufacturer's instructions.

| | | | Culture | | |
|---|---|---|---|---|---|
| Cell lines | Description | ATCC/others | media | Serum | Additives |
| HeLa | Human cervical | ATCCTM CCL-2 | DMEM | 10% FBS | L-glutamine 2 mM |
| | carcinoma cells | | | | Penicillin 100 units |
| | | | | | Streptomycin 100 μg/mL |
| HeLa-plex | HeLa cells stably | | DMEM | 10% FBS | L-glutamine 2 mM |
| TetO-GFPd | expressing an | | | | Penicillin 100 units |
| | unstable GFP | | | | Streptomycin 100 μg/mL |
| | (“GFPd”) | | | | |
| DU145 | Human prostate | ATCCR HTB- | RPMI-1640 | 10% FBS | L-glutamine 2 mM |
| | carcinoma cells | 81TM | | | Penicillin 100 units |
| | | | | | Streptomycin 100 μg/mL |
| RH-30 | Human | ATCC CRL-2061 | RPMI-1640 | 10% FBS | L-glutamine 2 mM |
| | rhabdomyosarcoma | | | | Penicillin 100 units |
| | cell line | | | | Streptomycin 100 μg/mL |

### Phosphorodiamidate Morpholino Oligomer Transduction Protocol

Phosphorodiamidate morpholino oligomers labeled with the fluorophore FITC (PMO-FITC) were prepared at 1 mM in sterile water. HeLa cells were plated (20 000 cells/well) in a 96 well-dish the day prior to the experiment. Each delivery mix comprising a synthetic peptide shuttle agent (7.5, 10 or 20 μM) and a PMO-FITC (6 μM) was prepared and completed to 50 μL with RPMI-1640 media. Cells were washed once with PBS and the shuttle agent/PMO-FITC or PMO-FITC alone added on cells for five minutes. Then, 100 μL DMEM containing 10% FBS was added to the mix and removed. Cells were washed once with PBS and incubated in DMEM containing 10% FBS. Cells were analyzed after a 2-hour incubation by flow cytometry.

### Antisense GFP-PMO Transduction Protocol in GFPd Reporter HeLa Cells

Stock solutions of cargoes were prepared as follows: PMO stocks (1 mM in water); siRNA stocks (100 μM in 60 mM KCl, 6 mM HEPES-pH 7.5, and 0.2 mM MgCl₂).

Delivery. HeLa-plex-TetO-GFPd cells were plated (20 000 cells/well) in a 96 well-dish the day to prior the experiment. Each delivery mix comprising a synthetic peptide shuttle agent (7.5 μM) and a PMO (0.1 or 10 μM) was prepared and completed to 50 μL with RPMI-1640 media. Cells were washed once with PBS and the shuttle agent/PMO or PMO alone added on cells for five minutes. Then, 100 μL DMEM containing 10% FBS was added to the mix and removed. Cells were washed once with PBS and incubated in DMEM containing 10% FBS. Cells were analyzed after a 5-hour incubation by flow cytometry.

Transfection. HeLa-plex-TetO-GFPd cells were plated (20 000 cells/well) in a 96 well-dish the day prior to the experiment. siRNA (2.5 pmol) were transfected using the Lipofectamine™ RNAiMax reagent following the manufacturer's instructions. Lipofectamine™ RNAiMax was diluted in Opti-MEM (0.3 μL in 25 siRNA stocks were first diluted at 10 μM in RNAse free water then 2.5 pmol (0.25 μL) was added to 25 μL Opti-MEM. Diluted Lipofectamine™ RNAiMax was mixed with diluted siRNA (50 nM final concentration) and incubated 5 minutes at room temperature. Cells were washed once with PBS and 100 μL of DMEM containing 10% FBS were added on cells. The siRNA diluted in Lipofectamine™ RNAiMax was added to cells. After 24 h, media was changed for 100 μL of fresh DMEM containing 10% FBS. Cells were analyzed 48 hours post transfection by flow cytometry.

### Antisense PMO Transduction Protocol to Knock-Down Gli1 Expression in DU145 Cells

DU145 cells were trypsinized and plated (500 000 cells/well) in a 24 well-dish the day prior to the experiment. Each delivery mix comprising a synthetic peptide shuttle agent (5 μM) and PMO-FITC (6 μM) alone or with antisense PMOs (6 μM) designed to knock-down expression of targeted proteins were prepared and completed to 1 mL with plain RPMI-1640 medium. Cells were washed once with PBS and delivery mixes were added on cells for five minutes. Then, 2 mL of RPMI-1640 containing 10% FBS was added to the mix and removed. Untreated cells were incubated with RPMI-1640 only. Cells were incubated in fresh RPMI-1640 containing 10% FBS. Forty height hours later, medium was removed and cells were washed once with PBS prior to trypsinization. Cells were harvested, collected by centrifugation, washed, and resuspended with PBS. Then, PMO-FITC positive and PMO-FITC negative cells were sorted and collected by virtue of FACS (BD FACS Aria Fusion). FITC-positive and FITC-negative cell samples were collected by centrifugation and resuspended in 50 μL protein extraction RIPA buffer (150 mM NaCl, 1% Nonidet™ P-40, 0.1% SDS, 0.5% Sodium deoxycholate, 25 mM Tris). Total protein concentrations were measured with a BCA protein assay kit. For all conditions, 10 μg of proteins were prepared at a final volume of 40 μL with 4× Laemmeli and RIPA buffer. Protein samples were then heated at 90° C. and separated through an 8% SDS-PAGE gel. Proteins were then transferred over night (25 volts) onto a 0.2 μM PVDF membrane. Membranes were blocked using a 5% bovine serum albumin, Tris Buffer Saline, 0.1% Tween® 20 solution (5% BSA/TBS-T) for an hour. After blocking, the membrane was incubated for an hour with a 5% BSA/TBS-T solution containing anti alpha-Actinin (D6F6) primary antibody at a 1:1000. Alpha-Actinin protein present in the cell lysates served as loading control. Subsequently, the membranes were incubated over night with a 5% BSA/TBS-T solution containing anti-Gli1 primary antibody diluted 1:500. The membrane was then subjected to a 1-hour incubation at 1:5000 dilution of HRP-conjugated goat anti-rabbit secondary antibody 5% BSA/TBS-T solution. Chemiluminescence detection was performed using Clarity™ Western ECL Substrate and a ChemiDoc™ XRS apparatus. Gli1 and Alpha-Actinin densitometry was assessed using ImageJ™ software.

### Propidium Iodide or GFP-NLS Transduction Protocols

HeLa cells were plated (20 000 cells/well) in a 96 well-dish the day prior to the experiment. Each delivery mix comprising a synthetic peptide shuttle agent (10 μM) and the propidium iodide (PI) (10 μg/mL) or the GFP-NLS (10 μM) were prepared and completed to 50 μL with phosphate-buffered saline (PBS) for PI or with RPMI-1640 medium for GFP-NLS. Cells were washed once and the shuttle agent/PI or shuttle agent/GFP-NLS added on cells for one minute (PI) or 5 minutes (GFP-NLS). Then 100 μL DMEM containing 10% FBS was added to the mix and removed. Cells were washed once with PBS and incubated in DMEM containing 10% FBS. Cells were analyzed after 2-hour incubation by flow cytometry. Cells were analyzed one hour after PI or GFP-NLS treatment.

### Example 2: Synthetic Peptide Shuttle Agents: a New Class of Intracellular Delivery Peptides

Synthetic peptides called shuttle agents represent a new class of intracellular delivery peptides having the ability to rapidly transduce polypeptide cargoes to the cytosolic/nuclear compartment of eukaryotic cells. In contrast to traditional cell penetrating peptide-based intracellular delivery strategies, synthetic peptide shuttle agents are not covalently linked to their polypeptide cargoes. In fact, covalently linking shuttle agents to their cargoes in a non-cleavable manner generally has a negative effect on their transduction activity.

The first generation of synthetic peptide shuttle agents was described in WO/2016/161516 and consisted of multi-domain-based peptides having an endosome leakage domain (ELD) operably linked to a cell penetrating domain (CPD), and optionally further comprising one or more histidine-rich domains. Although it was initially believed that shuttle agent-mediated cargo transduction occurred via mechanisms similar to that of conventional cell-penetrating peptides, the speed and efficiency of cargo delivery to the cytosolic/nuclear compartment suggested a strong contribution from a more direct delivery mechanism across the plasma membrane without requiring complete endosomal formation (Del'Guidice et al., 2018). Therefore, using the first generation shuttle agents as a starting point, a large scale iterative design and screening program was undertaken to optimize the shuttle agents for the rapid and efficient transduction of polypeptide cargoes while reducing cellular toxicity. The program involved the manual and computer-assisted design/modeling of almost 11,000 synthetic peptides, as well as the synthesis and testing of several hundred different peptides for their ability to transduce a variety of polypeptide cargoes rapidly and efficiently in a plurality of cells and tissues. Rather than considering the shuttle agents as fusions of known cell-penetrating peptides (CPDs) and endosomolytic peptides (ELDs) derived from the literature, each peptide was considered holistically based on their predicted three-dimensional structure and physicochemical properties. The design and screening program culminated in a second generation of synthetic peptide shuttle agents defined by a set of fifteen parameters described in WO/2018/068135 governing the rational design of shuttle agents with improved transduction/toxicity profiles for polypeptide cargoes over the first generation shuttle agents. These second generation synthetic peptide shuttle agents were designed and empirically screened for the rapid transduction of polypeptide cargoes (i.e., typically within under 5 minutes) and thus were predominantly designed to lack a prototypical CPD.

### Example 3: Inefficient Delivery of Naked DNA/RNA Cargoes to the Cytosolic/Nuclear Compartment by Synthetic Peptide Shuttle Agents

Cell penetrating peptides (CPPs) have been used for decades in transfection strategies to deliver DNA/RNA intracellularly. The delivery of polynucleotides using CPPs can be divided into two categories in which the CPPs are either covalently bound or electrostatically bound to their polynucleotide cargo. The increased complexity in the synthesis of the former is a significant hurdle, while the latter is relatively simple given the cationic nature of CPPs and the negatively-charged phosphate backbone of DNA/RNA. Thus, during the screening of first generation synthetic peptide shuttle agents, experiments were performed to determine whether the shuttle agents could efficiently transduce plasmid DNA cargo to the nucleus for gene expression. Example 7.2 of WO/2016/161516 reported the results of these transfection experiments, in which the first generation shuttle agent CM18-TAT-Cys was indeed able to intracellularly deliver fluorescently-labeled plasmid DNA encoding GFP. However, GFP expression was only detected in 0.1% of cells (see Table 7.1 of WO/2016/161516), strongly suggesting that the internalized plasmid DNA remained trapped in endosomes without gaining access to the cytosolic/nuclear compartment. Example 7.3 of WO/2018/068135 revisited the ability of a plurality of first- and second-generation synthetic peptide shuttle agents to successful transfect cells with a GFP-encoding plasmid. The results were similar—GFP expression was detected in less than 1% of cells for all shuttle agents (see Table 7.2 of WO/2018/068135). These results suggested that synthetic peptide shuttle agents are not suitable for transducing DNA/RNA to the cytosol/nucleus of eukaryotic cells.

Several strategies were undertaken to attempt to deliver DNA/RNA cargoes to the cytosolic/nuclear compartment without success. Interestingly, in experiments attempting to co-transduce both GFP and polynucleotide cargoes simultaneously, it was observed that the presence of the polynucleotide diminished the transduction efficiency of the GFP cargoes in a concentration-dependent manner. Hypothesizing that the inhibitory effect of the polynucleotide was perhaps due to the negatively charged phosphate backbone, we attempted neutralizing the negative charges by coating the polynucleotides with small positively charged molecules prior to transduction. Small positively-charged molecules that were tried included 1,3-diaminoguanidine monohydrochloride; 3,5-diamino-1,2,4-triazole; guanidine hydrochloride; and L-arginine amide dihydrochloride at concentration ranging from 100 nM to 10 mM. However, these strategies failed to significantly improve cytosolic/nuclear delivery of DNA/RNA cargoes, with endosomal entrapment continuing to be problematic, potentially suggesting that more than mere charge neutralization was required for shuttle agent-medicated transduction.

### Example 4: Synthetic Peptide Shuttle Agents Enable Intracellular Delivery of FITC-Labeled Charge-Neutral Polynucleotide Analogs

Phosphorodiamidate morpholino oligomers (PMOs) are short single-stranded polynucleotide analogs useful as antisense oligonucleotdes for modifying gene expression via steric hindrance. Their molecular structures contain DNA/RNA nucleobases attached to a backbone of methylenemorpholine rings linked through phosphorodiamidate groups. Because of their charge-neutral structures, many intracellular delivery systems developed for DNA/RNA (e.g., cationic lipids; electrostatic coupling with cationic cell-penetrating peptides) are not suitable for intracellular delivery of PMOs. As such, modified forms of PMOs have been developed for intracellular delivery, including covalently linking the PMOs to eight guanidinium head groups (Vivo-morpholinos) or to cell-penetrating peptides (PPMOs). However, other than direct injection strategies, there have been few reports of successful cytosolic delivery of unmodified PMOs capable of modifying gene expression.

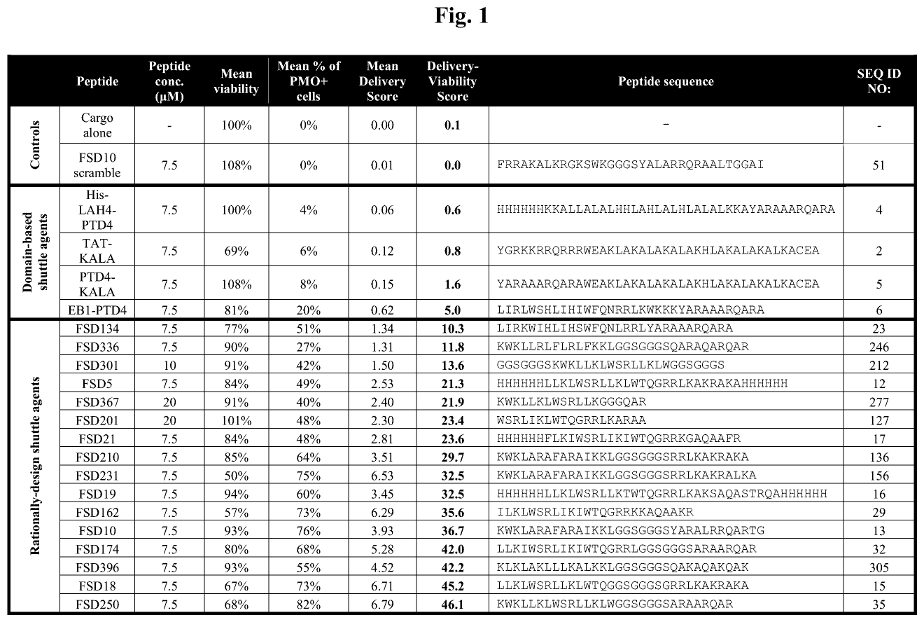

To explore whether synthetic peptide shuttle agents can deliver PMOs intracellularly, HeLa cells were exposed for 5 minutes to plain RPMI media containing 6 μM of a 25-mer PMO molecule covalently labeled with a fluorophore (“PMO-FITC”; SEQ ID NO: 348) in the presence of 7.5, 10 or 20 μM of representative members of first- and second-generation synthetic peptide shuttle agents. The results are shown in FIG. 1, in which mean cellular viability (“Mean viability”) and “Mean % of PMO+ cells” (number of viable cells positive for the PMO-FITC cargo) were evaluated by flow cytometry as described in WO/2018/068135. “Mean Delivery score” provides a further indication of the total amount of cargo (PMO-FITC) that was delivered per cell amongst all cargo-positive cells and was calculated by multiplying the mean fluorescence intensity (of at least duplicate samples) measured for the viable PMO-FITC+ cells, by the mean percentage of viable PMO-FITC+ cells, divided by 100,000. Finally, a “Delivery-Viability Score” was calculated for each peptide as the Mean viability multiplied by the Mean Delivery Score multiplied by 10, enabling a ranking of the shuttle agents in terms of both their transduction activity and toxicity. The rows in FIG. 1 are sorted from lowest to highest in terms of their Delivery-Viability Scores.

Included as negative controls were “cargo alone” (cells incubated with PMO-FITC in the absence of peptide) and “FSD10 scramble” (a control peptide having the same amino acid composition of the shuttle agent FSD10, except that the primary amino acid sequence is “shuffled” to destroy the cationic amphipathic structure common to all shuttle agents). The results in FIG. 1 show that second generation shuttle agents, which were designed for transduction of protein cargoes, generally outperformed first generation shuttle agents—both in terms of transduction efficiency (Mean % of PMO+ cells) and the average amount of cargo delivered into each cell (Delivery Score). The latter is particularly advantageous for gene expression modification purposes given that the effect of PMOs and other antisense oligonucleotide analogs depends on intracellular concentration. The first generation shuttle agents CM18-Penetratin-cys, CM18-TAT, and His-CM18-PTD4 that were included in the experiment exhibited higher toxicities (i.e., mean viabilities below 50%) at the lowest concentration tested (7.5 μM) and were thus excluded from FIG. 1. This result was unexpected since such first generation shuttle agents have been used at higher concentrations for delivering GFP-NLS in the same HeLa cells without a comparable negative impact on viability. The increased toxicity could not be attributed solely to PMO-FITC cargo toxicity since many other shuttle agents tested showed much higher Mean % of PMO+ cells and Mean PMO Delivery Scores without the same level of toxicity. These results suggested that toxicities may be due to interactions between each shuttle agent and the PMO-FITC cargo, and that shuttle agent/cargo “matching” may be considered for toxicity purposes, as well as transduction activity.

Collectively, the results in FIG. 1 show that synthetic peptide shuttle agents are able to deliver PMO-FITC cargoes intracellularly, with second generation shuttles generally outperforming first generation shuttles in terms of transduction efficiency, the amount of cargo delivered per cell, and toxicity.

### Example 5: Synthetic Peptide Shuttle Agent FSD10 Transduces Antisense PMO to the Cytosol Enabling Knock-Down of GFP Gene Expression in HeLa Cells

Endosomal entrapment was found to be problematic for shuttle agent-mediated transduction of naked DNA/RNA cargoes (Example 3). The flow cytometry results presented in Example 4 and FIG. 1 consider cells positive for the PMO-FITC cargo regardless of whether the cargo is cytosolic/nuclear or endosomally trapped. Furthermore, recent reports from other groups have shown that hydrophobic fluorophores (e.g., tetramethylrhodamine) may interact with lipid bilayers and enhance membrane destabilization (Brock et al., 2018). Thus, experiments were performed to confirm that shuttle agents can deliver unlabelled PMO cargoes to the cytosolic/nuclear compartment in a biologically active form to knock-down expression of a target gene.

A “HeLa plex TetO GFPd” cell line was created, consisting of HeLa cells stably expressing a variant of GFP (“GFPd”) engineered to have a shorter half-life of about 2 hours. HeLa plex TetO GFPd cells were exposed for 5 minutes to plain RPMI media containing different concentrations of either an antisense PMO molecule designed to knock-down expression of GFPd (“PMO-GFP”; SEQ ID NO: 345), or an off-target antisense PMO molecule targeting GLI1 expression (“PMO-Gli1”; SEQ ID NO: 346), and with or without a synthetic peptide shuttle agent. As a further control, siRNAs having the same sequence as the anti-sense (“siRNA-GFP”; SEQ ID NOs: 349) and non-specific (“siRNA-Gli1”; SEQ ID NO: 350) PMO molecules were included and delivered via a commercial cationic lipid-based delivery system (Lipofectamine™ RNAiMax). Following transduction/transfection, cells were washed, cultured in growth medium, and then analyzed by flow cytometry to evaluate the effects on GFPd expression at appropriates times to observe knock-down effects (5 hours for PMO treatments or 48 hours for siRNA treatments).

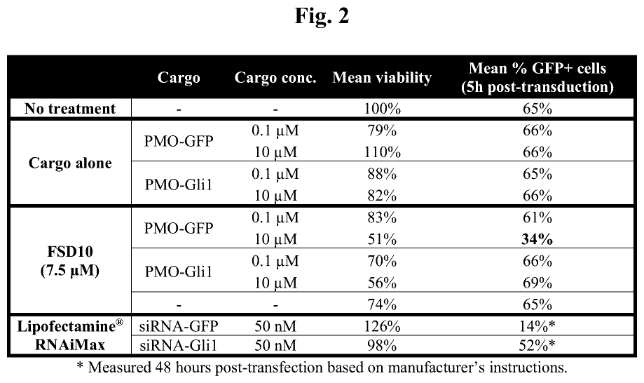

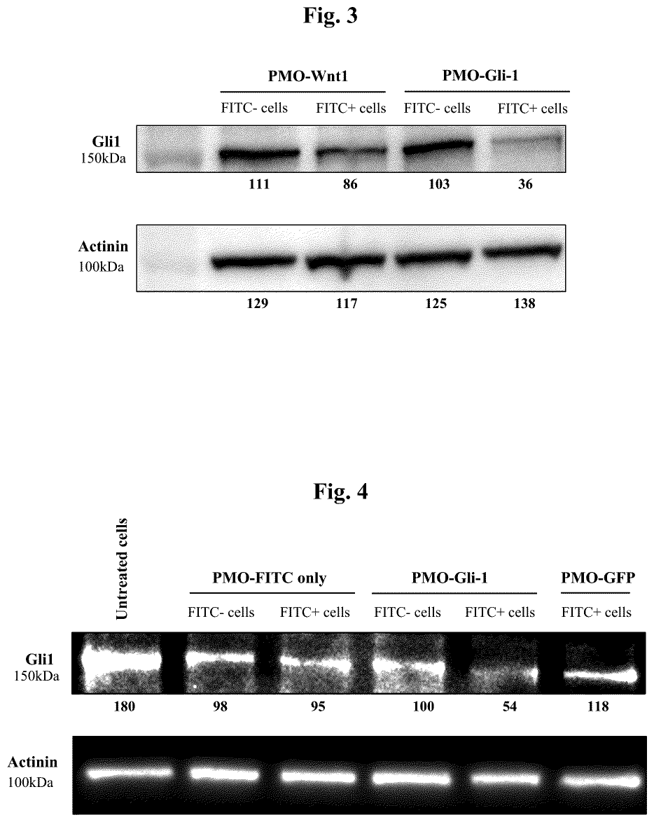

As shown in FIG. 2, untreated HeLa plex TetO GFPd cells had a baseline of 65% mean GFP+ cells and exposing the cells to the PMO cargoes alone (without shuttle agent) resulted in no change in this regard. Interestingly, exposing the cells to 10 μM of the PMO-GFP cargo in the presence of 7.5 μM of the shuttle agent FSD10 resulted in a decrease in the percentage of GFP+ cells to 34%. The effect was specific to the PMO-GFP cargo, since no effect on GFPd expression was observed with the negative control PMO-non-specific cargo. Finally, the effect was dose dependant since the knock-down in GFPd expression was lost by reducing the PMO-GFP cargo concentration to 0.1 μM.

### Example 6: Synthetic Peptide Shuttle Agent FSD250 Transduces Antisense PMO to the Cytosol Enabling Knock-Down of Gli1 Expression in DU145 Cells

Human DU145 cells were exposed for 5 minutes to plain RPMI media containing 6 μM of either an antisense PMO molecule designed to knock-down expression of the Gli1 protein (“PMO-Gli1”; SEQ ID NO: 346), or an antisense PMO molecule designed to knock-down expression of the Wnt1 protein (“PMO-Wnt1”; SEQ ID NO: 347), in the presence of 5 μM of the synthetic peptide shuttle agent FSD250. A tracer PMO-FITC molecule (6 μM) was also included in both conditions to enable the fluorescence-activated cell sorting (FACS) of transduced cell from non-transduced cells within the same cell population. At 48 hours post-transduction, the cells were analyzed by flow cytometry and then separated by FACS into a FITC-positive population and a FITC-negative population.

For transduction of PMO-Gli/PMO-FITC, mean % PMO-FITC+ cells was 37% and viability was For transduction of PMO-Wnt1/PMO-FITC, mean % PMO-FITC+ cells was 36.8% and viability was 75.7%. For untreated cells, mean % PMO-FITC+ cells was 0.6% and viability was 91.3%.

FITC+ and FITC− cell populations were then lysed, resolved by SDS-PAGE, and subjected to Western blot analysis using an anti-Gli1 polyclonal antibody, an anti-Actinin polyclonal antibody as a loading control, as well as appropriate enzyme-conjugated secondary antibodies. The results are shown in FIG. 3, including densitometry scanning values for each band. FIG. 3 clearly shows that Gli1 protein expression was knocked-down by shuttle agent-mediated intracellular delivery of antisense PMO-Wnt1 and antisense PMO-Gli1 in transduced cells (“FITC+ cells”) as compared to non-transduced cells (“FITC− cells”). As expected, the Gli1 knock-down effect was stronger with the direct knock-down of Gli1 mRNA with PMO-Gli1 than with the indirect knock-down of Gli1 via knock-down of Wnt1 mRNA, which is part of the same signalling pathway.

### Example 7: Synthetic Peptide Shuttle Agent FSD250 Transduces Antisense PMO to the Cytosol Enabling Knock-Down of Gli1 Rxpression in DU145 Cells

Human DU145 cells were exposed for 5 minutes to plain RPMI media containing 6 μM of either PMO-Gli1 (SEQ ID NO: 346) or PMO-GFP (SEQ ID NO: 345) in the presence of 3.75 μM of the synthetic peptide shuttle agent FSD250. A tracer PMO-FITC molecule was also included in both conditions (as well as alone as an additional negative control) to enable separation by FACS of transduced cells from non-transduced cells. At 48 hours post-transduction, the cells were separated by FACS into a FITC-positive population and a FITC-negative population.

For transduction of PMO-FITC only, mean % PMO-FITC+ cells was 44.1% and viability was 77.7%. For transduction of PMO-Gli1/PMO-FITC, mean % PMO-FITC+ cells was 36.0% and viability was 61.1%. For transduction of PMO-GFP/PMO-FITC, mean % PMO-FITC+ cells was 48.9% and viability was 77.0%. For untreated cells, mean % PMO-FITC+ cells was 0.1% and viability was 84.5%.

FITC+ and FITC− cell populations were then lysed, resolved by SDS-PAGE, and subjected to Western blot analysis using an anti-Gli1 polyclonal antibody, an anti-Actinin polyclonal antibody as a loading control, as well as appropriate enzyme-conjugated secondary antibodies. The results are shown in FIG. 4, including relative densitometry scanning values for each band each normalized to their respective actinin loading control band. FIG. 4 clearly shows that Gli1 protein expression was knocked-down by shuttle agent-mediated intracellular delivery of antisense PMO-Gli1, but not by cells transduced with tracer PMO-FITC alone or with PMO-GFP.

### Example 8: Large-Scale Screening of Candidate Peptide Shuttle Agents for Propidium Iodide (PI) and GFP-NLS Transduction Activity

A proprietary library of over 300 candidate peptide shuttle agents was screened in parallel for both propidium iodide (PI; a small molecule cargo) and GFP-NLS transduction activity in HeLa cells using flow cytometry as generally described in Example 1. PI was used a cargo because it exhibits 20- to 30-fold enhanced fluorescence and a detectable shift in maximum excitation/emission spectra only after being bound to genomic DNA—a property that makes it particularly suitable to distinguish endosomally-trapped cargo from endosomally-escaped cargo having access to the cytosolic/nuclear compartment. Thus, intracellular delivery and endosomal escape could both be measurable by flow cytometry since any PI that remained trapped in endosomes would not reach the nucleus and would exhibit neither the enhanced fluorescence nor the spectra shift.

Due to the large number of peptides screened, negative controls were performed in parallel for each experimental batch and included a “no treatment” (NT) control in which the cells were not exposed to shuttle peptide or cargo, as well as a “cargo alone” control in which cells were exposed to the cargo in the absence of shuttle agent. Results are shown in FIG. 5, in which “transduction efficiency” refers to the percentage of all viable cells that are positive for the cargo (PI or GFP-NLS). “Mean Delivery score” provides a further indication of the total amount of cargo that was delivered per cell, amongst all cargo-positive cells. Mean PI or GFP-NLS delivery score was calculated by multiplying the mean fluorescence intensity (of at least duplicate samples) measured for the viable PI+ or GFP+ cells by the mean percentage of viable PI+ or GFP+ cells, divided by 100,000 for GFP delivery or by 10,000 for PI delivery. The Mean Delivery Scores for PI and GFP-NLS for each candidate shuttle agent was then normalized by dividing by the Mean Delivery Score for the “cargo alone” negative control performed in parallel for each experimental batch. Thus, the “Norm. Mean Delivery Score” in FIG. 5 represents the fold-increase in Mean Delivery Score over the “cargo alone” negative control.

The batch-to-batch variation observed for the negative controls was relatively small for GFP-NLS but was appreciably higher with PI as cargo. For example, the variation in transduction efficiency for the “cargo alone” negative control ranged from 0.4% to 1.3% for GFP-NLS and from 0.9% to 6.3% for PI. Furthermore, transduction efficiencies for several negative control peptides (i.e., peptides known to have low or no GFP transduction activity) tested in parallel (e.g., FSD174 Scramble; data not shown) sometimes gave lower transduction efficiencies for PI (but not for GFP-NLS) than the “cargo alone” negative control, in some cases by as much as 5%, perhaps due to non-specific interactions between PI and the peptides. This phenomenon was not observed for GFP-NLS transduction experiments. The foregoing suggested that the shuttle agent transduction efficiencies at least for PI may be more appropriately compared to that of a negative control peptide rather than to the “cargo alone” condition.

Included amongst the candidate peptide shuttle agents in FIG. 5 having a mean PI transduction efficiency of at least 20% were peptides having lengths of less than 20 residues: FSD390 (17 aa), FSD367 (19 aa), and FSD366 (18 aa). Also included amongst the candidate peptide shuttle agents in having a mean PI transduction efficiency of at least 20% were peptides comprising either non-physiological amino acid analogs (e.g., FSD435, which corresponds to FSD395 except for lysine residues (K) being replaced with L-2,4-diaminobutyric acid residues) or chemical modifications (e.g., FSD438, which corresponds to FSD10 except for an N-terminal octanoic acid modification; FSD436, which corresponds to FSD222 except for phenylalanine residues (F) being replaced with (2-naphthyl)-L-alanine residues; FSD171, which corresponds to FSD168 except having an N-terminal acetyl group and a C-terminal cysteamide group. These results confirm the robustness of the peptide shuttle agent platform technology to tolerate the use of non-physiological amino acids or analogs thereof in place of physiological amino acids and/or chemical modifications.

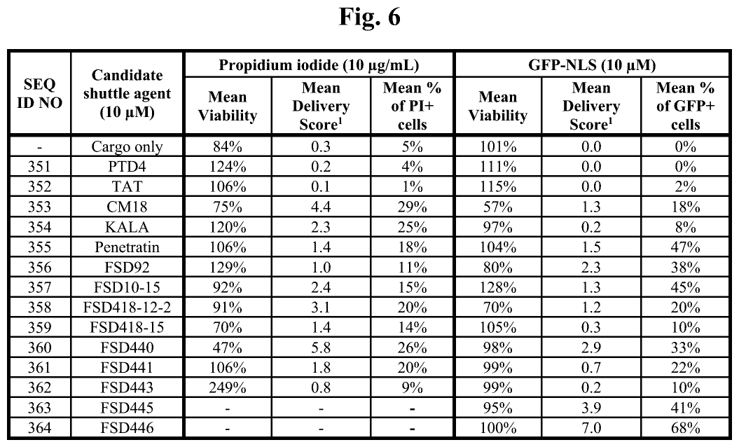

Additional screening assays were performed with further shuttle agents, as shown in FIG. 6. Amongst the results were several active shuttle agents that are truncations of longer shuttle agents previously shown to have transduction activity: FSD10-15 (15aa) and FSD418-12-2 (12 aa), FSD418 (15aa), CM18 (18 aa), and Penetratin (16 aa).

### Example 9: Synthetic Peptide Shuttle Agents Efficiently Transduce PMO and PNAs in Hela Cells

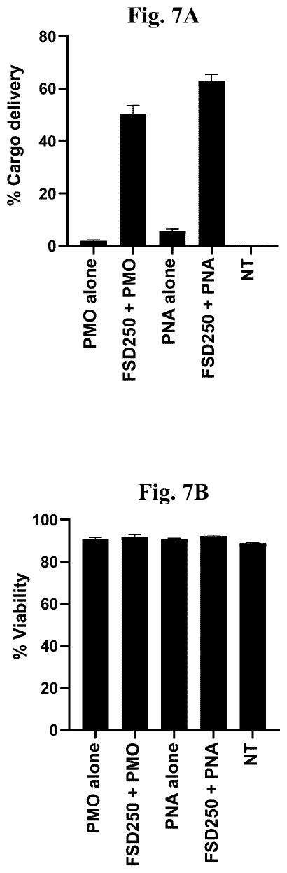

HeLa cells were transduced with 10 μM of PMO-FITC or fluorescently-labeled Peptide Nucleic Acid (PNA) (PNA TelC-Alexa 488; cat. no. F1004; PNA BIO Inc.) as generally described in the transduction protocol described in Example 1 with a few modifications. The shuttle peptide used was FSD250 (5 μM) and cells were contacted with the cargo and shuttle agent for two minutes, and cells were analyzed after a 1-hour incubation by flow cytometry. Furthermore, PNA was resolubilized in water instead of the manufacturer-recommended dimethylformamide (DMF) since the inclusion of DMF in culture media resulted in cell viabilities of below 50%. The cargo transduction results in FIG. 7A demonstrate that FSD250 greatly increased intracellular delivery of both PMO (“FSD250+PMO” delivery) and PNA (“FSD250+PNA”) cargoes, as compared to in the absence of the shuttle agent (“PMO alone” and “PNA alone”; “NT”=untreated cells). Cell viability remained high in all conditions tested, as shown in FIG. 7B. A parallel experiment using FSD250 to attempt to transduce fluorescently-labeled siRNA as a cargo resulted in only 5% intracellular delivery (data not shown). These results suggest that, in addition to PMOs, synthetic peptide shuttle agents can also rapidly and efficiently transduce other non-anionic polynucleotide analogs such as PNAs.

### Example 10: Effect of Naked DNA/RNA on Shuttle Agent-Mediated Transduction of PMO Cargoes

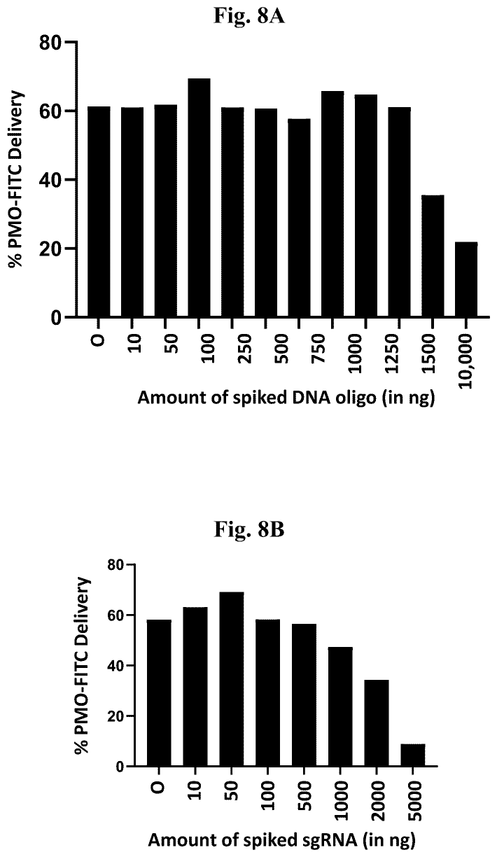

Although naked DNA/RNA cargoes are shown to themselves be poor cargoes of synthetic peptide shuttle agents (Examples 3 and 9), the present Example evaluates their potential dominant negative effect in trans on shuttle agent-mediated transduction of PMO cargoes. Briefly, RH-30 cells (150,000 cells/well in 24-well dish) were contacted with a delivery mix of 6 μM of a PMO-FITC and of 5 μM of the synthetic peptide shuttle agent FSD250 for 2 minutes in RPMI, in the presence of increasing amounts of a DNA oligonucleotide or an sgRNA spiked in medium. Cells were then washed, incubated in growth medium and then collected for analysis by flow cytometry after 1 h. The results in FIG. 8 show that reduced PMO-FITC transduction efficiency was observed in the presence of 1.5 lag of DNA oligo (3 μg/mL) (FIG. 8A) and 2 μg of sgRNA (4 μg/mL) (FIG. 8B). In all conditions tested, cell viability remained above 75%.

### Example 11: Comparison of PMO Cargo Transduction by First and Second Generation Synthetic Peptide Shuttle Agents

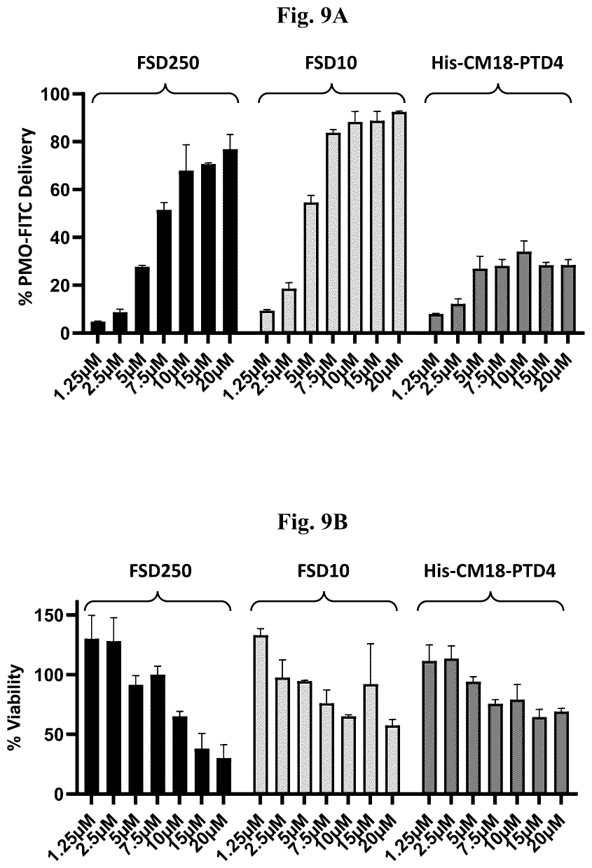

In general, second-generation synthetic peptide shuttle agents exhibit higher cargo transduction efficiencies than first generation shuttle agents. The present Example compares the PMO transduction activity of a prototypical CPD-comprising first generation shuttle agent with that of two rationally-designed second generation synthetic peptide shuttle agents. Briefly, RH-30 cells (20,000 cells/well in 96-well dish) were contacted with a delivery mix of 6 μM of a PMO-FITC and increasing concentrations of the first-generation shuttle agent His-CM18-PTD4 or two CPD-lacking second generation synthetic peptide shuttle agents (FSD250 and FSD10) for 2 minutes in RPMI. Cells were washed, incubated in complete medium and then collected for analysis by flow cytometry after 1 h. PMO-FITC transduction efficiency is shown in FIG. 9A and cell viability is shown in FIG. 9B. The results in FIG. 9A show that FSD250 and FSD10 yielded higher transduction efficiencies for the PMO-FITC cargo than His-CM18-PTD4.

### Example 12: Comparison of Synthetic Peptide Shuttle Agent-Mediated PMO Transduction with Self-Internalizin2 VivoPMOs

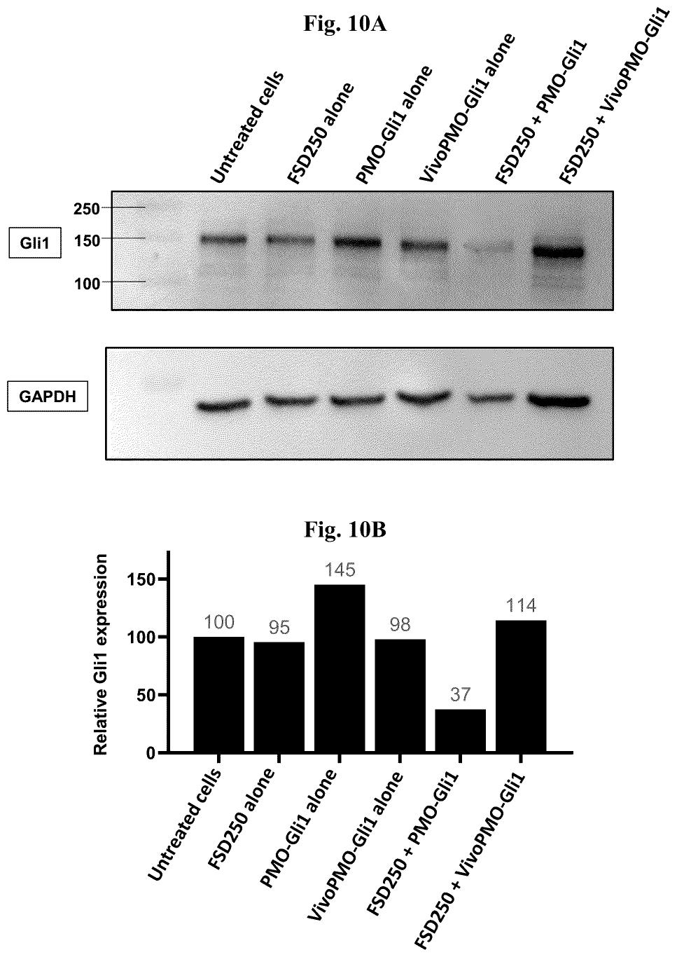

A Gli1 knock-down experiment was performed generally as described in Example 6 to compare shuttle agent-mediated transduction of an unmodified PMO versus a commercially available self-internalizing Vivo-Morpholino (VivoPMO), which is a PMO chemically modified with a terminal octa-guanidinium dendrimer to facilitate entry into cells. Briefly, RH-30 cells were contacted with a delivery mix of 6 μM of cargo (either PMO-Gli1 or VivoPMO-Gli1) in the presence or absence of 5 μM of the synthetic peptide shuttle agent FSD250 for 2 minutes in RPMI. Cells were then washed, incubated in complete medium and then collected for Gli1 protein expression analysis by Western blot after 24 h using an anti-Gli1 polyclonal Ab (Abcam ab273018, 150 kDa), an anti-GAPDH Ab (Abcam ab181602, 37 kDa), and anti-Rabbit HRP secondary Ab. The Western blot results are shown in FIG. 10A and the corresponding densitometry scanning analysis is shown in FIG. 10B. Knockdown of Gli1 protein was only observed in cells treated with both FSD250 and the PMO-Gli1 cargo. The lack of Gli1 knockdown observed in cells exposed to VivoPMO-Gli 1 alone suggests that the 2-minute incubation time in FIG. 10 was insufficient for the VivoPMO to self-internalize. Indeed, this is consistent with the manufacturer's recommended incubation time of 2-4 hours for VivoPMOs to achieve sufficient self-internalization via endocytosis. Regardless, the results in FIG. 10 highlight the vast difference in internalization kinetics between the slow endocytosis-dependent intracellular delivery of VivoPMOs versus the rapid and efficient PMO transduction observed using synthetic peptide shuttle agents.

### Example 13: Comparison of Synthetic Peptide Shuttle Agent-Mediated PMO Transduction with Endoporter-Mediated Intracellular PMO Delivery

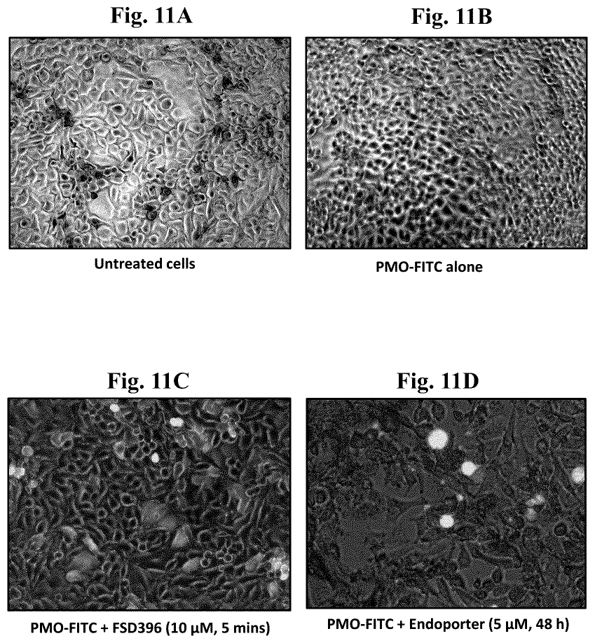

A PMO cargo delivery experiment in HeLa cells was performed to directly compare synthetic peptide shuttle agent-mediated PMO transduction with Endoporter-mediated intracellular PMO delivery. For shuttle agent-mediated transduction, HeLa cells were exposed to 10 μM of PMO-FITC in the presence of 2.5, 5, 7.5, or 10 μM of the second-generation shuttle agent FSD396 for 5 minutes in RPMI. For Endoporter-mediated delivery, HeLa cells were exposed to 10 μM of PMO-FITC in the presence of 2.5, 5, 7.5, or 10 μM of the commercially-available Endoporter' peptide (GeneTools, LLC) in growth medium for the manufacturer's recommended minimum incubation time of at least 24 hours. After a washing step, delivery results were compared by observing intracellular PMO-FITC fluorescence via immunofluorescence microscopy and results are shown in FIG. 11. HeLa cells exposed to the PMO-FITC cargo alone (FIG. 11B) exhibited only slight intracellular delivery after 48 h as compared to untreated cells (FIG. 11A). While FSD396 induced robust intracellular delivery of PMO-FITC after a 5-minute incubation (FIG. 11C), a comparable level of intracellular PMO-FITC was only achievable with the Endoporter peptide after a 48-hour incubation (FIG. 11D). Furthermore, Endoporter-treated cells exhibited undesirable morphological changes that were not observed in untreated cells (FIG. 11A), in cells treated with cargo alone (FIG. 11B), or in shuttle agent-treated cells (FIG. 11C). Large PMO-FITC aggregates were also observable in Endoporter-treated cells that could not be removed from the cells via a washing step.

### Example 14: Gli1 Knockdown Triggers Increased Apoptosis in a BCC Cell Line but not in Normal Human Skin Cell Line

Gorlin syndrome, also known as Nevoid Basal Cell Carcinoma or Basal Cell Carcinoma Nevus Syndrome (BCCNS), is a genetic disease associated with aberrant Hedgehog (Hh) pathway signalling leading to the frequent growth of basal cell carcinomas (BCCs) on face, hands, back and neck. Patients suffering from Gorlin syndrome may develop up to 30 lesions per year originating from the basal cell layer of the skin situated between the epidermis and the dermis. Gorlin patients have genetic mutations which lead to constitutive activation of the Hh pathway. Gli1 is the transcription factor responsible for the expression of determinants of the Hh pathway and may thus be considered as a master regulator of Hh signalling.

The effect of Gli1 knockdown on two human skin cell lines was evaluated: a skin epithelial-like cell line originating from normal human skin (NCTC-2544) and a human basal cell carcinoma cell line (UW-BCC1). The normal-derived NCTC-2544 cells and the BCC-derived UW-BCC1 cells were exposed to self-internalizing VivoPMO-Gli1 (15 μM) in complete cell culture medium for 24 or 48 h. Approximately a 60% knockdown of Gli1 protein expression was observed by Western blot after 48 h. In parallel, the percentage of cellular apoptosis was measured by flow cytometry with fluorescently-labeled Annexin-V. Interestingly, treatment with VivoPMO-Gli1 resulted in 68-72% apoptotic UW-BCC1 cells after 48 h, as compared to only 11% apoptotic cells treated with a negative control VivoPMO. In contrast, treatment with VivoPMO-Gli1 resulted in only 3-6% apoptotic NCTC-2544 cells after 48 h and only 2% apoptosis in cells treated with the negative control VivoPMO. These results support Gli1 knockdown via intracellular delivery of Gli1-specific PMOs for the treatment for basal cell carcinoma.

### Example 15: Design, Synthesis, and Shuttle Mediated-Transduction of PMOs for Gli1 Knockdown

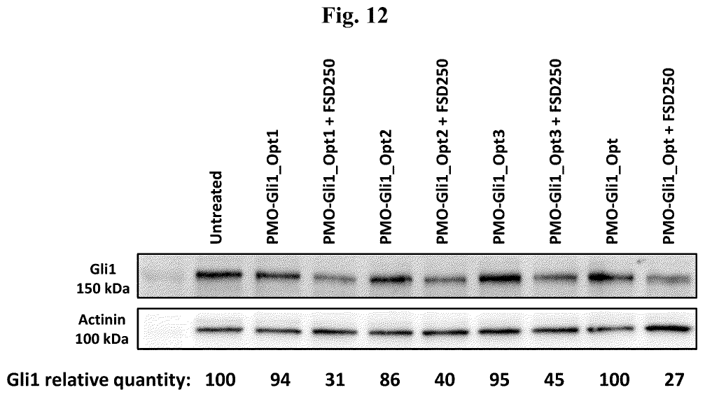

Four different PMOs were designed and synthesized targeting different regions proximal to the region or start codon of the human Gli1 gene. In ascending order of their distance from the Gli1 start codon, the four PMOs synthesized were: PMO-Gli1_Opt (binding to SEQ ID NO: 365 and straddling the Gli1 start codon); PMO-Gli1_Opt1 (binding to SEQ ID NO: 366); PMO-Gli1_Opt2 (binding to SEQ ID NO: 367); and PMO-Gli1_Opt3 (binding to SEQ ID NO: 368). RH-30 cells were transduced with each of the four PMO cargoes (6 μM) or with a negative control PMO-FITC (6 μM) with the shuttle agent FSD250, as described in Example 12. Overall transduction efficiency in the transduction experiment was approximately 80%, as estimated by flow cytometry of cells transduced with the PMO-FITC control cargo. Cells were harvested 24 hours post-delivery and knockdown of Gli1 protein expression was evaluated by Western blotting (FIG. 12). All four of the PMOs knocked down Gli1 protein expression when transduced with the shuttle agent, but minimal knockdown was observed in the absence of the shuttle agent. Densitometry analysis of the Gli1 and control Actinin bands revealed that the four PMOs knocked down Gli1 expression to 27-45% of that of untreated cells (FIG. 12). The transduction experiment was repeated with increasing concentrations of PMO-Gli1_Opt and Gli1 knockdown was found to be dose-dependent: 0.325, 0.75, 1.5, 3, and 6 μM of PMO-Gli1_Opt knocked down Gli1 levels to 48%, 43%, 34%, 33%, and 22% of that of untreated cells, respectively.

### Example 16: Shuttle Mediated-Transduction of PMOs for Gli1 Knockdown in Basal Cells of Patient-Derived Tumor Explants

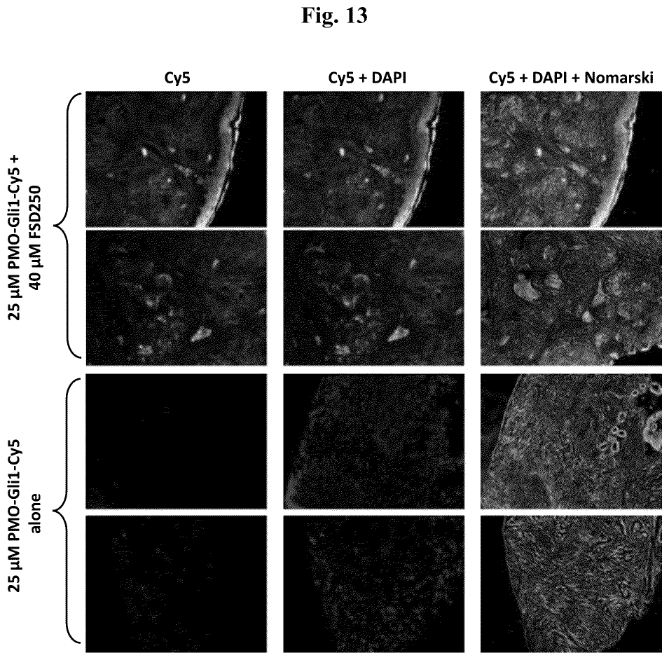

Freshly obtained basal cell carcinoma-type tumors following Mohs-type surgery were incubated in complete DMEM culture medium on a wire mesh with the surface exposed to air. In order to allow the cargo to be delivered to the epidermis and dermis, the tumors were washed with PBS 1× and the stratum corneum of the explants is permeabilized with a Pantec PLEASE™ laser according to the following parameters: pore density 2.5%, 1 pulse/pore, Array Size 14 mm, depth pores 20 μm (4.9 J/cm², 0.8 W). The explants were then divided into two halves, one half was treated with a solution of PBS 1×-2% hydroxyethyl cellulose containing 25 μM of PMO-Gli1-Cy5 and 40 μM of FSD250, while the other half was treated with the same solution containing PMO-Gli1-cy5 only (without shuttle agent; control). Following the treatment, the tumors were incubated for 4 hours at 37° C. and fixed with 4% paraformaldehyde (PFA), then treated with 30% sucrose and frozen in OCT (optimal cutting temperature). 10 μm sections were transferred to coverslips and treated with ProLong™ Diamond for fluorescence microscopy analysis. As shown in FIG. 13, increased fluorescence levels in the tumor half treated with the PMO-Gli1-cy5 and shuttle agent combination, especially at the level of the epidermis, was observed in comparison to the other tumor half treated with PMO-Gli1-Cy5 alone. Furthermore, following incubation of the tumors, the cells of the dermis and the epidermis were isolated by enzymatic treatments with thermolysin and trypsin and further cultured at 37° C. in 5% CO₂. The levels of Gli1 protein were measured by cytofluorometry at different times of culture following the fixation and labeling of the isolated dermal and epidermal cells with an anti Gli1-Alexa 647 antibody. Between 24 and 48 hours of incubation following the treatment of the tumors, the level of the Gli1 protein decreased considerably (50%) in cultured cells which were treated with the PMO-Gli1 and shuttle agent combination, measured by flow cytometry (data not shown).

### REFERENCES

Andreu et al., (1992) “Shortened cecropin A-melittin hybrids. Significant size reduction retains potent antibiotic activity”. FEBS letters 296, 190-194
Amand et al., (2012). “Functionalization with C-terminal cysteine enhances transfection efficiency of cell-penetrating peptides through dimer formation.” Biochem Biophys Res Commun 418(3): 469-474.
Boman et al., (1989) Antibacterial and antimalarial properties of peptides that are cecropin-melittin hybrids. FEBS letters 259, 103-106.
Brock et al., (2018) “Efficient cell delivery mediated by lipid-specific endosomal escape of supercharged branched peptides”. Traffic 19(6):421-435. doi: 10.1111/tra.12566.
Del'Guidice et al., (2018) “Membrane permeabilizing amphiphilic peptide delivers recombinant transcription factor and CRISPR-Cas9/Cpf1 ribonucleoproteins in hard-to-modify cells”. PLoS ONE 13(4): e0195558.
Drin et al., (2003). “Studies on the internalization mechanism of cationic cell-penetrating peptides.” J Biol Chem 278(33): 31192-31201.
Eisenberg et al., (1982). “The helical hydrophobic moment: a measure of the amphiphilicity of a helix”. Nature 299, 371— 374.
El-Andaloussi et al., (2007). “A novel cell-penetrating peptide, M918, for efficient delivery of proteins and peptide nucleic acids.” Mol Ther 15(10): 1820-1826.
El-Sayed et al., (2009). “Delivery of macromolecules using arginine-rich cell-penetrating peptides: ways to overcome endosomal entrapment.” AAPS J 11(1): 13-22.
Elmquist et al., (2001). “VE-cadherin-derived cell-penetrating peptide, pVEC, with carrier functions.” Exp Cell Res 269(2): 237-244.
Erazo-Oliveras et al., (2014) “Protein delivery into live cells by incubation with an endosomolytic agent.” Nat Methods. (8):861-7.
Fawell et al., (1994). “Tat-mediated delivery of heterologous proteins into cells.” Proc Natl Acad Sci USA 91(2): 664-668.
Fominaya et al., (1998). “A chimeric fusion protein containing transforming growth factor-alpha mediates gene transfer via binding to the EGF receptor.” Gene Ther 5(4): 521-530.
Fominaya, J. and W. Wels (1996). “Target cell-specific DNA transfer mediated by a chimeric multidomain protein. Novel non-viral gene delivery system.” J Biol Chem 271(18): 10560-10568.
Glover et al., (2009). “Multifunctional protein nanocarriers for targeted nuclear gene delivery in nondividing cells.” FASEB J 23(9): 2996-3006.
Gottschalk et al., (1996). “A novel DNA-peptide complex for efficient gene transfer and expression in mammalian cells.” Gene Ther 3(5): 448-457.
Green, M. and P. M. Loewenstein (1988). “Autonomous functional domains of chemically synthesized human immunodeficiency virus tat trans-activator protein.” Cell 55(6): 1179-1188.
Hallbrink et al., (2001). “Cargo delivery kinetics of cell-penetrating peptides.” Biochim Biophys Acta 1515(2): 101-109.
Herce, H. D. and A. E. Garcia (2007). “Molecular dynamics simulations suggest a mechanism for translocation of the HW-1 TAT peptide across lipid membranes.” Proc Natl Acad Sci USA 104(52): 20805-20810.
Ho et al., (2001). “Synthetic protein transduction domains: enhanced transduction potential in vivo.” Cancer Research 61: 474-477.
Ilfeld and Yaksh (2009). “The End of Postoperative Pain—A Fast-Approaching Possibility? And, if So, Will We Be Ready?” Regional Anesthesia and Pain Medicine 34(2): 85-87.
Kakudo et al., (2004). “Transferrin-modified liposomes equipped with a pH-sensitive fusogenic peptide: an artificial viral-like delivery system.” Biochemistry 43(19): 5618-5628.
Kichler et al., (2006). “Cationic amphipathic histidine-rich peptides for gene delivery.” Biochim Biophys Acta 1758(3): 301-307.
Kichler et al., (2003). “Histidine-rich amphipathic peptide antibiotics promote efficient delivery of DNA into mammalian cells”. Proc Natl Acad Sci USA. 2003 Feb. 18; 100(4): 1564-1568.
Krishnamurthy et al., (2019). “Engineered amphiphilic peptides enable delivery of proteins and CRISPR-associated nucleases to airway epithelia”. Nature Communications. 10(1): 4906. doi: 10.1038/s41467-019-12922-y.
Kwon, et al., (2010). “A Truncated HGP Peptide Sequence That Retains Endosomolytic Activity and Improves Gene Delivery Efficiencies”. Mol. Pharmaceutics, 7:1260-65.
Lamiable et al., (2016). “PEP-FOLD3: faster de novo structure prediction for linear peptides in solution and in complex” Nucleic Acids Res. 44(W1):W449-54.
Li et al., (2004). “GALA: a designed synthetic pH-responsive amphipathic peptide with applications in drug and gene delivery.” Adv Drug Deliv Rev 56(7): 967-985.
London, E. (1992). “Diphtheria toxin: membrane interaction and membrane translocation.” Biochim Biophys Acta 1113(1): 25-51.
Lorieau et al., (2010). “The complete influenza hemagglutinin fusion domain adopts a tight helical hairpin arrangement at the lipid:water interface.” Proc Natl Acad Sci USA 107(25): 11341-11346.
Luan et al., (2015). “Peptide amphiphiles with multifunctional fragments promoting cellular uptake and endosomal escape as efficient gene vectors.” J. Mater. Chem. B, 3: 1068-1078.
Mahlum et al., (2007). “Engineering a noncarrier to a highly efficient carrier peptide for noncovalently delivering biologically active proteins into human cells.” Anal Biochem 365(2): 215-221.
Midoux et al., (1998). “Membrane permeabilization and efficient gene transfer by a peptide containing several histidines.” Bioconjug Chem 9(2): 260-267.
Montrose et al., (2013). “Xentry, a new class of cell-penetrating peptide uniquely equipped for delivery of drugs.” Sci Rep 3: 1661.
Morris, M. C., L. Chaloin, M. Choob, J. Archdeacon, F. Heitz and G. Divita (2004). “Combination of a new generation of PNAs with a peptide-based carrier enables efficient targeting of cell cycle progression.” Gene Ther 11(9): 757-764.
Morris et al., (2001). “A peptide carrier for the delivery of biologically active proteins into mammalian cells.” Nat Biotechnol 19(12): 1173-1176.
Mól et al., (2018) “NetWheels: A web application to create high quality peptide helical wheel and net projections.” bioRxiv 416347; doi: https://doi.org/10.1101/416347 (preprint)
O'Keefe, D. O. (1992). “Characterization of a full-length, active-site mutant of diphtheria toxin.” Arch Biochem Biophys 296(2): 678-684.
Parente et al., (1990). “Mechanism of leakage of phospholipid vesicle contents induced by the peptide GALA.” Biochemistry 29(37): 8720-8728.
Perez et al., (1992). “Antennapedia homeobox as a signal for the cellular internalization and nuclear addressing of a small exogenous peptide.” J Cell Sci 102 (Pt 4): 717-722.
Salomone et al., (2012). “A novel chimeric cell-penetrating peptide with membrane-disruptive properties for efficient endosomal escape.” J Control Release 163(3): 293-303.
Schuster et al., “Multicomponent DNA carrier with a vesicular stomatitis virus G-peptide greatly enhances liver-targeted gene expression in mice.” Bioconjug Chem 10(6): 1075-1083.
Shaw et al., (2008). “Comparison of protein transduction domains in mediating cell delivery of a secreted CRE protein.” Biochemistry 47(4): 1157-1166.
Shen et al., (2014) “Improved PEP-FOLD approach for peptide and miniprotein structure prediction”. J. Chem. Theor. Comput. 10:4745-4758.
Tan et al., (2012). “Truncated peptides from melittin and its analog with high lytic activity at endosomal pH enhance branched polyethylenimine-mediated gene transfection.” J Gene Med 14(4): 241-250.
Thériault et al., “Differential modulation of Nav1.7 and Nav1.8 channels by antidepressant drugs.” European Journal of Pharmacology (2015) 764: 395-403.
Thévenet et al., “PEP-FOLD: an updated de novo structure prediction server for both linear and disulfide bonded cyclic peptides.” Nucleic Acids Res. 2012. 40, W288-293.
Uherek et al., (1998). “A modular DNA carrier protein based on the structure of diphtheria toxin mediates target cell-specific gene delivery.” J Biol Chem 273(15): 8835-8841.
Varkouhi et al., “Endosomal escape pathways for delivery of biologicals.” J Control Release 151(3): 220-228.
Veach et al., (2004). “Receptor/transporter-independent targeting of functional peptides across the plasma membrane.” J Biol Chem 279(12): 11425-11431.
Vives et al., (1997). “A truncated HW-1 Tat protein basic domain rapidly translocates through the plasma membrane and accumulates in the cell nucleus.” J Biol Chem 272(25): 16010-16017.<
Wyman et al., (1997). “Design, synthesis, and characterization of a cationic peptide that binds to nucleic acids and permeabilizes bilayers.” Biochemistry 36(10): 3008-3017.
Zhou et al., (2009). “Generation of induced pluripotent stem cells using recombinant proteins.” Cell Stem Cell 4(5): 381-384.

1. A composition comprising a non-anionic polynucleotide analog cargo for intracellular delivery and a synthetic peptide shuttle agent that is independent from, or is not covalently linked to, said non-anionic polynucleotide analog cargo, the synthetic peptide shuttle agent being a peptide comprising an amphipathic alpha-helical motif having both a positively-charged hydrophilic outer face and a hydrophobic outer face, wherein synthetic peptide shuttle agent increases cytosolic/nuclear delivery of said non-anionic polynucleotide analog cargo in eukaryotic cells as compared to in the absence of the synthetic peptide shuttle agent.

2. The composition of claim 1, wherein the non-anionic polynucleotide analog cargo is a charge-neutral or cationic antisense synthetic oligonucleotide (ASO).

3. The composition of claim 1 or 2, wherein the non-anionic polynucleotide analog cargo is:
(a) a charge-neutral polynucleotide analog cargo having a phosphorodiamidate backbone, an amide (e.g., peptide) backbone, a methylphosphonate backbone, a neutral phosphotriester backbone, a sulfone backbone, or a triazole backbone; or
(b) a cationic polynucleotide analog cargo having an aminoalkylated phosphoramidate backbone, a guanidinium backbone, an S-methylthiourea backbone, or a nucleosyl amino acid (NAA) backbone.

4. The composition of any one of claims 1 to 3, wherein the non-anionic polynucleotide analog cargo is a phosphorodiamidate morpholino oligomer (PMO), a peptide nucleic acid (PNA), a methylphosphonate oligomer, or a short interfering ribonucleic neutral oligonucleotide (siRNN).

5. The composition of any one of claims 1 to 4, wherein the non-anionic polynucleotide analog cargo is a 5- to 50-mer, a 5-mer to 75-mer, or a 5-mer to 100-mer.

6. The composition of any one of claims 1 to 5, wherein the non-anionic polynucleotide analog cargo is not covalently linked to a cell-penetrating peptide, octa-guanidine dendrimer, or other intracellular delivery moiety.

7. The composition of any one of claims 1 to 6, wherein the shuttle agent is:
(1) a peptide at least 12, 13, 14, 15, 16, 17, 18, 19, or 20 amino acids in length comprising
(2) an amphipathic alpha-helical motif having
(3) a positively-charged hydrophilic outer face, and a hydrophobic outer face, wherein at least five of the following parameters (4) to (15) are respected:
(4) the hydrophobic outer face comprises a highly hydrophobic core consisting of spatially adjacent L, I, F, V, W, and/or M amino acids representing 12 to 50% of the amino acids of the peptide, based on an open cylindrical representation of the alpha-helix having 3.6 residues per turn;
(5) the peptide has a hydrophobic moment (μ) of 3.5 to 11;
(6) the peptide has a predicted net charge of at least +3 or +4 at physiological pH;
(7) the peptide has an isoelectric point (pI) of 8 to 13;
(8) the peptide is composed of 35% to 65% of any combination of the amino acids: A, C, G, I, L, M, F, P, W, Y, and V;
(9) the peptide is composed of 0% to 30% of any combination of the amino acids: N, Q, S, and T;
(10) the peptide is composed of 35% to 85% of any combination of the amino acids: A, L, K, or R;
(11) the peptide is composed of 15% to 45% of any combination of the amino acids: A and L, provided there being at least 5% of L in the peptide;
(12) the peptide is composed of 20% to 45% of any combination of the amino acids: K and R;
(13) the peptide is composed of 0% to 10% of any combination of the amino acids: D and E;
(14) the difference between the percentage of A and L residues in the peptide (% A+L), and the percentage of K and R residues in the peptide (K+R), is less than or equal to 10%; and
(15) the peptide is composed of 10% to 45% of any combination of the amino acids: Q, Y, W, P, I, S, G, V, F, E, D, C, M, N, T and H.

8. The composition of claim 7, wherein:
(a) the shuttle agent respects at least six, at least seven, at least eight, at least nine, at least ten, at least eleven, or respects all of parameters (4) to (15);
(b) the shuttle agent is a peptide having a minimum length of 12, 13, 14, 15, 16, 17, 18, 19, 20, 21, 22, 23, 24, 25, 26, 27, 28, 29, or 30 amino acids, and a maximum length of 40, 41, 42, 43, 44, 45, 46, 47, 48, 49, 50, 51, 52, 53, 54, 55, 60, 65, 70, 80, 90, 100, 110, 120, 130, 140, or 150 amino acids;
(c) said amphipathic alpha-helical motif has a hydrophobic moment (μ) between a lower limit of

3. 5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, 10.4, 10.5, 10.6, 10.7, 10.8, 10.9, or 11.0;
(d) said amphipathic alpha-helical motif comprises a positively-charged hydrophilic outer face comprising: (i) at least two, three, or four adjacent positively-charged K and/or R residues upon helical wheel projection; and/or (ii) a segment of six adjacent residues comprising three to five K and/or R residues upon helical wheel projection, based on an alpha helix having angle of rotation between consecutive amino acids of 100 degrees and/or an alpha-helix having 3.6 residues per turn;
(e) said amphipathic alpha-helical motif comprises a hydrophobic outer face comprising: (i) at least two adjacent L residues upon helical wheel projection; and/or (ii) a segment of ten adjacent residues comprising at least five hydrophobic residues selected from: L, I, F, V, W, and M, upon helical wheel projection, based on an alpha helix having angle of rotation between consecutive amino acids of 100 degrees and/or an alpha-helix having 3.6 residues per turn;
(f) said hydrophobic outer face comprises a highly hydrophobic core consisting of spatially adjacent L, I, F, V, W, and/or M amino acids representing from 12.5%, 13%, 13.5%, 14%, 14.5%, 15%, 15.5%, 16%, 16.5%, 17%, 17.5%, 18%, 18.5%, 19%, 19.5%, or 20%, to 25%, 30%, 35%, 40%, or 45% of the amino acids of the shuttle agent;
(g) the shuttle agent has a hydrophobic moment (μ) between a lower limit of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, and an upper limit of 9.5, 9.6, 9.7, 9.8, 9.9, 10.0, 10.1, 10.2, 10.3, or 10.5;
(h) the shuttle agent has a predicted net charge of between +3, +4, +5, +6, +7, +8, +9, to +10, +11, +12, +13, +14, or +15;
(i) the shuttle agent has a predicted pI of 10 to 13; or
(j) any combination of (a) to (i).

9. The composition of claim 7 or 8, wherein said shuttle agent respects at least one, at least two, at least three, at least four, at least five, at least six, at least seven, or all of the following parameters:
(8) the shuttle agent is composed of 36% to 64%, 37% to 63%, 38% to 62%, 39% to 61%, or 40% to 60% of any combination of the amino acids: A, C, G, I, L, M, F, P, W, Y, and V;
(9) the shuttle agent is composed of 1% to 29%, 2% to 28%, 3% to 27%, 4% to 26%, 5% to 25%, 6% to 24%, 7% to 23%, 8% to 22%, 9% to 21%, or 10% to 20% of any combination of the amino acids: N, Q, S, and T;
(10) the shuttle agent is composed of 36% to 80%, 37% to 75%, 38% to 70%, 39% to 65%, or 40% to 60% of any combination of the amino acids: A, L, K, or R;
(11) the shuttle agent is composed of 15% to 40%, 20% to 40%, 20 to 35%, or 20 to 30% of any combination of the amino acids: A and L;
(12) the shuttle agent is composed of 20% to 40%, 20 to 35%, or 20 to 30% of any combination of the amino acids: K and R;
(13) the shuttle agent is composed of 5 to 10% of any combination of the amino acids: D and E;
(14) the difference between the percentage of A and L residues in the shuttle agent (% A+L), and the percentage of K and R residues in the shuttle agent (K +R), is less than or equal to 9%, 8%, 7%, 6%, or 5%; and
(15) the shuttle agent is composed of 15 to 40%, 20% to 35%, or 20% to 30% of any combination of the amino acids: Q, Y, W, P, I, S, G, V, F, E, D, C, M, N, T, and H.

10. The composition of any one of claims 1 to 9, wherein said shuttle agent comprises a histidine-rich domain, optionally wherein the histidine-rich domain is:
(i) positioned towards the N terminus and/or towards the C terminus of the shuttle agent;
(ii) is a stretch of at least 3, at least 4, at least 5, or at least 6 amino acids comprising at least 50%, at least 55%, at least 60%, at least 65%, at least 70%, at least 75%, at least 80%, at least 85%, or at least 90% histidine residues; and/or comprises at least 2, at least 3, at least 4, at least 5, at least 6, at least 7, at least 8, or at least 9 consecutive histidine residues; or
(iii) both (i) and (ii).

11. The composition of any one of claims 1 to 10, wherein said shuttle agent comprises a flexible linker domain rich in serine and/or glycine residues (e.g., separating N-terminal and a C-terminal segments of the shuttle agent; or positioned N- and/or C-terminal of a central amphipathic cationic alpha helical domain).

12. The composition of any one of claims 1 to 11, wherein said shuttle agent comprises or consists of the amino acid sequence of:
(a) [X1]-[X2]-[linker]-[X3]-[X4] (Formula 1);
(b) [X1]-[X2]-[linker]-[X4]-[X3] (Formula 2);
(c) [X2]-[X1]-[linker]-[X3]-[X4] (Formula 3);
(d) [X2]-[X1]-[linker]-[X4]-[X3] (Formula 4);
(e) [X3]-[X4]-[linker]-[X1]-[X2] (Formula 5);
(f) [X3]-[X4]-[linker]-[X2]-[X1] (Formula 6);
(g) [X4]-[X3]-[linker]-[X1]-[X2] (Formula 7);
(h) [X4]-[X3]-[linker]-[X2]-[X1] (Formula 8);
(i) [linker]-[X1]-[X2]-[linker] (Formula 9);
(j) [linker]-[X2]-[X1]-[linker] (Formula 10);
(k) [X1]-[X2]-[linker] (Formula 11);
(l) [X2]-[X1]-[linker] (Formula 12);
(m) [linker]-[X1]-[X2] (Formula 13);
(n) [linker]-[X2]-[X1] (Formula 14);
(o) [X1]-[X2] (Formula 15); or
(p) [X2]-[X1] (Formula 16),
wherein:
[X1] is selected from: 2[Φ]-1[+]-2[Φ]-1[ζ]-1[+]-; 2[Φ]-1[+]-2[Φ]-2[+]-; 1[+]-1[Φ]-1[+]-2[Φ]-1[ζ]-1[+]-; and 1[+]-1[Φ]-1[+]-2[Φ]-2[+]-;
[X2] is selected from: -2[Φ]-1[+]-2[Φ]-2[ζ]-; -2[Φ]-1[+]-2[Φ]-2[+]-; -2[Φ]-1[+]-2[Φ]-1[+]-1[ζ]; -2[Φ]-1[+]-2[Φ]-1[ζ]-1[+]-; -2[Φ]-2[+]-1[Φ]-2[+]-; -2[Φ]-2[+]-1[Φ]-2[ζ]-; -2[Φ]-2[+]-1[Φ]-1[+]-1[ζ]-; and -2[Φ]-2[+]-1[Φ]-1[ζ]-1[+]-;
[X3] is selected from: -4[+]-A-; -3[+]-G-A-; -3[+]-A-A-; -2[+]-1[Φ]-1[+]-A-; -2[+]-1[Φ]-G-A-; -2[+]-1[Φ]-A-A-; or -2[+]-A-1[+]-A; -2[+]-A-G-A; -2[+]-A-A-A-; -1[Φ]-3[+]-A-; -1[Φ]-2[+]-G-A-; -1[Φ]-2[+]-A-A-; -1[Φ]-1[+]-1[Φ]-1[+]-A; -1[Φ]-1[+]-1[Φ]-G-A; -1[Φ]-1[+]-1[Φ]-A-A; -1[Φ]-1[+]-A-1[+]-A; -1[Φ]-1[+]-A-G-A; -1[Φ]-1[+]-A-A-A; -A-1[+]-A-1[+]-A; -A-1[+]-A-G-A; and -A-1[+]-A-A-A;
[X4] is selected from: -1[ζ]-2A-1[+]-A; -1[ζ]-2A-2[+]; -1[+]-2A-1[+]-A; -1[ζ]-2A-1[+]-1[ζ]-A-1[+]; -1[ζ]-A-1[ζ]-A-1[+]; -2[+]-A-2[+]; -2[+]-A-1[+]-A; -2[+]-A-1[+]-1[ζ]-A-1[+]; -2[+]-1[ζ]-A-1[+]; -1[+]-1[ζ]-A-1[+]-A; -1[+]-1[ζ]-A-2[+]; -1[+]-1[ζ]-A-1[+]-1[ζ]-A-1[+]; -1[+]-2[ζ]-A-1[+]; -1[+]-2[ζ]-2[+]; -1[+]-2[ζ]-1[+]-A; -1[+]-2[ζ]-1[+]-1[ζ]-A-1[+]; -1[+]-2[ζ]-1[ζ]-A-1[+]; -3[ζ]-2[+]; -3[ζ]-1[+]-A; -3[ζ]-1[+]-1[ζ]-A-1[+]; -1[ζ]-2A-1[+]-A; -1[ζ]-2A-2[+]; -1[ζ]-2A-1[+]-1[ζ]-A-1[+]; -2[+]-A-1[+]-A; -2[+]-1[ζ]-1[+]-A; -1[+]-1[ζ]-A-1[+]-A; -1[+]-2A-1[+]-1[ζ]-A-1[+]; and -1[ζ]-A-1[ζ]-A-1[+]; and
[linker] is selected from: -Gn-; -Sn-; -(GnSn)n-; -(GnSn)nGn-; -(GnSn)nSn-; -(GnSn)nGn(GnSn)n-; and -(GnSn)nSn(GnSn)n-;
wherein:
[4:1:0] is an amino acid which is: Leu, Phe, Trp, Ile, Met, Tyr, or Val, preferably Leu, Phe, Trp, or Ile;
[+] is an amino acid which is: Lys or Arg;
[ζ] is an amino acid which is: Gln, Asn, Thr, or Ser;
A is the amino acid Ala;
G is the amino acid Gly;
S is the amino acid Ser; and
n is an integer from 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 1 to 4,or 1 to 3.

13. The composition of any one of claims 1 to 12, wherein the shuttle agent comprises or consists of:
(i) the amino acid sequence any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364;
(ii) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 by no more than 1, 2, 3, 4, 5, 6, 7, 8, 9, or 10 amino acids (e.g., excluding any linker domains);
(iii) an amino acid sequence that is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 (e.g., calculated excluding any linker domains);
(iv) an amino acid sequence that differs from any one of SEQ ID NOs: 1 to 50, 58 to 78, 80 to 107, 109 to 139, 141 to 146, 149 to 161, 163 to 169, 171, 174 to 234, 236 to 240, 242 to 260, 262 to 285, 287 to 294, 296 to 300, 302 to 308, 310, 311, 313 to 324, 326 to 332, 338 to 342, 344, or 353 to 364 by only conservative amino acid substitutions (e.g., by no more than no more than 1, 2, 3, 4, 5, 6, 7, 8, 9, or 10 conservative amino acid substitutions, preferably excluding any linker domains), wherein each conservative amino acid substitution is selected from an amino acid within the same amino acid class, the amino acid class being: Aliphatic: G, A, V, L, and I; Hydroxyl or sulfur/selenium-containing: S, C, U, T, and M; Aromatic: F, Y, and W; Basic: H, K, and R; Acidic and their amides: D, E, N, and Q; or
(v) any combination of (i) to (iv).

14. The composition of any one of claims 1 to 13, wherein the shuttle agent comprises or consists of a variant of the synthetic peptide shuttle agent, the variant being identical to the synthetic peptide shuttle agent as defined in any one of claims 1 or 7 to 13, except having at least one amino acid being replaced with a corresponding synthetic amino acid having a side chain of similar physiochemical properties (e.g., structure, hydrophobicity, or charge) as the amino acid being replaced, wherein the variant increases cytosolic/nuclear delivery of said non-anionic polynucleotide analog cargo in eukaryotic cells as compared to in the absence of the synthetic peptide shuttle agent, preferably wherein the synthetic amino acid replacement:
(a) replaces a basic amino acids with any one of: α-aminoglycine, α,γ-diaminobutyric acid, ornithine, α,β-diaminopropionic acid, 2,6-diamino-4-hexynoic acid, β-(1-piperazinyl)-alanine, 4,5-dehydro-lysine, 6-hydroxylysine, ω,ω-dimethylarginine, homoarginine, ω,ω′-dimethylarginine, ω-methylarginine, β-(2-quinolyl)-alanine, 4-aminopiperidine-4-carboxylic acid, α-methylhistidine, 2,5-diiodohistidine, 1-methylhistidine, 3-methylhistidine, spinacine, 4-aminophenylalanine, 3-aminotyrosine, β-(2-pyridyl)-alanine, or β-(3-pyridyl)-alanine;
(b) replaces a non-polar (hydrophobic) amino acid with any one of: dehydro-alanine, β-fluoroalanine, β-chloroalanine, β-lodoalanine, α-aminobutyric acid, α-aminoisobutyric acid, azetidine-2-carboxylic acid, α-allylglycine, propargylglycine, tert-butylalanine, β-(2-thiazolyl)-alanine, thiaproline, 3,4-dehydroproline, tert-butylglycine, β-cyclopentylalanine, β-cyclohexylalanine, α-methylproline, norvaline, α-methylvaline, penicillamine, β,β-dicyclohexylalanine, 4-fluoroproline, 1-aminocyclopentanecarboxylic acid, pipecolic acid, 4,5-dehydroleucine, allo-isoleucine, norleucine, α-methylleucine, cyclohexylglycine, cis-octahydroindole-2-carboxylic acid, β-(2-thienyl)-alanine, phenylglycine, α-methylphenylalanine, homophenylalanine, 1,2,3,4-tetrahydroisoquinoline-3-carboxylic acid, β-(3-benzothienyl)-alanine, 4-nitrophenylalanine, 4-bromophenylalanine, 4-tert-butylphenylalanine, α-methyltryptophan, β-(2-naphthyl)-alanine, β-(1-naphthyl)-alanine, 4-iodophenylalanine, 3-fluorophenylalanine, 4-fluorophenylalanine, 4-methyltryptophan, 4-chlorophenylalanine, 3,4-dichloro-phenylalanine, 2,6-difluoro-phenylalanine, n-in-methyltryptophan, 1,2,3,4-tetrahydronorharman-3-carboxylic acid, β,β-diphenylalanine, 4-methylphenylalanine, 4-phenylphenylalanine, 2,3,4,5,6-pentafluoro-phenylalanine, or 4-benzoylphenylalanine;
(c) replaces a polar, uncharged amino acid with any one of: β-cyanoalanine, β-ureidoalanine, homocysteine, allo-threonine, pyroglutamic acid, 2-oxothiazolidine-4-carboxylic acid, citrulline, thiocitrulline, homocitrulline, hydroxyproline, 3,4-dihydroxyphenylalanine, β-(1,2,4-triazol-1-yl)-alanine, 2-mercaptohistidine, β-(3,4-dihydroxyphenyl)-serine, β-(2-thienyl)-serine, 4-azidophenylalanine, 4-cyanophenylalanine, 3-hydroxymethyltyrosine, 3-iodotyrosine, 3-nitrotyrosine, 3,5-dinitrotyrosine, 3,5-dibromotyrosine, 3,5-diiodotyrosine, 7-hydroxy-1,2,3,4-tetrahydroiso-quinoline-3-carboxylic acid, 5-hydroxytryptophan, thyronine, B-(7-methoxycoumarin-4-yl)-alanine, or 4-(7-hydroxy-4-coumarinyl)-aminobutyric acid; and/or
(d) replaces an acidic amino acid with any one of: γ-hydroxyglutamic acid, γ-methyleneglutamic acid, γ-carboxyglutamic acid, α-aminoadipic acid, 2-aminoheptanedioic acid, α-aminosuberic acid, 4-carboxyphenylalanine, cysteic acid, 4-phosphonophenylalanine, or 4-sulfomethylphenylalanine.

15. The composition of any one of claims 1 to 14, wherein the shuttle agent does not comprise a cell penetrating domain (CPD), a cell-penetrating peptide (CPP), or a protein transduction domain (PTD).

16. The composition of any one of claims 1 to 14, wherein the shuttle agent does not comprise a CPD fused to an endosome leakage domain (ELD).

17. The composition of any one of claims 1 to 14, wherein the shuttle agent comprises an endosome leakage domain (ELD) and/or a cell penetrating domain (CPD).

18. The composition of any one of claims 15 to 17, wherein:
(i) said ELD is or is from: an endosomolytic peptide; an antimicrobial peptide (AMP); a linear cationic alpha-helical antimicrobial peptide; a Cecropin-A/Melittin hybrid (CM) peptide; pH-dependent membrane active peptide (PAMP); a peptide amphiphile; a peptide derived from the N terminus of the HA2 subunit of influenza hemagglutinin (HA); CM18; Diphtheria toxin T domain (DT); GALA; PEA; INF-7; LAH4; HGP; HSWYG; HA2; EB1; VSVG; Pseudomonas toxin; melittin; KALA; JST-1; C(LLKK) 3 C; G(LLKK)₃G; or any combination thereof;
(ii) said CPD is or is from: a cell-penetrating peptide or the protein transduction domain from a cell-penetrating peptide; TAT; PTD4; Penetratin; pVEC; M918; Pep-1; Pep-2; Xentry; arginine stretch; transportan; SynB1; SynB3; or any combination thereof; or
(iii) both (i) and (ii).

19. The composition of any one of claims 1 to 18, wherein the shuttle agent is a cyclic peptide and/or comprises one or more D-amino acids.

20. The composition of any one of claims 1 to 19, wherein the shuttle agent increases the transduction efficiency and/or total amount of non-anionic polynucleotide analog cargo delivered intracellularly in the eukaryotic cells by at least 3, 3.5, 4, 4.5, 5, 5.5, 6, 6.5, 7, 7.5, 8, 8.5, 9, 9.5, or 10-fold over a corresponding negative control lacking said shuttle agent.

21. The composition of any one of claims 1 to 20, wherein the shuttle agent further comprises a chemical modification to one or more amino acids, wherein the chemical modification does not destroy the transduction activity of the synthetic peptide shuttle agent.

22. The composition of claim 21, wherein the chemical modification is at the N and/or C terminus of the shuttle agent.

23. The composition of claim 21 or 22, wherein the chemical modification is the addition of an acetyl group (e.g., an N-terminal acetyl group), a cysteamide group (e.g., a C-terminal cysteamide group), or a fatty acid (e.g., C4-C16 fatty acid, preferably N-terminal).

24. The composition of any one of claims 1 to 23, wherein the concentration of the non-anionic polynucleotide analog cargo and/or the synthetic peptide shuttle agent in the composition is at least 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μM.

25. The composition as defined in any one of claims 1 to 24:
(i) for use in modulating gene expression in the eukaryotic cells (e.g., gene expression knock-down, modified splicing (e.g., exon skipping), modification of miRNA activity and/or maturation, translational frameshift induction, RNA editing interference, or modification of poly-A tailing);
(ii) for use in therapy, wherein the non-anionic polynucleotide analog cargo modulates gene expression of a therapeutically relevant target RNA in the eukaryotic cells;
(iii) for use as an anti-viral, wherein the non-anionic polynucleotide analog cargo modulates gene expression of a viral gene required for virulence and/or pathogenicity;
(iv) for use in delivering a non-therapeutic non-anionic polynucleotide analog cargo as a diagnostic agent;
(v) for use in the manufacture of a medicament or diagnostic agent (e.g., formulated for topical, enteral/gastrointestinal (e.g., oral), or parenteral administration);
(vi) for use in treating cancer (e.g., skin cancer, basal cell carcinoma, nevoid basal cell carcinoma syndrome), inflammation or an inflammation-related disease (e.g., psoriasis, atopic dermatitis, ulcerative colitis, urticaria, dry eye disease, dry or wet age-related macular degeneration, digital ulcers, actinic keratosis, idiopathic pulmonary fibrosis), pain (e.g., chronic or acute), or a disease affecting the lungs (e.g., cystic fibrosis, asthma, chronic obstructive pulmonary disease (COPD), or idiopathic pulmonary fibrosis); or
(vii) any combination of (i) to (vi).

26. The composition of any one of claims 1 to 24, or the composition for use of claim 25, wherein the eukaryotic cells are animal cells, mammalian cells, human cells, stem cells, primary cells, immune cells, T cells, NK cells, dendritic cells, epithelial cells, skin cells, gastrointestinal cells, lung cells, or ocular cells.

27. A method for modifying gene expression in eukaryotic cells, the method comprising:
(a) providing a non-anionic polynucleotide analog cargo for intracellular delivery, the non-anionic polynucleotide analog cargo being designed to hybridize to an RNA of interest in the eukaryotic cells;
(b) providing a synthetic peptide shuttle agent that is independent from, or is not covalently linked to, said non-anionic polynucleotide analog cargo;
(c) contacting the eukaryotic cells with the non-anionic polynucleotide analog cargo in the presence of the synthetic peptide shuttle agent at a concentration sufficient to increase the transduction efficiency and/or cytosolic/nuclear delivery of the non-anionic polynucleotide analog cargo, as compared to in the absence of said synthetic peptide shuttle agent,
wherein the non-anionic polynucleotide analog cargo hybridizes to the RNA of interest upon cytosolic/nuclear delivery, thereby effecting gene expression modification.

28. The method of claim 27, which is an in vitro method (e.g., for a therapeutic and/or diagnostic purpose).

29. The method of claim 27, which is an in vivo method (e.g., for therapeutic and/or diagnostic purpose).

30. The method of any one of claims 27 to 29, wherein:
(i) the non-anionic polynucleotide analog cargo is as defined in any one of claims 1 to 6;
(ii) the synthetic peptide shuttle agent is as defined in any one of claims 1 or 7 to 23;
(iii) the eukaryotic cells are contacted a concentration of the non-anionic polynucleotide analog cargo and/or the synthetic peptide shuttle agent as defined in claim 24;
(iv) the method is for a use as defined in claim 25;
(v) the eukaryotic cells are as defined in claim 26; or
(vi) any combination of (i) to (v).

31. The composition of any one of claims 1 to 26, or the method of any one of claims 27 to 30, wherein the non-anionic polynucleotide analog cargo is a non-anionic antisense oligonucleotide targeting a gene of the Hedgehog pathway.

32. The composition of claim 31 or the method of claim 31, wherein the non-anionic antisense oligonucleotide targets Gli1 for knockdown.

33. The composition of claim 32 or the method of claim 32, wherein the wherein the non-anionic antisense oligonucleotide hybridizes to the polynucleotide sequence of any one of SEQ ID NOs: 365-368.

34. The composition of any one of claims 31 to 33, or the method of any one of claims 31 to 33, wherein the composition or method of for the treatment of Gorlin's syndrome and/or basal cell carcinoma.


SEQUENCE LISTING (368 sequences)

SEQ ID NO: 1 - protein, 35 aa, Artificial Sequence
  CM18-Penetratin-cys
KWKLFKKIGAVLKVLTTGRQIKIWFQNRRMKWKKC

SEQ ID NO: 2 - protein, 41 aa, Artificial Sequence
  TAT-KALA
YGRKKRRQRRRWEAKLAKALAKALAKHLAKALAKALKACEA

SEQ ID NO: 3 - protein, 35 aa, Artificial Sequence
  His-CM18-PTD4
HHHHHHKWKLFKKIGAVLKVLTTGYARAAARQARA

SEQ ID NO: 4 - protein, 43 aa, Artificial Sequence
  His-LAH4-PTD4
HHHHHHKKALLALALHHLAHLALHLALALKKAYARAAARQARA

SEQ ID NO: 5 - protein, 41 aa, Artificial Sequence
  PTD4-KALA
YARAAARQARAWEAKLAKALAKALAKHLAKALAKALKACEA

SEQ ID NO: 6 - protein, 34 aa, Artificial Sequence
  EB1-PTD4
LIRLWSHLIHIWFQNRRLKWKKKYARAAARQARA

SEQ ID NO: 7 - protein, 41 aa, Artificial Sequence
  His-CM18-PTD4-6Cys
HHHHHHKWKLFKKIGAVLKVLTTGYARAAARQARACCCCCC

SEQ ID NO: 8 - protein, 29 aa, Artificial Sequence
  CM18-PTD4
KWKLFKKIGAVLKVLTTGYARAAARQARA

SEQ ID NO: 9 - protein, 35 aa, Artificial Sequence
  CM18-PTD4-6His
KWKLFKKIGAVLKVLTTGYARAAARQARAHHHHHH

SEQ ID NO: 10 - protein, 41 aa, Artificial Sequence
  His-CM18-PTD4-His
HHHHHHKWKLFKKIGAVLKVLTTGYARAAARQARAHHHHHH

SEQ ID NO: 11 - protein, 30 aa, Artificial Sequence
  TAT-CM18
YGRKKRRQRRRCKWKLFKKIGAVLKVLTTG

SEQ ID NO: 12 - protein, 37 aa, Artificial Sequence
  FSD5
HHHHHHLLKLWSRLLKLWTQGRRLKAKRAKAHHHHHH

SEQ ID NO: 13 - protein, 34 aa, Artificial Sequence
  FSD10
KWKLARAFARAIKKLGGSGGGSYARALRRQARTG

SEQ ID NO: 14 - protein, 27 aa, Artificial Sequence
  FSD12
KWKLARAFARAIKKLYARALRRQARTG

SEQ ID NO: 15 - protein, 32 aa, Artificial Sequence
  FSD18
LLKLWSRLLKLWTQGGSGGGSGRRLKAKRAKA

SEQ ID NO: 16 - protein, 42 aa, Artificial Sequence
  FSD19
HHHHHHLLKLWSRLLKTWTQGRRLKAKSAQASTRQAHHHHHH

SEQ ID NO: 17 - protein, 31 aa, Artificial Sequence
  FSD21
HHHHHHFLKIWSRLIKIWTQGRRKGAQAAFR

SEQ ID NO: 18 - protein, 37 aa, Artificial Sequence
  FSD23
HHHHHHLLKLWSRLLKEWTQGRRLEAKRAEAHHHHHH

SEQ ID NO: 19 - protein, 31 aa, Artificial Sequence
  FSD120
HHHHHHFLKIWSRLIKIWTQGLRKGAQAAKR

SEQ ID NO: 20 - protein, 30 aa, Artificial Sequence
  FSD127
HHHHHHLLKIWSRLIKGWTQGWRTIAQALG

SEQ ID NO: 21 - protein, 31 aa, Artificial Sequence
  FSD129
FLKIWSRLIKIWTQGGSGGGSRRKGAQAAFR

SEQ ID NO: 22 - protein, 37 aa, Artificial Sequence
  FSD131
HHHHHHFLKIWSRLIKIWTQGRRKGAQAAFRHHHHHH

SEQ ID NO: 23 - protein, 30 aa, Artificial Sequence
  FSD134
LIRKWIHLIHSWFQNLRRLYARAAARQARA

SEQ ID NO: 24 - protein, 32 aa, Artificial Sequence
  FSD146
LLKLWSRLLKLWTQGGSPPPSGRRLKAKRAKA

SEQ ID NO: 25 - protein, 32 aa, Artificial Sequence
  FSD155
LLKLWSRLLKLWTQGGSGEGSGRRLKAKRAKA

SEQ ID NO: 26 - protein, 23 aa, Artificial Sequence
  FSD156
WIRLFTKLWRIFQQGKRIKAKRA

SEQ ID NO: 27 - protein, 29 aa, Artificial Sequence
  FSD157
WIRLFTKLWRIFQQGGSGGGSKRIKAKRA

SEQ ID NO: 28 - protein, 29 aa, Artificial Sequence
  FSD159
WIRLFTKLWRIFRQGGSGGGSKRIKAKAA

SEQ ID NO: 29 - protein, 25 aa, Artificial Sequence
  FSD162
ILKLWSRLIKIWTQGRRKKAQAAKR

SEQ ID NO: 30 - protein, 25 aa, Artificial Sequence
  FSD168
LLKIWSRLIKIWTQGRRLGARAQAR

SEQ ID NO: 31 - protein, 27 aa, Artificial Sequence
  FSD173
LLKIWSRLIKIWTQGRRLGARAARQAR

SEQ ID NO: 32 - protein, 33 aa, Artificial Sequence
  FSD174
LLKIWSRLIKIWTQGRRLGGSGGGSARAARQAR

SEQ ID NO: 33 - protein, 32 aa, Artificial Sequence
  FSD194
KWKLLKLWSRLLKLWGGSGGGSRRLKAKRAKA

SEQ ID NO: 34 - protein, 23 aa, Artificial Sequence
  FSD220
WARAFAKAWRIFQQGKRIKAKRA

SEQ ID NO: 35 - protein, 30 aa, Artificial Sequence
  FSD250
KWKLLKLWSRLLKLWGGSGGGSARAARQAR

SEQ ID NO: 36 - protein, 30 aa, Artificial Sequence; identical to SEQ ID NO: 35
  FSD250D
  MISC_FEATURE (1)..(30): All D-amino acids

SEQ ID NO: 37 - protein, 32 aa, Artificial Sequence
  FSD253
KWKLLKLWSRLLKLWGGSRGGRGSARAARQAR

SEQ ID NO: 38 - protein, 29 aa, Artificial Sequence
  FSD258
WKLLKLWSRLLKLWGGSGGGSARAARQAR

SEQ ID NO: 39 - protein, 30 aa, Artificial Sequence
  FSD262
KWKLLRLWSRLLRLWGGSGGGSARAARQAR

SEQ ID NO: 40 - protein, 31 aa, Artificial Sequence
  FSD263
KWKLLKLWSRLLKLWGGSGGGSARAAARQAR

SEQ ID NO: 41 - protein, 31 aa, Artificial Sequence
  FSD264
KWKLLKLWSRLLKLWGGSGGGSARAAARAAR

SEQ ID NO: 42 - protein, 32 aa, Artificial Sequence
  FSD265
KWKLLKLWSRLLKLWGGSGGGSQARAAARQAR

SEQ ID NO: 43 - protein, 32 aa, Artificial Sequence
  FSD268
KWKLLKLWSRLLKLWGGSGGGSQARAQARQAR

SEQ ID NO: 44 - protein, 33 aa, Artificial Sequence
  FSD286
KWKLLRALARLLKLAGGSGGGSQRRLGARAQAR

SEQ ID NO: 45 - protein, 31 aa, Artificial Sequence
  FSD271
KWKLLKLWSRLLKLWRGGSGGGSARAARQAR

SEQ ID NO: 46 - protein, 30 aa, Artificial Sequence
  FSD272
KWKLAKLWSRLLKLWGGSGGGSARAARQAR

SEQ ID NO: 47 - protein, 30 aa, Artificial Sequence
  FSD273
KWKLLRAWSRLLKLWGGSGGGSARAARQAR

SEQ ID NO: 48 - protein, 30 aa, Artificial Sequence
  FSD276
KWKLLKLWSRLLRAWGGSGGGSARAARQAR

SEQ ID NO: 49 - protein, 32 aa, Artificial Sequence; identical to SEQ ID NO: 43
  FSD268 Cyclic Amide
  MISC_FEATURE (1)..(32): Cyclic peptide covalent link between K1 and R32

SEQ ID NO: 50 - protein, 34 aa, Artificial Sequence
  FSD268 Disulfide
  MISC_FEATURE (1)..(32): Cyclic peptide disulfiude bond between C1 and C34
CKWKLLKLWSRLLKLWGGSGGGSQARAQARQARC

SEQ ID NO: 51 - protein, 34 aa, Artificial Sequence; identical to SEQ ID NO: 13
  FSD10 Scarmble

SEQ ID NO: 52 - protein, 32 aa, Artificial Sequence; identical to SEQ ID NO: 43
  FSD268 Scramble

SEQ ID NO: 53 - protein, 33 aa, Artificial Sequence
  FSD174 Scramble
LGRSGRIKIGGWSALASRARQARGLKIWTQGRL

SEQ ID NO: 54 - protein, 36 aa, Artificial Sequence
  FSN3
HHHHHHQFLCFWLNKMGKHNTVWHGRHLKCHKRGKG

SEQ ID NO: 55 - protein, 35 aa, Artificial Sequence
  FSN4
HHHHHHLLYLWRRLLKFWCAGRRVYAKCAKAYGCF

SEQ ID NO: 56 - protein, 31 aa, Artificial Sequence
  FSN7
LIKLWSRFIKFWTQGRRIKAKLARAGQSWFG

SEQ ID NO: 57 - protein, 19 aa, Artificial Sequence
  FSN8
HHHHHHFRKLWLAIVRAKK

SEQ ID NO: 58 - protein, 31 aa, Artificial Sequence
  FSD117
HHHHHHFLKFWSRLFKFWTQGRRKGAQAAFR

SEQ ID NO: 59 - protein, 31 aa, Artificial Sequence
  FSD118
HHHHHHILKIWSRLIKIWTQGRRKGAQAAIR

SEQ ID NO: 60 - protein, 32 aa, Artificial Sequence
  FSD119
HHHHHHFLKIWSRALIKIWTQGLRKGAQAAKR

SEQ ID NO: 61 - protein, 31 aa, Artificial Sequence
  FSD121
HHHHHHVLKIWSRLIKIWTQGRRKGAQAAVR

SEQ ID NO: 62 - protein, 31 aa, Artificial Sequence
  FSD122
HHHHHHFLKVWSRLVKVWTQGRRKGAQAAFR

SEQ ID NO: 63 - protein, 31 aa, Artificial Sequence
  FSD123
HHHHHHVLKVWSRLVKVWTQGRRKGAQAAVR

SEQ ID NO: 64 - protein, 31 aa, Artificial Sequence
  FSD124
HHHHHHFLKIWQRLIKIWQQGRRKGAQAAFR

SEQ ID NO: 65 - protein, 31 aa, Artificial Sequence
  FSD125
HHHHHHFLKIWNRLIKIWNNGRRKGANAAFR

SEQ ID NO: 66 - protein, 30 aa, Artificial Sequence
  FSD126
HHHHHHFLKIWSRLIKIWTQGWRTGAQAGF

SEQ ID NO: 67 - protein, 30 aa, Artificial Sequence; identical to SEQ ID NO: 20
  FSD127

SEQ ID NO: 68 - protein, 31 aa, Artificial Sequence
  FSD128
HHHHHHFLKIWSRLIKIWPQPRRKGAQAAFR

SEQ ID NO: 69 - protein, 31 aa, Artificial Sequence
  FSD130
LIKIWTQFLKIWSRGGSGGGSRRLGARAQAR

SEQ ID NO: 70 - protein, 31 aa, Artificial Sequence
  FSD132
HHHHHHRFAAQAGKRRGQTWIKILRSWIKLF

SEQ ID NO: 71 - protein, 31 aa, Artificial Sequence
  FSD133
HHHHFLHHSWIKKILRTWIRRGQQAGKFAAR

SEQ ID NO: 72 - protein, 29 aa, Artificial Sequence
  FSD135
LIRKWIHLIHSWFQNLRRLQARAQARQAR

SEQ ID NO: 73 - protein, 29 aa, Artificial Sequence
  FSD137
LLRKWSHLLHIWGGSGGGSQARAQARQAR

SEQ ID NO: 74 - protein, 33 aa, Artificial Sequence
  FSD138
KWKLARAFARAIKIFGGSGGGSQRRLKAKRAKA

SEQ ID NO: 75 - protein, 40 aa, Artificial Sequence
  FSD139
HHHHHHLIRLWSHLIHIWFQNRRLKWKKKYARAAARQARA

SEQ ID NO: 76 - protein, 46 aa, Artificial Sequence
  FSD140
HHHHHHLIRLWSHLIHIWFQNRRLKWKKKYARAAARQARAHHHHHH

SEQ ID NO: 77 - protein, 41 aa, Artificial Sequence
  FSD141
LIRLWSHLIHIWFQNRRLKWKKKGGSGGGSYARAAARQARA

SEQ ID NO: 78 - protein, 25 aa, Artificial Sequence
  FSD142
FLKIWSHLIHIWTQGRRLKAKRAKA

SEQ ID NO: 79 - protein, 22 aa, Artificial Sequence
  FSD143
LIRKWIHLIHSWFQGRRLGARA

SEQ ID NO: 80 - protein, 49 aa, Artificial Sequence
  FSD144
HHHHHHKKALLAHALHLLALLALHLAHALKKAYGRKKRRQRRRHHHHHH

SEQ ID NO: 81 - protein, 52 aa, Artificial Sequence
  FSD145
HHHHHHKKHLLAHALHLLALLALHLAHALAHLKKAYGRKKRRQRRRHHHHHH

SEQ ID NO: 82 - protein, 31 aa, Artificial Sequence
  FSD147
LLKLWTQLLKLWSRGGSGGGSRRLKAKRAKA

SEQ ID NO: 83 - protein, 28 aa, Artificial Sequence
  FSD148
HHHHHHMVTVLFRRLRIRRACGPPRVRV

SEQ ID NO: 84 - protein, 28 aa, Artificial Sequence
  FSD149
HHHHHHMVRVLTRFLRIGARCRRPPVVR

SEQ ID NO: 85 - protein, 30 aa, Artificial Sequence
  FSD150
HHHHHHWITWLFKRLKIRRAAGQSKFRIAG

SEQ ID NO: 86 - protein, 30 aa, Artificial Sequence
  FSD151
HHHHHHWITWLRKILKRFRKAAQSGFRIAG

SEQ ID NO: 87 - protein, 30 aa, Artificial Sequence
  FSD152
HHHHHHWITWLRKILKRFGKAAQSGFRIAR

SEQ ID NO: 88 - protein, 30 aa, Artificial Sequence
  FSD153
HHHHHHWITWLRKILKRLGGAAQSIITGGQ

SEQ ID NO: 89 - protein, 36 aa, Artificial Sequence
  FSD154
HHHHHHWITWLFKRLKIRRAAGGSGGGSQSKFRIAG

SEQ ID NO: 90 - protein, 23 aa, Artificial Sequence
  FSD158
WIRLFTKLWRIFRQGKRIKAKAA

SEQ ID NO: 91 - protein, 25 aa, Artificial Sequence
  FSD160
ILKLWSRLIKIWTQGRRLGAQAALR

SEQ ID NO: 92 - protein, 31 aa, Artificial Sequence
  FSD161
ILKLWSRLIKIWTQGGSGGGSRRLGAQAALR

SEQ ID NO: 93 - protein, 31 aa, Artificial Sequence
  FSD163
ILKLWSRLIKIWTQGGSGGGSRRKKAQAAKR

SEQ ID NO: 94 - protein, 25 aa, Artificial Sequence
  FSD164
LLKIWSRLIKIWTQGRRLGARAARA

SEQ ID NO: 95 - protein, 31 aa, Artificial Sequence
  FSD165
LLKIWSRLIKIWTQGGSGGGSRRKKARAARA

SEQ ID NO: 96 - protein, 26 aa, Artificial Sequence
  FSD166
LLKLWSRLIKIWTKGRRKKARAAQAR

SEQ ID NO: 97 - protein, 32 aa, Artificial Sequence
  FSD167
LLKLWSRLIKIWTKGGSGGGSRRKKARAAQAR

SEQ ID NO: 98 - protein, 31 aa, Artificial Sequence
  FSD169
LLKIWSRLIKIWTQGGSGGGSRRLGARAQAR

SEQ ID NO: 99 - protein, 25 aa, Artificial Sequence
  FSD170
LIKIWTQLLKIWSRGRRLGARAQAR

SEQ ID NO: 100 - protein, 25 aa, Artificial Sequence
  FSD171
  MISC_FEATURE (1)..(1): Acetyl
  MISC_FEATURE (25)..(25): Amide
  MISC_FEATURE (25)..(25): Cysteamide
LLKIWSRLIKIWTQGRRLGARAQAR

SEQ ID NO: 101 - protein, 31 aa, Artificial Sequence
  FSD172
LLKIWSRLIKIWTQGRRLGGSGGGSARAQAR

SEQ ID NO: 102 - protein, 29 aa, Artificial Sequence
  FSD175
LLKIWSRLIKIWTQGRRLGGSARAARQAR

SEQ ID NO: 103 - protein, 36 aa, Artificial Sequence
  FSD176
LLKIWSRLIKIWTQGRRLGGSGGGSGGSARAARQAR

SEQ ID NO: 104 - protein, 25 aa, Artificial Sequence
  FSD177
KLKIWSRLIRKWTKGLRLGAQAQAR

SEQ ID NO: 105 - protein, 31 aa, Artificial Sequence
  FSD178
KLKIWSRLIRKWTKGGSGGGSLRLGAQAQAR

SEQ ID NO: 106 - protein, 28 aa, Artificial Sequence
  FSD179
LLKIWSRLIKIWTQGRGRESRKPRKSRQ

SEQ ID NO: 107 - protein, 34 aa, Artificial Sequence
  FSD180
LLKIWSRLIKIWTQGGSGGGSRGRESRKPRKSRQ

SEQ ID NO: 108 - protein, 28 aa, Artificial Sequence
  FSD181
LLKIWSRLIKIWTQGLGLLVLRVRAGKR

SEQ ID NO: 109 - protein, 34 aa, Artificial Sequence
  FSD182
LLKIWSRLIKIWTQGGSGGGSLGLLVLRVRAGKR

SEQ ID NO: 110 - protein, 22 aa, Artificial Sequence
  FSD183
LLKIWSRLIKIWTQGRRLGARA

SEQ ID NO: 111 - protein, 24 aa, Artificial Sequence
  FSD184
LLKIWSRLIKIWTQGRRLGARAAR

SEQ ID NO: 112 - protein, 25 aa, Artificial Sequence
  FSD185
LLKIWSRLIKIWTQGRRLGARAARQ

SEQ ID NO: 113 - protein, 27 aa, Artificial Sequence
  FSD186
LLKIWSRLIKIWTQGRGLEARAPRKAR

SEQ ID NO: 114 - protein, 28 aa, Artificial Sequence
  FSD187
LLKIWSRLIKIWTQGRRLGARKPRKSRQ

SEQ ID NO: 115 - protein, 27 aa, Artificial Sequence
  FSD188
LLKIWSRLIKIWTQGRGRESRAARQAR

SEQ ID NO: 116 - protein, 27 aa, Artificial Sequence
  FSD189
LLKIWSRLIKIWTQGRRLGRAQRAQRA

SEQ ID NO: 117 - protein, 23 aa, Artificial Sequence
  FSD190
LLKIWSRLIKIWTQGRAQRAQRA

SEQ ID NO: 118 - protein, 32 aa, Artificial Sequence
  FSD191
HHHHHHLLKIWSRLIKIWTQGTRSKRAGLQFP

SEQ ID NO: 119 - protein, 31 aa, Artificial Sequence
  FSD192
HHHHHHLLKIWSRLIKIWTQGVGRVHRLLRK

SEQ ID NO: 120 - protein, 33 aa, Artificial Sequence
  FSD193
KWKLLKIWSRLIKIWRRLGGSGGGSARAARQAR

SEQ ID NO: 121 - protein, 25 aa, Artificial Sequence
  FSD195
LLKIWSRLIKIWTQGRRLKARAQAR

SEQ ID NO: 122 - protein, 25 aa, Artificial Sequence
  FSD196
LLKIWSRLIKIWTQGRRLGARAAAR

SEQ ID NO: 123 - protein, 25 aa, Artificial Sequence
  FSD197
LLKIWSRLIKIWTQGRRLKARAAAR

SEQ ID NO: 124 - protein, 27 aa, Artificial Sequence
  FSD198
WSRLIKIWTQGGSGGGSRRKGAQAAFR

SEQ ID NO: 125 - protein, 23 aa, Artificial Sequence
  FSD199
WSRLITKIWRIFTQGRRLGARAA

SEQ ID NO: 126 - protein, 23 aa, Artificial Sequence
  FSD200
WSRLITKIWRIFTQGRRLKARAA

SEQ ID NO: 127 - protein, 19 aa, Artificial Sequence
  FSD201
WSRLIKLWTQGRRLKARAA

SEQ ID NO: 128 - protein, 19 aa, Artificial Sequence
  FSD202
WIRLFKLWQQGKRIKAKRA

SEQ ID NO: 129 - protein, 21 aa, Artificial Sequence
  FSD203
WSRLIKIWTQGRRLGARAQAR

SEQ ID NO: 130 - protein, 21 aa, Artificial Sequence
  FSD204
LLKIWSRLIKIWTQGRRLGAR

SEQ ID NO: 131 - protein, 17 aa, Artificial Sequence
  FSD205
WSRLIKIWTQGRRLGAR

SEQ ID NO: 132 - protein, 21 aa, Artificial Sequence
  FSD206
KIWSRLIKIWTQGRRLGARAQ

SEQ ID NO: 133 - protein, 25 aa, Artificial Sequence
  FSD207
LAKAWARAIKIWTQGRRLGARAQAR

SEQ ID NO: 134 - protein, 32 aa, Artificial Sequence
  FSD208
KWKLARAFARAIKKLGGSGGGSRRLGARAQAR

SEQ ID NO: 135 - protein, 33 aa, Artificial Sequence
  FSD209
LLKIWSRLIKIWTQGGSGGGSYARALRRQARTG

SEQ ID NO: 136 - protein, 32 aa, Artificial Sequence
  FSD210
KWKLARAFARAIKKLGGSGGGSRRLKAKRAKA

SEQ ID NO: 137 - protein, 34 aa, Artificial Sequence
  FSD211
KWKLLKLWSRLLKLWGGSGGGSYARALRRQARTG

SEQ ID NO: 138 - protein, 25 aa, Artificial Sequence
  FSD212
WSRLLKLWGGSGGGSRRLKAKRAKA

SEQ ID NO: 139 - protein, 25 aa, Artificial Sequence
  FSD213
LLKLWSRLLKLWGGSGGGSRRLKAK

SEQ ID NO: 140 - protein, 21 aa, Artificial Sequence
  FSD214
WSRLLKLWGGSGGGSRRLKAK

SEQ ID NO: 141 - protein, 25 aa, Artificial Sequence
  FSD215
KLWSRLLKLWGGSGGGSRRLKAKRA

SEQ ID NO: 142 - protein, 27 aa, Artificial Sequence
  FSD216
LLKIWSRLIKIWTQGRGRSRKPRKSRQ

SEQ ID NO: 143 - protein, 22 aa, Artificial Sequence
  FSD217
KWKLKLWRLKGGSGGGSRRAKA

SEQ ID NO: 144 - protein, 31 aa, Artificial Sequence
  FSD218
KWKLKLWRLKSRLKLWRLKGGSGGGSRRAKA

SEQ ID NO: 145 - protein, 23 aa, Artificial Sequence
  FSD219
WIRLWTHLWHIWQQGKRIKAKRA

SEQ ID NO: 146 - protein, 24 aa, Artificial Sequence
  FSD221
WKLIRLFTRLIKIWGQRRLKAKRA

SEQ ID NO: 147 - protein, 22 aa, Artificial Sequence
  FSD222
LARAFARAIKIFGQRRLKAKRA

SEQ ID NO: 148 - protein, 28 aa, Artificial Sequence
  FSD223
LARAFARAIKIFQGGSGGGSRRLKAKRA

SEQ ID NO: 149 - protein, 25 aa, Artificial Sequence
  FSD224
KWKLARAFARAIKIFGQRRLKAKRA

SEQ ID NO: 150 - protein, 31 aa, Artificial Sequence
  FSD225
KWKLARAFARAIKIFGGSGGGSQRRLKAKRA

SEQ ID NO: 151 - protein, 27 aa, Artificial Sequence
  FSD226
KWKLARAFARAIKIFGQRRLGARAQAR

SEQ ID NO: 152 - protein, 33 aa, Artificial Sequence
  FSD227
KWKLARAFARAIKIFGGSGGGSQRRLGARAQAR

SEQ ID NO: 153 - protein, 27 aa, Artificial Sequence
  FSD228
KWKLARAFARAIKIFGQRRLKAKRAKA

SEQ ID NO: 154 - protein, 33 aa, Artificial Sequence; identical to SEQ ID NO: 74
  FSD229

SEQ ID NO: 155 - protein, 26 aa, Artificial Sequence
  FSD230
KWKLAKAWARALKLWGRRLGARAQAR

SEQ ID NO: 156 - protein, 33 aa, Artificial Sequence
  FSD231
KWKLARAFARAIKKLGGSGGGSRRLKAKRALKA

SEQ ID NO: 157 - protein, 32 aa, Artificial Sequence
  FSD232
KWKWARAWARAWKKWGGSGGGSRRLGARAQAR

SEQ ID NO: 158 - protein, 32 aa, Artificial Sequence
  FSD233
KLKLARALARALKKLGGSGGGSRRLGARAQAR

SEQ ID NO: 159 - protein, 32 aa, Artificial Sequence
  FSD234
KIKIARAIARAIKKIGGSGGGSRRLGARAQAR

SEQ ID NO: 160 - protein, 32 aa, Artificial Sequence
  FSD235
KFKFARAFARAFKKFGGSGGGSRRLGARAQAR

SEQ ID NO: 161 - protein, 39 aa, Artificial Sequence
  FSD236
KWKLLKLWSRLLKLWSRLLKLWGGSGGGSRRLGARAQAR

SEQ ID NO: 162 - protein, 39 aa, Artificial Sequence
  FSD237
KWKLLKLWTQLLKLWTQLLKLWGGSGGGSRRLGARAQAR

SEQ ID NO: 163 - protein, 32 aa, Artificial Sequence
  FSD238
KWKLLKIWSRLIKIWGGSGGGSQARAQARQAR

SEQ ID NO: 164 - protein, 31 aa, Artificial Sequence
  FSD239
KWKLLKIWTQLIKIWGGSGGGSQARQARQAR

SEQ ID NO: 165 - protein, 33 aa, Artificial Sequence
  FSD240
KWKALLALALHLAHLALHLKKAGRRKGAQAAFR

SEQ ID NO: 166 - protein, 33 aa, Artificial Sequence
  FSD241
KWKLARAFARAIKKLGGSGGGSYARAAARQARA

SEQ ID NO: 167 - protein, 36 aa, Artificial Sequence
  FSD243
LLKIWSRLIKIWTQGRRLGGSGGGSYARAAARQARA

SEQ ID NO: 168 - protein, 34 aa, Artificial Sequence
  FSD244
KWKLAKAWARALKLWGGSGGGSYARAAARKAKRA

SEQ ID NO: 169 - protein, 34 aa, Artificial Sequence
  FSD246
KWKLLKLWSRLLKLWGGSGGGSYARAAARKAKRA

SEQ ID NO: 170 - protein, 37 aa, Artificial Sequence
  FSD247
LLKIWSRLIKIWTQGRRLGGSGGGSYARAAARKAKRA

SEQ ID NO: 171 - protein, 30 aa, Artificial Sequence
  FSD248
KWKLAKAWARALKLWGGSGGGSARAARQAR

SEQ ID NO: 172 - protein, 30 aa, Artificial Sequence
  FSD250 Scramble
RGKLWSLSKLKGWGGARASKAQLARLGLWR

SEQ ID NO: 173 - protein, 30 aa, Artificial Sequence
  FSD250E
KWKLLELWSELLELWGGSGGGSARAARQAR

SEQ ID NO: 174 - protein, 30 aa, Artificial Sequence
  FSD251
KWKLLKLWSRLLKLWGGSGGGSAEAAEQAE

SEQ ID NO: 175 - protein, 32 aa, Artificial Sequence; identical to SEQ ID NO: 37
  FSD254

SEQ ID NO: 176 - protein, 32 aa, Artificial Sequence
  FSD255
KWKLLKLWGGSRLLKLWGGSGGGSARAARQAR

SEQ ID NO: 177 - protein, 33 aa, Artificial Sequence
  FSD256
KWKLLKLGRWSRLGLKLWGGSGGGSARAARQAR

SEQ ID NO: 178 - protein, 32 aa, Artificial Sequence
  FSD257
KWKLLKLWAASRLLKLWGGSGGGSARAARQAR

SEQ ID NO: 179 - protein, 33 aa, Artificial Sequence
  FSD259
KWKLLKLARWSRLALKLWGGSGGGSARAARQAR

SEQ ID NO: 180 - protein, 30 aa, Artificial Sequence
  FSD260
RWRLLRLWSRLLRLWGGSGGGSARAARQAR

SEQ ID NO: 181 - protein, 30 aa, Artificial Sequence
  FSD261
GGSLLKLWSRLLKLWGGSGGGSARAARQAR

SEQ ID NO: 182 - protein, 27 aa, Artificial Sequence
  FSD266
LLKLWSRLLKLWGGSGGGSARAARQAR

SEQ ID NO: 183 - protein, 31 aa, Artificial Sequence
  FSD267
KWKLLKLWSRLLKLWGGSGGGSYARAARYAR

SEQ ID NO: 184 - protein, 32 aa, Artificial Sequence
  FSD269
KWKLLKLWSRLLKLWGGSGGGSYARAYARYAR

SEQ ID NO: 185 - protein, 28 aa, Artificial Sequence
  FSD270
KWKLLKLWSRLLKLWGGSGGGSAAAAEK

SEQ ID NO: 186 - protein, 30 aa, Artificial Sequence
  FSD274
KWKLARAWSRLLKLWGGSGGGSARAARQAR

SEQ ID NO: 187 - protein, 30 aa, Artificial Sequence
  FSD275
KWKLLKLWSRLAKLWGGSGGGSARAARQAR

SEQ ID NO: 188 - protein, 30 aa, Artificial Sequence; identical to SEQ ID NO: 48
  FSD276

SEQ ID NO: 189 - protein, 30 aa, Artificial Sequence
  FSD277
KWKLLKLWSRLARAWGGSGGGSARAARQAR

SEQ ID NO: 190 - protein, 30 aa, Artificial Sequence
  FSD278
KWKLARAWSRLARAWGGSGGGSARAARQAR

SEQ ID NO: 191 - protein, 29 aa, Artificial Sequence
  FSD279
KWKLARALARAWSRGGSGGGSARAARQAR

SEQ ID NO: 192 - protein, 30 aa, Artificial Sequence
  FSD280
KWKLLKLWKRLLKKWGGSGGGSARAARQAR

SEQ ID NO: 193 - protein, 30 aa, Artificial Sequence
  FSD281
KWSLLKLWSALLKLWGGSGGGSARAARQAR

SEQ ID NO: 194 - protein, 30 aa, Artificial Sequence
  FSD282
KWKLWKLLSRLWKLLGGSGGGSARAARQAR

SEQ ID NO: 195 - protein, 30 aa, Artificial Sequence
  FSD283
KWKLARKFKRAIKKFGGSGGGSARAARQAR

SEQ ID NO: 196 - protein, 30 aa, Artificial Sequence
  FSD284
KWALARAFARAIAIFGGSGGGSARAARQAR

SEQ ID NO: 197 - protein, 30 aa, Artificial Sequence
  FSD285
LARAFARAIKIFGGSGGGSQRRLGARAQAR

SEQ ID NO: 198 - protein, 33 aa, Artificial Sequence
  FSD287
KWKLLRALARLLKALGGSGGGSQRRLGARAQAR

SEQ ID NO: 199 - protein, 32 aa, Artificial Sequence
  FSD288
KWKLLKWWSRLLKWWGGSGGGSQARAQARQAR

SEQ ID NO: 200 - protein, 32 aa, Artificial Sequence
  FSD289
KWKLLKFWSRLLKFWGGSGGGSQARAQARQAR

SEQ ID NO: 201 - protein, 32 aa, Artificial Sequence
  FSD290
KWKLLKLYSRLLKLYGGSGGGSQARAQARQAR

SEQ ID NO: 202 - protein, 32 aa, Artificial Sequence
  FSD291
KWKLLKLFSRLLKLFGGSGGGSQARAQARQAR

SEQ ID NO: 203 - protein, 32 aa, Artificial Sequence
  FSD292
KWKLLSLWSSLLSLWGGSGGGSQARAQARQAR

SEQ ID NO: 204 - protein, 32 aa, Artificial Sequence
  FSD293
KWKLLSLWSRLLSLWGGSGGGSQARAQARQAR

SEQ ID NO: 205 - protein, 32 aa, Artificial Sequence
  FSD2294
KWKLLKLWSSLLKLWGGSGGGSQARAQARQAR

SEQ ID NO: 206 - protein, 31 aa, Artificial Sequence
  FSD295
KWKLLKLWSLLKLWGGSGGGSQARAQARQAR

SEQ ID NO: 207 - protein, 34 aa, Artificial Sequence
  FSD296
KWKLLKLWSRLLKLWQQGGSGGGSQARAQARQAR

SEQ ID NO: 208 - protein, 34 aa, Artificial Sequence
  FSD297
KWKLLKLWSRLLKLWNNGGSGGGSQARAQARQAR

SEQ ID NO: 209 - protein, 32 aa, Artificial Sequence
  FSD298
SWSLLKLWSRLLKLWGGSGGGSQARAQARQAR

SEQ ID NO: 210 - protein, 30 aa, Artificial Sequence
  FSD299
KWKLLKLWSRLLKLWIKIFGQARAQARQAR

SEQ ID NO: 211 - protein, 30 aa, Artificial Sequence
  FSD300
KWKLLKLWSRLLKLWWRIFGQARAQARQAR

SEQ ID NO: 212 - protein, 29 aa, Artificial Sequence
  FSD301
GGSGGGSKWKLLKLWSRLLKLWGGSGGGS

SEQ ID NO: 213 - protein, 28 aa, Artificial Sequence
  FSD302
KWKLLKLWSRLLKLWGGGQARAQARQAR

SEQ ID NO: 214 - protein, 26 aa, Artificial Sequence
  FSD303
KWKLLKLWSRLLKLWGQARAQARQAR

SEQ ID NO: 215 - protein, 25 aa, Artificial Sequence
  FSD304
KWKLLKLWSRLLKLWQARAQARQAR

SEQ ID NO: 216 - protein, 32 aa, Artificial Sequence
  FSD305
KWKLLKLWSRLLKLWGGGGGGGQARAQARQAR

SEQ ID NO: 217 - protein, 28 aa, Artificial Sequence
  FSD306
KWKLLKLWSRLLKLWGGSGGGSQARQAR

SEQ ID NO: 218 - protein, 25 aa, Artificial Sequence
  FSD307
KWKLLKLWSRLLKLWGGSGGGSQAR

SEQ ID NO: 219 - protein, 32 aa, Artificial Sequence
  FSD308
KWKLLKLWSRLLKLWGGSGGGSGARAGARGAR

SEQ ID NO: 220 - protein, 32 aa, Artificial Sequence
  FSD309
KWKLLKLWSRLLKLWGGSGGGSQAGAQAGQAG

SEQ ID NO: 221 - protein, 32 aa, Artificial Sequence
  FSD310
KWKLLKLWSRLLKLWGGSGGGSQGRGQGRQGR

SEQ ID NO: 222 - protein, 30 aa, Artificial Sequence
  FSD311
KWKLLKLWSRLLKLWGGSGGGSRGGRGGGR

SEQ ID NO: 223 - protein, 28 aa, Artificial Sequence
  FSD312
WIRLFTKLWIFQQGGSGGGSKRIKAKRA

SEQ ID NO: 224 - protein, 29 aa, Artificial Sequence
  FSD313
WIRLFSRLWRIFQQGGSGGGSKRIKAKRA

SEQ ID NO: 225 - protein, 32 aa, Artificial Sequence
  FSD314
KWKWIRLFSRLWRIFQQGGSGGGSKRIKAKRA

SEQ ID NO: 226 - protein, 31 aa, Artificial Sequence
  FSD315
WIRLFSRLWRIFQQGGSGGGSQARAQARQAR

SEQ ID NO: 227 - protein, 34 aa, Artificial Sequence
  FSD316
KWKWIRLFSRLWRIFQQGGSGGGSQARAQARQAR

SEQ ID NO: 228 - protein, 30 aa, Artificial Sequence
  FSD317
WIRLFTKLWQIFQQGGGSGGGSARAARQAR

SEQ ID NO: 229 - protein, 30 aa, Artificial Sequence
  FSD318
WIRLFTKLWRIFQQGGGSGGGSARAARQAR

SEQ ID NO: 230 - protein, 28 aa, Artificial Sequence
  FSD319
KWKLLKLWSRLLKLWGGSGGGSAAAAQK

SEQ ID NO: 231 - protein, 28 aa, Artificial Sequence
  FSD320
KWKLLKLWSRLLKLWGGSGGGSAAAAQQ

SEQ ID NO: 232 - protein, 24 aa, Artificial Sequence
  FSD321
KWKLAKAWSRAIKIWGARAQARQA

SEQ ID NO: 233 - protein, 30 aa, Artificial Sequence
  FSD322
KWKLAKAWSRALKLWGGSGGGSARAQARQA

SEQ ID NO: 234 - protein, 22 aa, Artificial Sequence
  FSD323
WIRLFTRLIKIWGQRRLKAKRA

SEQ ID NO: 235 - protein, 22 aa, Artificial Sequence
  FSD324
WARAFARAWRIFQQRRLKAKRA

SEQ ID NO: 236 - protein, 25 aa, Artificial Sequence
  FSD325
WARAFARAWRIFQQRRLARAARQAR

SEQ ID NO: 237 - protein, 23 aa, Artificial Sequence
  FSD326
LARAFARAIKIFGQARAQARQAR

SEQ ID NO: 238 - protein, 23 aa, Artificial Sequence
  FSD327
LARAFARAIKIFGRRLKAKRAKA

SEQ ID NO: 239 - protein, 23 aa, Artificial Sequence
  FSD328
LARAFARAIKIFGRRLGARAQAR

SEQ ID NO: 240 - protein, 23 aa, Artificial Sequence
  FSD330
LARAFARALLKLWGQRRLKAKRA

SEQ ID NO: 241 - protein, 21 aa, Artificial Sequence
  FSD331
KWKLARAFARAGQRRLKAKRA

SEQ ID NO: 242 - protein, 22 aa, Artificial Sequence
  FSD332
KWKLARAFARAGRRLGARAQAR

SEQ ID NO: 243 - protein, 32 aa, Artificial Sequence
  FSD333
KWKLLRLLLRLLKKLGGSGGGSQARAQARQAR

SEQ ID NO: 244 - protein, 32 aa, Artificial Sequence
  FSD334
KWKLLRWLWRLLKKLGGSGGGSQARAQARQAR

SEQ ID NO: 245 - protein, 32 aa, Artificial Sequence
  FSD335
KWKLARLLLRALKKLGGSGGGSQARAQARQAR

SEQ ID NO: 246 - protein, 32 aa, Artificial Sequence
  FSD336
KWKLLRLFLRLFKKLGGSGGGSQARAQARQAR

SEQ ID NO: 247 - protein, 32 aa, Artificial Sequence
  FSD337
KWKLARWLWRALKKLGGSGGGSQARAQARQAR

SEQ ID NO: 248 - protein, 32 aa, Artificial Sequence
  FSD338
KWKLLRWFWRLFKKLGGSGGGSQARAQARQAR

SEQ ID NO: 249 - protein, 32 aa, Artificial Sequence
  FSD339
KWKLARLFLRAFKKLGGSGGGSQARAQARQAR

SEQ ID NO: 250 - protein, 32 aa, Artificial Sequence
  FSD340
KWKLARWFWRAFKKLGGSGGGSQARAQARQAR

SEQ ID NO: 251 - protein, 37 aa, Artificial Sequence
  FSD341
LLKIWSRLIKIWTQGRRLGGSGGGSYARALRRQARTG

SEQ ID NO: 252 - protein, 34 aa, Artificial Sequence
  FSD342
KWKLARWFWRAFKKLGGSGGGSYARALRRQARTG

SEQ ID NO: 253 - protein, 30 aa, Artificial Sequence
  FSD343
KWKLLQLWSRLLQLWGGSGGGSARAARQAR

SEQ ID NO: 254 - protein, 30 aa, Artificial Sequence
  FSD344
QWQLLKLWSRLLKLWGGSGGGSARAARQAR

SEQ ID NO: 255 - protein, 30 aa, Artificial Sequence
  FSD345
KLKLLKLWSRLLKLWGGSGGGSARAARQAR

SEQ ID NO: 256 - protein, 30 aa, Artificial Sequence
  FSD346
KFKLLKLFSRLLKLFGGSGGGSARAARQAR

SEQ ID NO: 257 - protein, 30 aa, Artificial Sequence
  FSD347
KWKLLKLLSRLLKLLGGSGGGSARAARQAR

SEQ ID NO: 258 - protein, 30 aa, Artificial Sequence
  FSD348
KWKLLKLLSRLLKLLGGGGGGGARAARQAR

SEQ ID NO: 259 - protein, 30 aa, Artificial Sequence
  FSD349
KWKWLKLWSRLWKLWGGSGGGSARAARQAR

SEQ ID NO: 260 - protein, 30 aa, Artificial Sequence
  FSD350
KWKLLKFWSRLLKFWGGSGGGSARAARQAR

SEQ ID NO: 261 - protein, 30 aa, Artificial Sequence
  FSD351
KWKLLKLFSRLFKLWGGSGGGSARAARQAR

SEQ ID NO: 262 - protein, 30 aa, Artificial Sequence
  FSD352
KWKLLKLWSRLIKIWGGSGGGSARAARQAR

SEQ ID NO: 263 - protein, 30 aa, Artificial Sequence
  FSD353
KWKLLKLQSRLLKLQGGSGGGSARAARQAR

SEQ ID NO: 264 - protein, 25 aa, Artificial Sequence
  FSD354
KWKLLKLWSRLLKLWGGSGGGSQGR

SEQ ID NO: 265 - protein, 25 aa, Artificial Sequence
  FSD355
KWKLLKLWSRLLKLWGGSGGGSGAR

SEQ ID NO: 266 - protein, 25 aa, Artificial Sequence
  FSD356
KWKLLKLWSRLLKLWGGSGGGSQAG

SEQ ID NO: 267 - protein, 25 aa, Artificial Sequence
  FSD357
KWKLLKLWSRLLKLWGGSGGGSRRR

SEQ ID NO: 268 - protein, 30 aa, Artificial Sequence
  FSD358
KWKLLHLWSRLLHLWGGSGGGSARAARQAR

SEQ ID NO: 269 - protein, 30 aa, Artificial Sequence
  FSD359
KWKLLKLWSKLLKLWGGGGGGGAKAAKQAK

SEQ ID NO: 270 - protein, 30 aa, Artificial Sequence
  FSD360
RWRLLRLWSRLLRLWGGGGGGGARAARQAR

SEQ ID NO: 271 - protein, 27 aa, Artificial Sequence
  FSD361
LLKLWSKLLKLWGGGGGGGAKAAKQAK

SEQ ID NO: 272 - protein, 27 aa, Artificial Sequence
  FSD362
LLRLWSRLLRLWGGGGGGGARAARQAR

SEQ ID NO: 273 - protein, 23 aa, Artificial Sequence
  FSD363
LLKLWSKLLKLWGGGAKAAKQAK

SEQ ID NO: 274 - protein, 23 aa, Artificial Sequence
  FSD364
LLRLWSRLLRLWGGGARAARQAR

SEQ ID NO: 275 - protein, 21 aa, Artificial Sequence
  FSD365
KWKLLKLWSRLLKLWGGGQAR

SEQ ID NO: 276 - protein, 18 aa, Artificial Sequence
  FSD366
KWKLWSRLLKLWGGGQAR

SEQ ID NO: 277 - protein, 19 aa, Artificial Sequence
  FSD367
KWKLLKLWSRLLKGGGQAR

SEQ ID NO: 278 - protein, 33 aa, Artificial Sequence
  FSD368
KWKLARAFARASRLLKLWGGSGGGSARAARQAR

SEQ ID NO: 279 - protein, 30 aa, Artificial Sequence
  FSD369
KWKLARAFARALKLWGGSGGGSARAARQAR

SEQ ID NO: 280 - protein, 30 aa, Artificial Sequence
  FSD370
KWKLLKLWSRLLKKLGGSGGGSARAARQAR

SEQ ID NO: 281 - protein, 32 aa, Artificial Sequence
  FSD371
KWKLLKLWSRLLKLWQQGGSGGGSARAARQAR

SEQ ID NO: 282 - protein, 36 aa, Artificial Sequence
  FSD372
KWKLARAFARAIKKLNNGGSGGGSYARALRRQARTG

SEQ ID NO: 283 - protein, 26 aa, Artificial Sequence
  FSD373
GGSGGGSLLKLWSRLLKLWGGSGGGS

SEQ ID NO: 284 - protein, 32 aa, Artificial Sequence
  FSD374
GGSGGGSLLKIWSRLIKIWTQGRRLGGSGGGS

SEQ ID NO: 285 - protein, 29 aa, Artificial Sequence
  FSD375
GGSGGGSKWKLARAFARAIKKLGGSGGGS

SEQ ID NO: 286 - protein, 26 aa, Artificial Sequence
  FSD376
GGSGGGSLARAFARAIKIFGGSGGGS

SEQ ID NO: 287 - protein, 21 aa, Artificial Sequence
  FSD377
GGGKWKLLKLWSRLLKLWGGG

SEQ ID NO: 288 - protein, 29 aa, Artificial Sequence
  FSD378
GGSGGGSKWKWIRLFSRWIRLFGGSGGGS

SEQ ID NO: 289 - protein, 30 aa, Artificial Sequence
  FSD379
KWKLSKLWSKLSKLWGGSGGGSARAARQAR

SEQ ID NO: 290 - protein, 32 aa, Artificial Sequence
  FSD381
LLKLAKALAKALKLLGGSGGGSQARAQARQAR

SEQ ID NO: 291 - protein, 26 aa, Artificial Sequence
  FSD382
LLKLAKALAKALKLLGQARAQARQAR

SEQ ID NO: 292 - protein, 29 aa, Artificial Sequence
  FSD383
LLKLLLKLLKLLGGSGGGSQARAQARQAR

SEQ ID NO: 293 - protein, 23 aa, Artificial Sequence
  FSD384
LAKALAKALKLLGQARAQARQAR

SEQ ID NO: 294 - protein, 29 aa, Artificial Sequence
  FSD385
LLKLLKLLLKLLGGSGGGSQARAQARQAR

SEQ ID NO: 295 - protein, 27 aa, Artificial Sequence
  FSD386
LLKLLKLLLKLLKLLGGGGKGGGKGGK

SEQ ID NO: 296 - protein, 18 aa, Artificial Sequence
  FSD387
QLQLLRLLLRLLKKLQLQ

SEQ ID NO: 297 - protein, 34 aa, Artificial Sequence
  FSD388
KWKLARAFSRAIKLLGGSGGGSYARALRRQARTG

SEQ ID NO: 298 - protein, 34 aa, Artificial Sequence
  FSD389
KWKLAKAFSKAIKLLGGSGGGSYAKALKKQAKTG

SEQ ID NO: 299 - protein, 17 aa, Artificial Sequence
  FSD390
KWKLWSKLLKLWSKLWK

SEQ ID NO: 300 - protein, 31 aa, Artificial Sequence
  FSD391
GGKGGKGGKWKLLKLWSRLLKLWGGKGGKGG

SEQ ID NO: 301 - protein, 31 aa, Artificial Sequence
  FSD392
GGWGGWGGKWKLLKLWSRLLKLWGGWGGWGG

SEQ ID NO: 302 - protein, 30 aa, Artificial Sequence
  FSD393
RAQRAARASGGGSGGWLKLLRSWLKLLKWK

SEQ ID NO: 303 - protein, 40 aa, Artificial Sequence
  FSD394
KWKLARAFARAIKIFGGSGGGSGGGKWKLARAFARAIKIF

SEQ ID NO: 304 - protein, 32 aa, Artificial Sequence
  FSD395
KLKLLKLLLKLLKKLGGSGGGSQAKAQAKQAK

SEQ ID NO: 305 - protein, 32 aa, Artificial Sequence
  FSD396
KLKLAKLLLKALKKLGGSGGGSQAKAQAKQAK

SEQ ID NO: 306 - protein, 32 aa, Artificial Sequence
  FSD397
KLKLAKALAKALKKLGGSGGGSQAKAQAKQAK

SEQ ID NO: 307 - protein, 32 aa, Artificial Sequence
  FSD398
KLKLLKALAKLLKKAGGSGGGSQAKAQAKQAK

SEQ ID NO: 308 - protein, 32 aa, Artificial Sequence
  FSD399
KLKLAKALLKALKKLGGSGGGSQAKAQAKQAK

SEQ ID NO: 309 - protein, 32 aa, Artificial Sequence
  FSD400
KLKAAKALAKALKALGGSGGGSQAKAQAKQAK

SEQ ID NO: 310 - protein, 37 aa, Artificial Sequence
  FSD401
GGSGGGSKWKLLKLWSRLLKLWGGSGGGSARAARQAR

SEQ ID NO: 311 - protein, 29 aa, Artificial Sequence
  FSD402
LLKLLLKLLKKLGGSGGGSQAKAQAKQAK

SEQ ID NO: 312 - protein, 29 aa, Artificial Sequence
  FSD403
LAKALAKALKKLGGSGGGSQAKAQAKQAK

SEQ ID NO: 313 - protein, 29 aa, Artificial Sequence
  FSD404
KLKLLLKLLKKLGGSGGGSQAKAQAKQAK

SEQ ID NO: 314 - protein, 30 aa, Artificial Sequence
  FSD406
KLKLLKLLLKLLKKLGGSGGGSAKAQAKQA

SEQ ID NO: 315 - protein, 30 aa, Artificial Sequence
  FSD407
KLKLLKLLLKLLKKLGGSGGGSAKAAKQAK

SEQ ID NO: 316 - protein, 25 aa, Artificial Sequence
  FSD408
KLKLLKLLLKLLKKLGGSGGGSQAG

SEQ ID NO: 317 - protein, 25 aa, Artificial Sequence
  FSD409
KLKLAKALAKALKKLGGSGGGSQAG

SEQ ID NO: 318 - protein, 32 aa, Artificial Sequence
  FSD410
KLKLLKLLLKLLKKLGGSGGGSLAKALAKLAK

SEQ ID NO: 319 - protein, 32 aa, Artificial Sequence
  FSD411
KLKLLKLLLKLLKKLGGSGGGSQAKALAKQAK

SEQ ID NO: 320 - protein, 25 aa, Artificial Sequence
  FSD412
KLKLLKLLLKLLKKLGGSGGGSLAG

SEQ ID NO: 321 - protein, 32 aa, Artificial Sequence
  FSD413
KLKLAKALAKALKKLGGSGGGSQAKALAKQAK

SEQ ID NO: 322 - protein, 36 aa, Artificial Sequence
  FSD414
LLKKLLHLLHSLLQNLKKLGGSGGGSQAKAQAKQAK

SEQ ID NO: 323 - protein, 36 aa, Artificial Sequence
  FSD415
LIRKWIHLIHSWFQNLRRLGGSGGGSQAKAQAKQAK

SEQ ID NO: 324 - protein, 29 aa, Artificial Sequence
  FSD416
GGSGGGSKWKLAKAWSRALKLWGGSGGGS

SEQ ID NO: 325 - protein, 26 aa, Artificial Sequence
  FSD417
GGSGGGSLAKAWSRALKLWGGSGGGS

SEQ ID NO: 326 - protein, 29 aa, Artificial Sequence
  FSD418
GGSGGGSKLKLLKLLLKLLKKLGGSGGGS

SEQ ID NO: 327 - protein, 29 aa, Artificial Sequence
  FSD419
GGSGGGSKLKLAKALAKALKKLGGSGGGS

SEQ ID NO: 328 - protein, 33 aa, Artificial Sequence
  FSD421
GGSGGGSLLKKLLHLLHSLLQNLKKLGGSGGGS

SEQ ID NO: 329 - protein, 27 aa, Artificial Sequence
  FSD422
HHHHHHKWKLARAFARAIKKLHHHHHH

SEQ ID NO: 330 - protein, 24 aa, Artificial Sequence
  FSD423
HHHHHHLARAFARAIKIFHHHHHH

SEQ ID NO: 331 - protein, 29 aa, Artificial Sequence
  FSD424
KWKLARAFARAIKKLGGSGGGSGGSGGGS

SEQ ID NO: 332 - protein, 32 aa, Artificial Sequence
  FSD425
KLKLAKALAKALKLLGGSGGGSQAKAQAKQAK

SEQ ID NO: 333 - protein, 33 aa, Artificial Sequence
  FSD426
KLKLAKALAKALKKLGGSGGGSKKLKAKKALKA

SEQ ID NO: 334 - protein, 30 aa, Artificial Sequence
  FSD427
LAKALAKALKKLGGSGGGSKKLKAKKALKA

SEQ ID NO: 335 - protein, 30 aa, Artificial Sequence
  FSD428
KLKLAKALAKALKKLGGSGGGSKKLKAKKA

SEQ ID NO: 336 - protein, 28 aa, Artificial Sequence
  FSD429
KLKLAKALAKALKKLGGSGGGSKKLKAK

SEQ ID NO: 337 - protein, 33 aa, Artificial Sequence
  FSD430
KLKLAKALAKALKLLGGSGGGSKKLKAKLALKA

SEQ ID NO: 338 - protein, 34 aa, Artificial Sequence
  FSD431
KWKLAKAFAKAIKKLGGSGGGSYAKALKKQAKTG

SEQ ID NO: 339 - protein, 32 aa, Artificial Sequence
  FSD432
KWKLARAFARAIKKLGGSGGGSQAKAQAKQAK

SEQ ID NO: 340 - protein, 34 aa, Artificial Sequence
  FSD433
KLKLAKALAKALKKLGGSGGGSYARALRRQARTG

SEQ ID NO: 341 - protein, 34 aa, Artificial Sequence
  FSD434
KWKLAKAFAKAIKKLGGSGGGSGGKGGKKQGKTG

SEQ ID NO: 342 - protein, 32 aa, Artificial Sequence
  FSD435
  MISC_FEATURE (1)..(32): Xaa is L-2,4-diaminobutyric acid
XLXLLXLLLXLLXXLGGSGGGSQAXAQAXQAX

SEQ ID NO: 343 - protein, 22 aa, Artificial Sequence
  FSD436
  MISC_FEATURE (1)..(22): Xaa is (2-naphthyl)-L-alanine
LARAXARAIKIXGQRRLKAKRA

SEQ ID NO: 344 - protein, 34 aa, Artificial Sequence; identical to SEQ ID NO: 13
  FSD438
  MISC_FEATURE (1)..(1): N-ter octanoic acid

SEQ ID NO: 345 - DNA, 25 bp, Artificial Sequence
  PMO-GFP
acagctcctcgcccttgctcaccat

SEQ ID NO: 346 - DNA, 25 bp, Artificial Sequence
  PMO-Gli1
gtcatcgagttgaacatggcgtctc

SEQ ID NO: 347 - DNA, 23 bp, Artificial Sequence
  PMO-Wnt1
gcaacagcgcccagagccccatg

SEQ ID NO: 348 - DNA, 25 bp, Artificial Sequence
  PMO-FITC
cctcttacctcagttacaatttata

SEQ ID NO: 349 - RNA, 25 nt, Artificial Sequence
  siRNA-GFP
acagcuccucgcccuugcucaccau

SEQ ID NO: 350 - RNA, 21 nt, Artificial Sequence
  siRNA-Gli1
aacuccacaggcauacaggau

SEQ ID NO: 351 - protein, 11 aa, Artificial Sequence
  PTD4
YARAAARQARA

SEQ ID NO: 352 - protein, 11 aa, Artificial Sequence
  TAT
YGRKKRRQRRR

SEQ ID NO: 353 - protein, 18 aa, Artificial Sequence
  CM18
KWKLFKKIGAVLKVLTTG

SEQ ID NO: 354 - protein, 30 aa, Artificial Sequence
  KALA
WEAKLAKALAKALAKHLAKALAKALKACEA

SEQ ID NO: 355 - protein, 16 aa, Artificial Sequence
  Penetratin
RQIKIWFQNRRMKWKK

SEQ ID NO: 356 - protein, 15 aa, Artificial Sequence
  FSD92
LLKIWSRLIKIWTQG

SEQ ID NO: 357 - protein, 19 aa, Artificial Sequence
  FSD418-19
KLKLLKLLLKLLLKLLKKL

SEQ ID NO: 358 - protein, 12 aa, Artificial Sequence
  FSD418-12-2
LLKLLLKLLKKL

SEQ ID NO: 359 - protein, 15 aa, Artificial Sequence
  FSD418-15
KLKLLKLLLKLLKKL

SEQ ID NO: 360 - protein, 32 aa, Artificial Sequence
  FSD440
GGSGGGSKWKLFKKIGAVLKVLTTGGGSGGGS

SEQ ID NO: 361 - protein, 40 aa, Artificial Sequence
  FSD441
GGSGGGSKKALLALALHHLAHLALHLALALKKAGGSGGGS

SEQ ID NO: 362 - protein, 44 aa, Artificial Sequence
  FSD443
GGSGGGSWEAKLAKALAKALAKHLAKALAKALKACEAGGSGGGS

SEQ ID NO: 363 - protein, 30 aa, Artificial Sequence
  FSD445
GWGLLKLWSRLLKLWGGSGGGSARAARQAR

SEQ ID NO: 364 - protein, 29 aa, Artificial Sequence
  FSD446
KWKLLKLWSRLLKLWGGSGGGSHTSDQTN

SEQ ID NO: 365 - DNA, 25 bp, Homo sapiens
  misc_feature (9)..(11): Start codon
gagacgccatgttcaactcgatgac

SEQ ID NO: 366 - DNA, 25 bp, Homo sapiens
aagtttgcgcttctcgcgggtggtc

SEQ ID NO: 367 - DNA, 25 bp, Homo sapiens
gaaatagaagggaggtgaggggcga

SEQ ID NO: 368 - DNA, 24 bp, Homo sapiens
catcctccagaacggcaagaggga